(19) United States
(12) Patent Application Publication
(10) Pub. No.: US 2026/0258132 A1
(43) Pub. Date: Sep. 3, 2026

(54) TREATMENT OF LUNG CANCER WITH ANTI-PD-1 AND CHEMOTHERAPY

(71) Applicant: BeOne Medicines I GmbH, Basel (CH)

(72) Inventors: Yiyuan MA, Beijing (CN); Tai QIN, Beijing (CN); Shiangjiin LEAW, Beijing (CN); Wenjuan ZHENG, Beijing (CN)

(73) Assignee: BeOne Medicines I GmbH, Basel (CH)

(21) Appl. No.: 19/540,437

(22) Filed: Feb. 13, 2026

Related U.S. Application Data
(63) Continuation of application No. PCT/CN2024/112302, filed on Aug. 15, 2024.

(30) Foreign Application Priority Data
Aug. 15, 2023 (WO) PCT/CN2023/113147

(51) Int. Cl. C07K 16/28; A61K 31/555; A61K 31/7048; A61K 33/243; A61K 39/00; A61K 47/26; A61P 35/00

(52) U.S. Cl. CPC C07K 16/2818; A61K 31/555; A61K 31/7048; A61K 33/243; A61K 47/26; A61P 35/00; A61K 2039/505; A61K 2039/54; A61K 2039/545; C07K 2317/52; C07K 2317/565

(57) ABSTRACT

The disclosure relates to treatment of lung cancer (in particular, small cell lung cancer) in a human subject with an anti-PD-1 antibody and, optionally, chemotherapy. In some embodiments, the small cell lung cancer is an advanced (e.g., locally advanced) or extensive stage (e.g., stage III or stage IV), unresectable or metastatic (e.g., with liver, lung or lymph node metastasis, e.g., with 2, 3 or more metastatic sites) lung cancer. In some embodiments, the chemotherapy is carboplatin and/or cisplatin, and optionally further etoposide. In some embodiments, the SCLC is small cell carcinoma. In some embodiments, the SCLC is combined small cell carcinoma. In some embodiments, the treatment is the first line treatment. In some embodiments, the claimed therapy is effective in the treatment of SCLC. In some embodiments, the human subject is a responder to treatment by at least one measure of response to treatment.

### CROSS-REFERENCE TO RELATED APPLICATIONS

This application is a bypass continuation of International Patent Application No. PCT/CN2024/112302, filed Aug. 15, 2024, which claims priority from International Patent Application No. PCT/CN2023/113147, filed Aug. 15, 2023. The contents of these applications are incorporated herein by reference in their entireties.

### SEQUENCE LISTING

The present application contains a sequence listing which has been submitted electronically in xml format, and is hereby incorporated by reference in its entirety. Said xml file was created on Feb. 13, 2026, is 127,198 bytes, and is named 138881_1558_Sequence_Listing.xml.

### FIELD

Disclosed herein is a method for the prevention, delay of progression or treatment of lung cancer (such as small cell lung cancer) in a subject in need thereof comprising administering an antibody that binds to anti-PD-1, or a fragment thereof, as described herein, optionally in combination with a chemotherapy.

### BACKGROUND

Lung cancer is the most common cancer worldwide with approximately 1.8 million new diagnoses and 1.59 million deaths worldwide in 2012, which corresponds to the highest incidence among cancers and most common cancer-related mortality (Ferlay et al., Int J Cancer. 2015; 136(5):E359-86.). Globally, across all cancer types, lung cancer is more common in men (16.8%) as compared to women (8.8%). About 57 in 100,000 people in the U.S. develop lung cancer.

Two main types of lung cancer are small cell lung cancer and non-small cell lung cancer. Non-small cell lung cancer (NSCLC) accounts for approximately 85% of all cases of lung cancer (Molina et al., Mayo Clin Proc 2008; 83:584-94.; Howlader et al., SEER Cancer Statistics Review, 1975-2012, National Cancer Institute. based on November 2014 SEER data submission, posted to the SEER web site, April 2015). Small cell lung cancer represents about 13%-15% of lung cancer diagnoses, and it is distinguished from NSCLC by its rapid growth time and early development of metastatic disease (Govindan et al. J Clin Oncol. 2006 Oct. 1; 24:4539-44). Nearly all cases of SCLC are attributable to cigarette smoking (Pesch et al. Int J Cancer 2012; 131:1210-9). Poor prognostic factors for survival in patients with SCLC include extensive-stage disease, poor Eastern Cooperative Oncology Group (ECOG) Performance Status, weight loss, and markers associated with excessive bulk of disease (e.g., lactate dehydrogenase) (Yip et al. Lung Cancer. 2000 June; 28:173-85.; Foster et al. Cancer. 2009 Jun. 15; 115:2721-31.).

Patients with limited-stage SCLC (LS-SCLC) can be treated with chemotherapy and radiation with the potential for long-term survival (Stinchcombe et al. Oncologist 2010 February; 15:187-95.). However, the treatment cures only approximately 20% of patients with LS-SCLC, and those patients still suffer relapse within months of completing initial therapy (Simon et al. Chest. 2007 September; 132(3 Suppl):3245-3395.). The majority of patients with SCLC (about 70%) are not diagnosed until after the cancer has spread or they developed an extensive stage small cell lung cancer (Socinski et al., J Clin Oncol 2009; 27:4787-92.). There are fewer treatment options for extensive-stage disease (ES-SCLC) and it has poor survival prospects (median overall survival [OS] approximately 10 months) (Socinski et al. 2009). Chest pain, dyspnea, and cough are among the most frequent disease-related symptoms experienced by patients with lung cancer.

There is a long-standing unmet need in the art to effectively treat lung cancer, such as small cell lung cancer. In particular, there is a need to increase life span, survival rate and response to treatment in patients with lung cancer, such as extensive stage small cell lung cancer.

### SUMMARY

In some aspects, the present disclosure provides a method of treating of a lung cancer in a subject (e.g., a human) in need thereof, comprising administering to the human subject a therapeutically effective amount of an anti-PD-1 antibody or an antigen-binding fragment thereof, and a therapeutically effective amount of one or more chemotherapeutic drugs, wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36.

In some aspects, the present disclosure provides a method of treating of small cell lung cancer (SCLC) in a subject (e.g., a human) in need thereof, comprising administering to the human subject a therapeutically effective amount of an anti-PD-1 antibody or an antigen-binding fragment thereof, and a therapeutically effective amount of one or more chemotherapeutic drugs, wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36.

In some aspects, the present disclosure provides a method of treating of small cell lung cancer (SCLC) (e.g., extensive stage small cell lung cancer) in a human subject in need thereof, comprising administering to the human subject a therapeutically effective amount of an anti-PD-1 antibody or an antigen-binding fragment thereof, and a therapeutically effective amount of one or more chemotherapeutic drugs, wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; wherein the treating is clinically effective or the subject is a responder to the treating by at least one measure of response to treatment.

In some aspects, the disclosure provides a method of treating an extensive stage small cell lung cancer (ES-SCLC) in a human subject in need thereof, comprising administering to the human subject a therapeutically effective amount of an anti-PD-1 antibody or an antigen-binding fragment thereof, and a therapeutically effective amount of one or more chemotherapeutic drugs; wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; wherein the one or more chemotherapeutic drugs are (i) cisplatin or carboplatin, and (ii) etoposide; and wherein the treating is clinically effective or the subject is a responder to the treating by at least one measure of response to treatment.

In some aspects, the disclosure provides a method of treating a metastatic and/or stage IV small cell lung cancer in a human subject in need thereof, comprising administering to the human subject a therapeutically effective amount of an anti-PD-1 antibody or an antigen-binding fragment thereof, and a therapeutically effective amount of one or more chemotherapeutic drugs; wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; and wherein the one or more chemotherapeutic drugs are (i) cisplatin or carboplatin, and (ii) etoposide.

In some aspects, the disclosure provides a method of treating an extensive stage small cell lung cancer (ES-SCLC) in a human subject in need thereof, comprising administering to the human subject a therapeutically effective amount of an anti-PD-1 antibody or an antigen-binding fragment thereof, and a therapeutically effective amount of one or more chemotherapeutic drugs; wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; wherein the one or more chemotherapeutic drugs are (i) cisplatin or carboplatin, and (ii) etoposide; wherein the subject has a metastatic and/or stage IV ES-SCLC; and wherein the treating is clinically effective or the subject is a responder to the treating by at least one measure of response to treatment.

In some embodiments, the lung cancer is unresectable, locally advanced, and/or metastatic. In some embodiments, the lung cancer is unresectable (surgically unresectable). In some embodiments, the lung cancer is locally advanced. In some embodiments, the lung cancer is an extensive stage lung cancer. In some embodiments, the lung cancer is stage IIIA, stage IIIB or stage IV. In some embodiments, the lung cancer is stage IV lung cancer.

In some embodiments, the lung cancer is metastatic. In some embodiments, the subject has liver, lung, lymph node, bone and/or adrenal gland metastasis. In some embodiments, the subject has liver, lung and/or lymph node metastasis. In some embodiments, the subject has liver metastasis. In some embodiments, the subject has lung metastasis. In some embodiments, the subject has lymph node metastasis. In some embodiments, the subject has bone metastasis. In some embodiments, the subject has adrenal gland metastasis. In some embodiments, the subject has 1, 2, 3 or more metastatic sites. In some embodiments, the subject has 3 or more than 3 metastatic sites. In some embodiments, the subject does not have brain metastasis. In some embodiments, the subject has brain metastasis.

In some embodiments, the SCLC is small cell carcinoma. In some embodiments, the SCLC is combined small cell carcinoma.

In some embodiments, the subject has not been previously treated for the lung cancer or the treatment is a first line treatment. In some embodiments, the subject has not being previously treated with chemotherapy. In some embodiments, the subject has not being previously treated with immunotherapy.

In some embodiments, the lung cancer is chemotherapy-resistant. In some embodiments, the subject has progressed after prior chemotherapy. In some embodiments, the subject did not tolerate prior chemotherapy.

In some embodiments, the heavy chain variable region of the anti-PD-1 antibody or fragment comprises the amino acid sequence of SEQ ID NO:24 and/or the light chain variable region of the anti-PD-1 antibody comprises the amino acid sequence of SEQ ID NO:26.

In some embodiments, the anti-PD-1 antibody comprises an IgG constant region comprising any one of the amino acid sequences: SEQ ID NO:83, SEQ ID NO:84, SEQ ID NO:85, SEQ ID NO:86, SEQ ID NO:87, SEQ ID NO:88, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105 and SEQ ID NO:106. In some embodiments, the anti-PD-1 antibody comprises an IgG constant region comprising an amino acid sequence of SEQ ID NO:88.

In some embodiments, the anti-PD-1 antibody or fragment is administered parenterally, for example intravenously (such as by IV infusion). In some embodiments, the anti-PD-1 antibody or fragment is administered intravenously in the amount of 200 mg. In some embodiments, the anti-PD-1 antibody or fragment is administered intravenously (e.g., by IV infusion) once every three weeks (e.g., on Day 1 of every three week cycle), for example in the amount of 200 mg. In some embodiments, the anti-PD-1 antibody or fragment is administered intravenously (e.g., by IV infusion) once every three weeks, for example in the amount of 2 mg/kg to 5 mg/kg.

In some embodiments, the one or more chemotherapeutic drug is a platinum chemotherapy drug. In some embodiments, the one or more chemotherapeutic drug is carboplatin or cisplatin (where the choice between carboplatin and cisplatin can be in physician's discretion). In some embodiments, the one or more chemotherapeutic drug comprises etoposide (e.g., in addition to carboplatin or cisplatin). In some embodiments, the cisplatin, carboplatin and/or etoposide is administered intravenously. In some embodiments, the cisplatin is administered intravenously at about 75 mg/m² or the carboplatin is administered intravenously at about area under the plasma or serum concentration-time curve 5 (AUC5). In some embodiments, the etoposide is administered intravenously at about 100 mg/m². In some embodiments, the administering of the chemotherapeutic drug (such as carboplatin, cisplatin and etoposide) is once every three weeks (Q3W). In some embodiments, carboplatin and cisplatin are to be administered once in every three week cycle (e.g., on Day 1). In some embodiments, etoposide is to be administered three times in every three week cycle (e.g., on Days 1, 2 and 3). In some embodiments, the intravenous administration is by IV infusion.

In some embodiments, the administering of the anti-PD-1 antibody or fragment, carboplatin or cisplatin, and etoposide, is for 4 cycles of three weeks each. In some embodiments, the anti-PD-1 antibody or fragment, carboplatin or cisplatin, and etoposide, are administered for 4 cycles of three weeks each (in an induction period), and then further the anti-PD-1 antibody or fragment is administered in a maintenance period for at least 1 three week cycle (for e.g., 1, 2, 3, 4, 5, 6, 7, 8, 9, 10, 11, 12, 13, 14, 15 or more cycles).

In some embodiments, the treating is clinically effective or the subject is a responder to the treating by at least one measure of response to treatment. In some embodiments, the response to treatment is measured by overall survival, progression-free or event-free survival, overall response or objective response rate, partial response, and/or duration of response. In some embodiments, the response to treatment is measured by tumor volume, size or diameter. In some embodiments, the treating reduces tumor volume, size or diameter by at least 5%, at least 10%, at least 15%, at least 20%, at least 25%, at least 30%, at least 35%, at least 40%, at least 45%, at least 50%, or at least 75%. In some embodiments, the treating reduces tumor volume, size or diameter by at least 55%, 60%, 65%, 70%, 80%, 85%, 90%, 95%, 98%, 995% or 100%.

In some embodiments, the subject has greater than 30% probability of survival at 24 months from the treatment initiation. In some embodiments, the treating is effective as demonstrated by patient population overall survival rate at 24 months from the treatment initiation of greater than 30%. In some embodiments, the subject has greater than 15% or 20% probability of event-free survival at 12 months from the treatment initiation. In some embodiments, the treating is effective as demonstrated by patient population event-free survival rate at 12 months from the treatment initiation of greater than 20%.

In some embodiments, the subject has been diagnosed with the lung cancer described herein. In some embodiments, the subject has been diagnosed using histological and/or cytological markers. In some embodiments, the subject has been diagnosed using a biopsy (e.g., solid biopsy or liquid biopsy).

In some embodiments, the SCLC is of neuroendocrine (NE)-high subtype. In some embodiments, the SCLC is of NE-low subtype. In some embodiments, the SCLC is characterized by expression of INSM1, CD56/NCAM, synptophysin and/or chromogranin A markers. In some embodiments, the lung cancer is characterized by more than 16.3 ng/ml of neuron-specific enolase (NSE), more than 66 pg/ml progastrin-releasing peptide (ProGRP), and/or more than 2.5 ng/ml or more than 10 ng/ml carcinoembryonic antigen (CEA).

In another aspect, the present disclosure relates to a method of treating lung cancer in a human subject in need thereof, comprising administering to the human subject: (a) a therapeutically effective amount of an anti-PD-1 antibody or an antigen-binding fragment thereof, wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; and (b) a therapeutically effective amount of a chemotherapeutic drug selected from the group consisting of a platinum compound, carboplatin, paclitaxel, nab-paclitaxel, and any combination thereof. In some embodiments, the anti-PD-1 antibody is Tislelizumab.

In some embodiments, the one or more chemotherapeutic drugs comprise pemetrexed and a platinum compound. In some embodiments, the platinum compound is carboplatin. In some embodiments, the one or more chemotherapeutic drugs comprise (a) carboplatin, and (b) paclitaxel or nab-paclitaxel.

In some embodiments, the lung cancer is unresectable, locally advanced, and/or metastatic.

In some embodiments, the anti-PD-1 antibody is formulated for intravenous injection and is intravenously administered.

In some embodiments, the anti-PD-1 antibody is administered at a dose from about 2 mg/kg to about 5 mg/kg, preferably the anti-PD-1 antibody is administered at a dose 200 mg tislelizumab every 3 weeks.

In some embodiments, the anti-PD-1 antibody is administered at a dose from about 50 to 800 mg, optionally wherein the anti-PD-1 antibody is administered at 100 mg, 150 mg, 200, mg, 250 mg, 300 mg, 350 mg, 400 mg, 450 mg, 500 mg, 550 mg, 600 mg, 650 mg, 700 mg, 750 mg, or 800 mg.

In some embodiments, the PD-1 antibody is administered every week, every 2 weeks, every 3 weeks, every 4 weeks, every 5 weeks, or every 6 weeks, preferably wherein the anti-PD-1 antibody is administered at 150 mg every two weeks, 300 mg every four weeks, or 400 mg every six weeks.

In some embodiments, the human subject has an elevated level of PD-L1 expression (protein and/or mRNA) by malignant cells and/or by infiltrating immune cells within a tumor of at least 1% compared to an appropriate control.

In some embodiments, the elevated level of PD-L1 expression is at least 1%, 2%, 5%, 10%, 20%, or 50%.

In some embodiments the patient is treated with a kinase inhibitor such as crizotinib, erlotinib, sunitinib, or anlotinib prior to, during, and/or after the treatment with an anti-PD-1 antibody (e.g. tislelizumab), preferably wherein the patient is treated with the kinase inhibitor after the treatment with an anti-PD-1 antibody (e.g. tislelizumab).

### BRIEF DESCRIPTION OF THE DRAWINGS

FIG. 1 is a schematic showing clinical trial treatment arms in the Induction and Maintenance treatments. Abbreviations: AUC, area under the plasma or serum concentration-time curve; ECOG PS, Eastern Cooperative Oncology Group performance status; IV, intravenously; PD, progressive disease; RECIST v1.1, Response Evaluation Criteria in Solid Tumors Version 1.1; vs, versus. a. Treatment may continue until the completion of the scheduled 4 cycles (only for Induction treatment period), disease progression per RECIST v1.1, loss of clinical benefit, unacceptable toxicity, or withdrawal of informed consent, whichever occurs first. Note: For Induction treatment, cycles in which no chemotherapy is given do not count toward the total number of chemotherapy cycles. b. Patients with radiographic progressive disease suspected to reflect pseudoprogression may continue treatment until progressive disease is confirmed by repeated imaging ≥4 weeks later (but not exceeding 6 to 8 weeks from the date of initial documentation of progressive disease); patients who could derive benefit from study drug after PD per RECIST v1.1 criteria, may continue treatment until any of the conditions as specified above occurs.

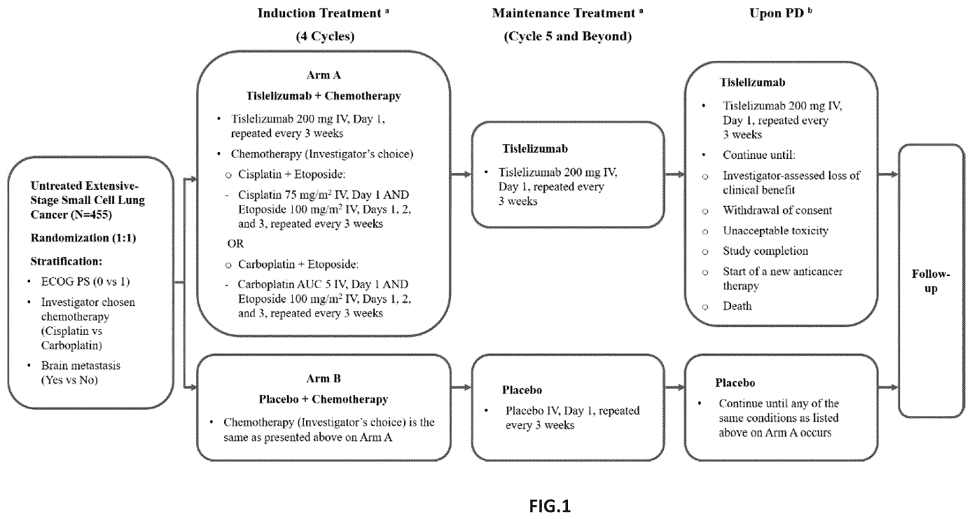

FIG. 2 provides a graph showing overall survival of subjects treated in Arm A (Arm A=Tislelizumab+Chemotherapy) and Arm B=Placebo+Chemotherapy, in the study described in Example 1.

FIG. 3 Provides a table showing rates of overall survival by subgroup of subjects treated in Arm A (Arm A=Tislelizumab+Chemotherapy) and Arm B=Placebo+Chemotherapy, in the study described in Example 1.

FIG. 4 provides a graph showing progression free survival of subjects treated in Arm A (Arm A=Tislelizumab+Chemotherapy) and Arm B=Placebo+Chemotherapy, in the study described in Example 1.

FIG. 5 provides a graph showing duration of response of subjects treated in Arm A (Arm A=Tislelizumab+Chemotherapy) and Arm B=Placebo+Chemotherapy, in the study described in Example 1.

FIG. 6 provides Kaplan-Meier plot of overall survival in patients treated with Tislelizumab+Chemotherapy) compared to treatment with Placebo+Chemotherapy, in the study described in Example 1.

FIG. 7 provides Kaplan-Meier plot of progression-free survival (ITT analysis set) of patients treated with Tislelizumab+Chemotherapy) compared to treatment with Placebo+Chemotherapy, in the study described in Example 1.

FIG. 8 provides Forest plot of progression-free survival by patient subgroups (ITT analysis set).

FIG. 9 provides a Kaplan-Meier plot of progression-free survival after next line of treatment (ITT analysis set) of patients treated with Tislelizumab+Chemotherapy) compared to treatment with Placebo+Chemotherapy, in the study described in Example 1.

FIG. 10 provides Kaplan-Meier plot of overall survival by cisplatin and carboplatin combination groups, in the study described in Example 1.

FIG. 11 provides Kaplan-Meier plot of progression-free survival by cisplatin and carboplatin combination groups, in the study described in Example 1.

### DETAILED DESCRIPTION

Provided herein are methods of treatment or delay or progression of lung cancer (e.g. small cell lung cancer) in a subject (e.g., human) by administering an anti-PD-1 antibody or an antigen-binding fragment thereof (e.g., tislelizumab or an anti-PD-1 antibody having CDRs of the tislelizumab) and, optionally, chemotherapy (carboplatin, cisplatin and/or etoposide). In some embodiments, the chemotherapy is carboplatin and/or cisplatin, and optionally further etoposide.

As detailed in the examples, it was surprisingly and unexpectedly discovered that the claimed methods result in effective treatment of lung cancer, which historically has been difficult to treat. In particular, the present application details the discovery of unexpected clinical benefits of the treatment regimen described herein for specified populations of human patients with lung cancer.

In some embodiments, provided herein are methods of treatment or delay or progression of advanced (e.g., locally advanced) extensive stage (e.g., stage III or stage IV), unresectable or metastatic (e.g., with liver, lung or lymph node metastasis, e.g., with 2, 3 or more metastatic sites) lung cancer (e.g. small cell lung cancer) in a subject (e.g., human) by administering an anti-PD-1 antibody or an antigen-binding fragment thereof (e.g., tislelizumab or an anti-PD-1 antibody having CDRs of the tislelizumab) and, optionally, chemotherapy (carboplatin, cisplatin and/or etoposide). In some embodiments, the chemotherapy is carboplatin and/or cisplatin, and optionally further etoposide.

In some embodiments, the small cell lung cancer is small cell carcinoma. In some embodiments, the small cell lung cancer is combined small cell carcinoma.

In some embodiments, the therapy is a first line therapy, i.e., the subject has not been treated (e.g., with chemotherapy and/or immunotherapy) for the lung cancer.

In some embodiments, the subject has progressed after prior chemotherapy or did not tolerate prior chemotherapy.

In some embodiments, the human subject is a responder to treatment by at least one measure of response to treatment. In some embodiments, the claimed therapy is effective in the treatment of the lung cancer (e.g., advanced, extensive stage, such as stage IV, or metastatic lung cancer). In some embodiments, the claimed therapy is effective in the treatment of the small cell lung cancer (e.g., advanced, extensive stage, such as stage IV, or metastatic lung cancer).

Experimental Data: Overall, tislelizumab in combination with chemotherapy as first-line treatment for patients with untreated ES-SCLC demonstrated significant clinical benefit and a manageable safety profile compared with placebo plus chemotherapy. In particular, as detailed in the examples below, the addition of tislelizumab to standard first-line chemotherapy with platinum and etoposide demonstrated a significant reduction in the risk of death for patients with ES-SCLC, which was further supported by improvements in PFS, ORR and DoR, and a manageable safety profile.

Surprising and unexpected aspects of the present invention include a demonstration that tislelizumab plus chemotherapy outperformed comparator combination therapies in similar studies. Further, an observed long-lasting treatment benefit throughout the study follow-up time was observed, as reflected by a higher 2-year survival probability estimated for tislelizumab plus chemotherapy over placebo plus chemotherapy. The OS benefit in the present study was sustained in the tislelizumab plus chemotherapy arm and was accompanied by improved PFS2 in favor of the tislelizumab arm, demonstrating the advantages of this therapy over chemotherapy in the first-line setting and regardless of subsequent anticancer treatment. A PFS benefit of tislelizumab plus chemotherapy versus placebo plus chemotherapy was consistently seen across patient subgroups.

The patient population enrolled in study detailed in the examples was broadly representative of patients with untreated ES-SCLC in China. SCLC primarily occurs in smokers, and the proportion of cases occurring in never-smokers has been reported to be only 2-5%. In the study detailed in the examples, the proportion of patients who were never-smokers (25%) was higher than global estimates (2.5%), consistent with the observed higher prevalence of never-smoking SCLC patients in Asia, as well as with prior phase 3 trials conducted in China in the same disease setting. The high incidence of never-smokers could be attributed to regional differences, while environmental (passive) tobacco smoking has been found to contribute to the development of SCLC in never-smokers.

The studies detailed in the examples, regarding tislelizumab plus chemotherapy, is the first phase 3 clinical trial demonstrating an OS benefit with an anti-PD-1 therapy combined with either cisplatin or carboplatin plus etoposide in this setting, providing new evidence that will broaden treatment options for patients with ES-SCLC.

### Definitions

For the present disclosure to be more readily understood, certain terms are first defined below. Additional definitions for the following terms and other terms are set forth throughout the Specification.

While various embodiments and aspects of the present disclosure are shown and described herein, it will be obvious to those skilled in the art that such embodiments and aspects are provided by way of example only. Numerous variations, changes, and substitutions will occur to those skilled in the art without departing from what is disclosed herein. It should be understood that various alternatives to the embodiments disclosed or described herein can be employed in practicing the instant disclosure.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, all technical and scientific terms used herein have the same meaning as commonly understood by one of ordinary skill in the art to which this disclosure is related. For example, Juo, The Concise Dictionary of Biomedicine and Molecular Biology, 2nd ed., (2001), CRC Press; The Dictionary of Cell & Molecular Biology, 5th ed., (2013), Academic Press; and “The Oxford Dictionary of Biochemistry and Molecular Biology,” Cammack et al. eds., 2nd ed, (2006), Oxford University Press, provide those of skill in the art with a general dictionary for many of the terms used in this disclosure. Additional definitions of common terms in molecular biology may be found in Benjamin Lewin, Genes V, published by Oxford University Press, 1994 (ISBN 0-19 854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8).

As used herein, including the appended claims, the singular forms of words such as “a,” “an,” and “the,” include their corresponding plural references unless the context clearly dictates otherwise. The term “or” is used to mean, and is used interchangeably with, the term “and/or” unless the context clearly dictates otherwise. The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

Ranges can be expressed herein as from “about” one particular value, and/or to “about” another particular value. When such a range is expressed, another case includes from the one particular value and/or to the other particular value. Similarly, when values are expressed as approximations, by use of the antecedent “about,” it will be understood that the particular value forms another case. It will be further understood that the endpoints of each of the ranges are significant both in relation to the other endpoint, and independently of the other endpoint. As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Systeme International de Unites (SI) accepted form. Numeric ranges are inclusive of the numbers defining the range.

The terms “at least”, “more than”, “or more”, and the like, e.g., “at least one” are understood to include but not be limited to at least 1, 2, 3, 4, 5, 6, 7, 8, 9, 10, 11, 12, 13, 14, 15, 16, 17, 18, 1920, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between. Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between. The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

The term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term. For example, in some embodiments, it will mean plus or minus 5% of the particular term. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “subject” or “patient” refers to any single subject for which therapy is desired or that is participating in a clinical trial, epidemiological study or used as a control, and includes any organism, preferably an animal, more preferably a mammal (e.g., rat, mouse, dog, cat, horse, cattle), and most preferably a human.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents, including cellular therapies.

Antibodies are large, complex molecules (molecular weight of ~150,000 or about 1320 amino acids) with intricate internal structure. The term “antibody” (Ab) includes, without limitation, a glycoprotein immunoglobulin which binds specifically to an antigen. In general, antibodies are large, complex molecules (molecular weight of ~150,000 or about 1320 amino acids) with an intricate internal structure. An antibody can comprise at least two heavy (H) chains and two light (L) chains interconnected by disulfide bonds, or an antigen-binding molecule thereof. Each H chain comprises a heavy chain variable region (abbreviated herein as HCVR or VH or Vh) and a heavy chain constant region. The heavy chain constant region comprises three constant domains, CH1, CH2 and CH3. Each light chain comprises a light chain variable region (abbreviated herein as LCVR or VL or Vl) and a light chain constant region. The term “antibody” as used herein refers to a polypeptide of the immunoglobulin family that can bind a corresponding antigen non-covalently, reversibly, and in a specific manner.

The light and heavy chain variable regions come together in 3-dimensional space to form a variable region that binds the antigen (for example, a receptor on the surface of a cell). The variable regions of each light/heavy chain (Vl/Vh) pair form the antibody binding site. In general, an intact antibody has two binding sites. Except in bifunctional or bispecific antibodies, the two binding sites are, generally, the same in primary sequence.

As will be presented in more detail below, within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The terms “CDR-L1,” “CDR-L2” and “CDR-L3” as provided herein refer to the complementarity determining regions (CDR) 1, 2, and 3 of the variable light (L) chain of an antibody. In embodiments, the variable light chain provided herein includes in N-terminal to C-terminal direction a CDR-L1, a CDR-L2 and a CDR-L3. Likewise, the terms “CDR-H1,” “CDR-H2” and “CDR-H3” as provided herein refer to the complementarity determining regions (CDR) 1, 2, and 3 of the variable heavy (H) chain of an antibody. In certain embodiments, the variable heavy chain provided herein includes in N-terminal to C-terminal direction a CDR-H1, a CDR-H2 and a CDR-H3. As detailed further below, the position and length of the CDRs have been precisely defined by Kabat, E. et al., Sequences of Proteins of Immunological Interest, U.S. Department of Health and Human Services, 1983, 1987. Naturally-produced antibodies are also glycosylated, e.g., on the CH2 domain.

The part of a variable region not contained in the CDRs is called the framework (“FR”), which forms the environment for the CDRs. Thus, the complementarity determining regions (CDRs) are interspersed with regions that are more conserved, i.e., the framework regions. Accordingly, each VH and VL comprises three CDRs and four FRs, arranged from amino-terminus to carboxy-terminus in the following order: FR1, CDR1, FR2, CDR2, FR3, CDR3, and FR4. While the variable regions of the heavy and light chains contain the binding domain that interacts with an antigen, the constant regions of the antibody may mediate the binding of the immunoglobulin to host tissues or factors, including various cells of the immune system (e.g., effector cells) and the first component (Clq) of the classical complement system.

TABLE 1
CDR Numbering:

| Loop | Kabat | AbM | Chothia | Contact |
|---|---|---|---|---|
| L1 | L24--L34 | L24--L34 | L24--L34 | L30--L36 |
| L2 | L50--L56 | L50--L56 | L50--L56 | L46--L55 |
| L3 | L89--L97 | L89--L97 | L89--L97 | L89--L96 |
| H1 | H31--H35B | H26--H35B | H26--H32 . . . 34 | H30--H35B |
| | (Kabat | | | |
| | Numbering) | | | |
| H1 | H31--H35 | H26--H35 | H26--H32 | H30--H35 |
| | (Chothia | | | |
| | Numbering) | | | |
| H2 | H50--H65 | H50--H58 | H52--H56 | H47--H58 |
| H3 | H95--H102 | H95--H102 | H95--H102 | H93--H101 |

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The positions of the CDRs and framework regions can be determined using various definitions well known in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M. P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996).

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia, the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT, the CDR amino acid residues in the VH are numbered approximately 26-35 (HCDR1), 51-57 (HCDR2) and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGapAlign.

In certain aspects, the CDRs of an antibody can be determined according to the Chothia numbering scheme, which refers to the location of immunoglobulin structural loops (see, e.g., Chothia C. & Lesk A. M., (1987), J Mol Biol 196: 901-917; Al-Lazikani B. et al., (1997) J Mol Biol 273: 927-948; Chothia C. et al., (1992) J Mol Biol 227: 799-817; Tramontano A. et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). In a specific embodiment, the CDRs of the antibodies described herein have been determined according to the Chothia numbering scheme.

When an antibody is said to comprise CDRs by a certain definition of CDRs (e.g., Kabat) that definition specifies the minimum number of CDR residues present in the antibody (i.e., the Kabat CDRs). It does not exclude the fact that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which HCDR1 is a composite Chothia-Kabat HCDR1 and other CDRs contain Kabat CDR residues and no additional CDR residues based on other definitions.

In some instances, a CDR is substantially identical to one found in a reference antibody (e.g., an antibody of the present disclosure) and/or the sequence of a CDR provided in the present disclosure. In some embodiments, a CDR is substantially identical to a reference CDR (e.g., a CDR provided in the present disclosure) in that it is either identical in sequence or contains between 1, 2, 3, 4, or 5 (e.g. 1-5) amino acid substitutions as compared with the reference CDR. In some embodiments a CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR (e.g., 85-90%, 85-95%, 85-100%, 90-95%, 90-100%, or 95-100%). In some embodiments a CDR is substantially identical to a reference CDR in that it shows at least 96%, 96%, 97%, 98%, 99%, or 100% sequence identity with the reference CDR. In some embodiments a CDR is substantially identical to a reference CDR in that one amino acid within the CDR is deleted, added, or substituted as compared with the reference CDR while the CDR has an amino acid sequence that is otherwise identical with that of the reference CDR. In some embodiments a CDR is substantially identical to a reference CDR in that 2, 3, 4, or 5 (e.g. 2-5) amino acids within the CDR are deleted, added, or substituted as compared with the reference CDR while the CDR has an amino acid sequence that is otherwise identical to the reference CDR. In various embodiments, an antigen binding fragment binds a same antigen as a reference antibody.

The term “antibody” further includes, but is not limited to, monoclonal antibodies, human antibodies, humanized antibodies, chimeric antibodies, and anti-idiotypic (anti-Id) antibodies. Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen binding fragments of any of the above. In certain embodiments, antibodies described herein refer to polyclonal antibody populations. Antibodies may also comprise, for example, Fab′ fragments, Fd′ fragments, Fd fragments, isolated CDRs, single chain Fvs, polypeptide-Fc fusions, single domain antibodies (e.g., shark single domain antibodies such as IgNAR or fragments thereof), camelid antibodies, single chain or Tandem diabodies (TandAb®), Anticalins®, Nanobodies® minibodies, BiTE®s, ankyrin repeat proteins or DARPINs®, Avimers®, DARTs, TCR-like antibodies, Adnectins®, Affilins®, Trans-Bodies®, Affibodies®, TrimerX®, MicroProteins, Fynomers®, Centyrins®, and KALBITOR®s.

An immunoglobulin may derive from any of the commonly known isotypes, including but not limited to IgA, secretory IgA, IgG, IgE, IgM, IgD, IgA and IgY IgG subclasses are also well known to those in the art and include but are not limited to human IgG1, IgG2, IgG3, IgG4, IgAQ1 and IgA2. The term “antibody” and “immunoglobulin” or “lg” may be used interchangeably herein.

The term “antibody” includes intact antibodies and binding fragments thereof. Typically, fragments compete with the intact antibody from which they were derived for specific binding to the target. Fragments include separate heavy chains, light chain Fab, Fab′, F(ab′)2, Fv fragments and single domain antibodies. Single (variable) domain antibodies include VH regions separated from their VL partners (or vice versa) in conventional antibodies (Ward et al., 1989, Nature 341: 544-546) as well as VH regions (sometimes known as VHH) from species such as Camelidae or cartilaginous fish (e.g., a nurse shark) in which VH regions are not associated with VL regions (see, e.g., WO 9404678). For example, natural single variable region antibodies from Camelidae include a VHH variable region, and CH2 and CH3 constant regions. Single domain antibodies can be subject to humanization by analogous approaches to conventional antibodies. The Dabs type of antibodies are usually obtained from antibodies of human origin. Nanobody types of antibodies are of Camelidae or shark origin and can be subject to humanization. Fragments can be produced by recombinant DNA techniques, or by enzymatic or chemical separation of intact immunoglobulins. The term “antibody” also includes bi-, tri- and multi-specific antibodies. For example, a bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

Antibodies can exist, e.g., as intact immunoglobulins or as a number of well-characterized fragments produced by digestion with various peptidases. For example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al. (1990)).

An “antigen binding molecule,” “antigen binding portion,” or “antibody fragment” refers to any molecule that comprises the antigen binding parts (e.g., CDRs) of the antibody from which the molecule is derived. Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In certain embodiments, the antigen binding molecule binds to PD-1. In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv).

Unless otherwise indicated, an “antigen-binding fragment” means antigen-binding fragments of antibodies, i.e., antibody fragments that retain the ability to bind specifically to the antigen bound by the full-length antibody, e.g., fragments that retain one or more CDR regions. Examples of antigen-binding fragments include, but not limited to, Fab, Fab′, F(ab′)2, and Fv fragments; diabodies; linear antibodies; single-chain antibody molecules, e.g., single chain Fv (ScFv); nanobodies and multi-specific antibodies formed from antibody fragments. In some embodiments, the antigen binding molecule binds to an antigen on a tumor cell. In certain embodiments, the antigen binding molecule binds to PD-1.

An antigen binding fragment may be produced by any means. For example, in some embodiments, an antigen binding fragment may be enzymatically or chemically produced by fragmentation of an intact antibody. In some embodiments, an antigen binding fragment may be recombinantly produced (i.e., by expression of an engineered nucleic acid sequence. In some embodiments, an antigen binding fragment may be wholly or partially synthetically produced. In some embodiments, an antigen binding fragment may have a length of at least about 50, 60, 70, 80, 90, 100, 110, 120, 130, 140, 150, 160, 170, 180, 190 amino acids or more; in some embodiments at least about 200 amino acids (e.g., 50-100, 50-150, 50-200, or 100-200 amino acids).

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor.

“Anti-tumor response” when referring to a cancer patient treated with a therapeutic regimen, such as a therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression-free survival. Positive therapeutic effects in cancer can be measured in a number of ways (See, e.g., Weber, W. A. “Assessing tumor response to therapy.” Journal of nuclear medicine 50.Suppl 1 (2009): 1S-10S); Eisenhauer et al., supra). In some embodiments, an anti-tumor response to a therapy described herein is assessed using RECIST 1.1 criteria, bidimensional irRC or unidimensional irRC. In some embodiments, an anti-tumor response is any of SD, PR, CR, PFS, or DFS.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Such conservatively modified variants are in addition to and do not exclude polymorphic variants, interspecies homologs, and alleles.

The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. The following eight groups each contain exemplary amino acids that may be considered conservative substitutions for one another (see, e.g., Creighton, Proteins (1984)).
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)

TABLE 2
Individual Exemplary Conservative Amino Acid Substitutions:

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions. In some embodiments, there may be 1, 2, 3, 4, 5, 6, 7, 8, 9, 10, 15, 20, 25, 30, 35, or 40 conservative substitutions. Thus, a “conservative amino acid substitution” refers to substitutions of amino acids in a protein with other amino acids having similar characteristics (e.g., charge, side-chain size, hydrophobicity/hydrophilicity, backbone conformation and rigidity, etc.) such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity.

The term “buffer” encompasses those agents which maintain the solution pH of the compositions of the present disclosure in an acceptable range.

“Chemotherapeutic agent” refers to a chemical or biological substance that can cause death of cancer cells, or interfere with growth, division, repair, and/or function of cancer cells. Classes of chemotherapeutic agents include, but are not limited to: microtubule inhibitors, e.g., taxanes (paclitaxel, docetaxel), and vinca alkaloids (vincristine, vinblastine, vinorelbine); antimetabolites (5-fluorouracil, capecitabine, gemcitabine), and antifolates (methotrexate, pemetrexed); kinase inhibitors (crizotinib, erlotinib, sunitinib, or anlotinib); topoisomerase inhibitors (deruxtecan, topotecan, irinotecan, anthracyclines, etoposide); cell-cycle independent drugs, e.g., platinum compounds and analogues (oxaliplatin), triazenes, alkylating agents (cyclophosphamide), spindle poison plant alkaloids (doxorubicin), cytotoxic/antitumor antibiotics (bleomycin, mithramycin, mitomycin), photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Current standard of care (SOC) treatments for certain cancers, such as colon, in the early-line setting include chemotherapy based on fluoropyrimidine, oxaliplatin, and irinotecan used in combination or sequentially, with option for monoclonal antibodies targeting vascular endothelial growth factor (VEGF) (e.g., bevacizumab, ziv-aflibercept) or its receptors (e.g., ramucirumab), and in patients with Ras wild type tumors, monoclonal antibodies targeting the epidermal growth factor (EGF) receptor (e.g., cetuximab, panitumumab). Treatment options for heavily pre-treated patients beyond the second-line setting are especially limited and associated toxicities can be severe.

“Co-administration” as used herein for agents such as the disclosed anti-PD-1 antibody, e.g., Tislelizumab, and a chemotherapeutic drug, means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the agents may be contained in separate vials, when in liquid solution, may be mixed into the same intravenous infusion bag or injection device, and administered simultaneously to the patient.

“Combination therapy” refers to those situations in which a subject is simultaneously exposed to two or more therapeutic regimens (e.g., two or more therapeutic moieties). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity). Such administration also encompasses co-administration in multiple, or in separate containers (e.g., capsules, powders, and liquids) for each active ingredient. Powders and/or liquids can be reconstituted or diluted to a desired dose prior to administration. Thus, the phrase “in combination with” means that an anti-PD-1 antibody is administered to a subject at the same time as, just before, or just after administration of an additional therapeutic agent. In certain embodiments, an anti-PD-1 antibody is administered as a co-formulation with an additional therapeutic agent. In some embodiments, the patient is treated with a chemotherapeutic agent after the treatment with an anti-PD-1 antibody. In some embodiments the patient is treated with a kinase inhibitor such as crizotinib, erlotinib, sunitinib, or anlotinib prior to, during, and/or after the treatment with an anti-PD-1 antibody (e.g. tislelizumab), preferably wherein the patient is treated with the kinase inhibitor after the treatment with an anti-PD-1 antibody (e.g. tislelizumab).

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

The terms “constant region” and “constant domain” are interchangeable and have a meaning common in the art. The constant region is an antibody portion, e.g., a carboxyl terminal portion of a light and/or heavy chain which is not directly involved in binding of an antibody to an antigen but which can exhibit various effector functions, such as interaction with the Fc receptor. The constant region of an immunoglobulin molecule generally has a more conserved amino acid sequence relative to an immunoglobulin variable domain.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.) such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth herein.

The term “dosage form” may be used to refer to a physically discrete unit of an active agent (e.g., an antigen binding system or antibody) for administration to a subject. Generally, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population. The total amount of a therapeutic composition or agent administered to a subject is determined by one or more medical practitioners and may involve administration of more than one dosage forms.

The term “dosing regimen” may be used to refer to a set of one or more unit doses that are administered individually to a subject. In some embodiments, a given therapeutic agent has a recommended dosing regimen, which may involve one or more doses. In some embodiments, a dosing regimen comprises a plurality of doses each of which is separated in time from other doses. In some embodiments, a dosing regimen comprises a plurality of doses and consecutive doses are separated from one another by time periods of equal length; in some embodiments, a dosing regimen comprises a plurality of doses and consecutive doses are separated from one another by time periods of at least two different lengths. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen is periodically adjusted to achieve a desired or beneficial outcome.

A “Durable Stable Disease Rate” means stable disease (SD) for ~23 weeks, where SD is cancer that is neither decreasing nor increasing in extent or severity.

“Effector cell” refers to a cell of the immune system that expresses one or more Fc receptors and mediates one or more effector functions. In some embodiments, effector cells may comprise, without limitation, one or more of monocytes, macrophages, neutrophils, dendritic cells, eosinophils, mast cells, platelets, large granular lymphocytes, Langerhans' cells, natural killer (NK) cells, T-lymphocytes, and B-lymphocytes. Effector cells may be of any organism comprising, without limitation, humans, mice, rats, rabbits, and monkeys.

“Effector function” refers to a biological result of interaction of an antibody Fc region with an Fc receptor or ligand. Effector functions comprise, without limitation, antibody-dependent cell-mediated cytotoxicity (ADCC), antibody-dependent cell-mediated phagocytosis (ADCP), and complement-mediated cytotoxicity (CMC). An effector function may be antigen binding dependent, antigen binding independent, or both. ADCC refers to lysis of antibody-bound target cells by immune effector cells. Without wishing to be bound by any theory, ADCC is generally understood to involve Fc receptor (FcR)-bearing effector cells recognizing and subsequently killing antibody-coated target cells (e.g., cells that express on their surface antigens to which an antibody is bound). Effector cells that mediate ADCC may comprise immune cells, comprising yet not limited to, one or more of natural killer (NK) cells, macrophages, neutrophils, eosinophils.

The term “excipient” refers to an agent that may be comprised in a composition, for example to provide or contribute to a desired consistency or stabilizing effect. In some embodiments, a suitable excipient may comprise, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

The term “heavy chain” when used in reference to an antibody can refer to any distinct type, e.g., alpha (α), delta (δ), epsilon (ε), γ (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, a “host cell” refers to any cell of any organism that is used for the purpose of producing a recombinant protein encoded by an expression vector or propagating the expression vector introduced into the host cell. A “mammalian recombinant host cell” refers to a mammalian host cell that comprises a heterologous expression vector, which may or may not be integrated into the host cell chromosome. A “bacterial recombinant host cell” refers to a bacterial host cell that comprises a heterologous expression vector, which may or may not be integrated into the host cell chromosome.

The term “human antibody” herein means an antibody that comprises human immunoglobulin protein sequences only. The variable and constant domain sequences generated may be assembled, or derived from human immunoglobulin sequences, or sequences indistinguishable therefrom. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

In some embodiments, antibodies (or antibody components) may be considered to be “human” even though their amino acid sequences comprise residues or elements not encoded by human germline immunoglobulin sequences (e.g., variations introduced by in vitro random or site-specific mutagenesis or introduced by in vivo somatic mutation).

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons. In some embodiments, a “humanized” antibody comprises one or more framework domains having substantially the amino acid sequence of a human framework domain, and one or more complementary determining regions having substantially the amino acid sequence as that of a non-human antibody. In some embodiments, a humanized antibody comprises at least a portion of an immunoglobulin constant region (Fc), generally that of a human immunoglobulin constant domain. In some embodiments, a humanized antibody may comprise a CH1, hinge, CH2, CH3, and, optionally, a CH4 region of a human heavy chain constant domain.

A “hybridoma” producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

The term “hypervariable region” means the amino acid residues of an antibody that are responsible for antigen-binding. The hypervariable region comprises amino acid residues from a “complementarity determining region” or “CDR” (i.e., CDR-L1, CDR-L2 and CDR-L3 in the light chain variable domain and CDR-H1, CDR-H2 and CDR-H3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” means those variable domain residues other than the hypervariable region residues defined herein as CDR residues.

Typically, the variable domains of both the heavy and light chains comprise three hypervariable regions, also called “complementarity determining regions (CDRs)”, which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5<m> ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al, (1989) Nature 342:878-883.

The term “identity” refers to the overall relatedness between polymeric molecules, e.g., between nucleic acid molecules (e.g., DNA molecules and/or RNA molecules) and/or between polypeptide molecules. The term “identical” or “percent identity” is a numeric score determined for a pair of aligned amino acid or nucleic acid sequences. Percent identity measures the number of identical residues (“identity”) between two sequences in relation to the length of the alignment across a “comparison window.” The number shows the % of amino acid residues or nucleotides that are the same between two sequences and indicates the degree of primary structure similarity.

Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally considering the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or 95-100%).

To calculate percent identity, the sequences being compared are typically aligned in a way that gives the largest match between the sequences. For sequence comparison, generally one sequence acts as a reference sequence, to which test sequences may be compared. When using a sequence comparison algorithm, test and reference sequences may be entered into a computer, subsequent coordinates may be designated, if necessary, and sequence algorithm program parameters may be designated. A polypeptide may be considered to be identical or substantially identical to a second polypeptide, for example, where the two peptides differ only by conservative substitutions. Methods of alignment of sequences for comparison are well-known in the art.

One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. Other algorithms are also available for comparison of amino acid or nucleic acid sequences, comprising those available in commercial computer programs such as BLASTN for nucleotide sequences and BLASTP, gapped BLAST, and PSI-BLAST for amino acid sequences. Exemplary such programs are described in Altschul et al., “Basic local alignment search tool,” J. Mol. Biol., 215(3): 403-410, 1990; Altschul et al., Methods in Enzymology; Altschul et al., “Gapped BLAST and PSI-BLAST: a new generation of protein database search programs,” Nucleic Acids Res. 25:3389-3402, 1997; Baxevanis et al., Bioinformatics: A Practical Guide to the Analysis of Genes and Proteins, Wiley, 1998; and Misener et al., (eds.), Bioinformatics Methods and Protocols (Methods in Molecular Biology, Vol. 132), Humana Press, 1999.

In addition to identifying similar sequences, the programs mentioned above generally provide an indication of the degree of similarity. In some embodiments, two sequences are considered to be substantially similar if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are similar and/or identical over a relevant stretch of residues (e.g., 85-90%, 85-95%, 85-100%, 90-95%, 90-100%, or 95-100%). In some embodiments, the relevant stretch is a complete sequence. In some embodiments, the relevant stretch is at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. Sequences with substantial sequence similarity may be homologs of one another.

Positive therapeutic effects in cancer can be measured in a number of ways (See, W. A Weber, J Nucl. Med. 50: I S-lOS (2009)). One common measure of efficacy is the T/C ratio, which is the ratio of tumor volume in control versus treated mice at a specified time, where T/C (%)=Median tumor volume of the treated/Median tumor volume of the control×100. With respect to tumor growth inhibition, according to NCI standards, a T/C<42% is the minimum level of anti-tumor activity. A T/C<10% is considered a high anti-tumor activity level. In some embodiments, the treatment achieved by administration of a composition of the present disclosure is any of progression free survival (PFS), disease free survival (DFS) or overall survival (OS). PFS, also referred to as “Time to Tumor Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the compositions, treatment methods, and uses of the present disclosure may not be effective in achieving a positive therapeutic effect in every patient, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, Id.), or other assays known in the art. Thus, under certain designated immunoassay conditions, the antibodies or antigen-binding fragments thereof specifically bind to a particular antigen at least two (2) fold when compared to the background level and do not specifically bind in a significant amount to other antigens present in the sample. In another aspect, under designated immunoassay conditions, the antibodies or antigen-binding fragments thereof, specifically bind to a particular antigen at least ten (10) fold when compared to the background level of binding and do not specifically bind in a significant amount to other antigens present in the sample.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies or binding fragments thereof, useful in the present invention will bind to the target protein with an affinity that is at least two fold greater, at least ten times greater, at least 20-times greater, or at least 100-times greater than the affinity with non-target proteins. An antibody herein is said to bind specifically to a polypeptide comprising a given amino acid sequence, e.g. the amino acid sequence of a mature human PD-1 molecule, if it binds to polypeptides comprising that sequence but does not bind to proteins lacking that sequence.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T and NK cell therapies. T and NK cell therapies can include adoptive cell therapies, tumor-infiltrating lymphocyte (TIL) immunotherapies, autologous cell therapies, engineered autologous cell therapy (eACT™), and allogeneic T and NK cell transplantation.

The terms “improve,” “increase,” “inhibit,” and “reduce” indicate values that are relative to a baseline or other reference measurement. In some embodiments, an appropriate reference measurement may comprise a measurement in certain system (e.g., in a single individual) under otherwise comparable conditions absent presence of (e.g., prior to and/or after) an agent or treatment, or in presence of an appropriate comparable reference agent. In some embodiments, an appropriate reference measurement may comprise a measurement in comparable system known or expected to respond in a comparable way, in presence of the relevant agent or treatment.

The term “isolated” refers to a substance that (1) has been separated from at least some components with which it was associated at an earlier time or with which the substance would otherwise be associated, and/or (2) is present in a composition that comprises a limited or defined amount or concentration of one or more known or unknown contaminants. An isolated substance, in some embodiments, may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% (e.g., 85-90%, 85-95%, 85-100%, 90-95%, 90-100%, or 95-100%) of other non-substance components with which the substance was associated at an earlier time, e.g., other components or contaminants with which the substance was previously or otherwise would be associated. In certain instances, a substance is isolated if it is present in a composition that comprises a limited or reduced amount or concentration of molecules of a same or similar type. For instance, in certain instances, a nucleic acid, DNA, or RNA substance is isolated if it is present in a composition that comprises a limited or reduced amount or concentration of non-substance nucleic acid, DNA, or RNA molecules. For instance, in certain instances, a polypeptide substance is isolated if it is present in a composition that comprises a limited or reduced amount or concentration of non-substance polypeptide molecules. In certain embodiments, an amount may be, e.g., an amount measured relative to the amount of a desired substance present in a composition. In certain embodiments, a limited amount may be an amount that is no more than 100% of the amount of substance in a composition, e.g., no more than 1%, 5%, 10%, 20%, 30%, 40%, 50%, 60%, 70%, 80%, 90%, or 95% of the amount of substance in a composition (e.g., 85-90%, 85-95%, 85-100%, 90-95%, 90-100%, or 95-100%). In certain instances, a composition is pure or substantially pure with respect to a selected substance. In some embodiments, an isolated substance is about 80%, about 85%, about 90%, about 91%, about 92%, about 93%, about 94%, about 95%, about 96%, about 97%, about 98%, about 99%, or more than about 99% pure (e.g., 85-90%, 85-95%, 85-100%, 90-95%, 90-100%, or 95-100%).

“Isotype” refers to the antibody class or subclass (e.g., IgM or IgG1) that is encoded by the heavy chain constant region genes. Thus, the term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman Ab may be humanized by recombinant methods to reduce its immunogenicity in man. Where not expressly stated, and unless the context indicates otherwise, the term “antibody” also includes an antigen binding fragment or an antigen-binding portion of any of the aforementioned immunoglobulins, and includes a monovalent and a divalent fragment or portion, and a single chain Ab.

The term “Kabat numbering” and like terms are recognized in the art and refer to a system of numbering amino acid residues in the heavy and light chain variable regions of an antibody, or an antigen-binding molecule thereof. In certain aspects, the CDRs of an antibody can be determined according to the Kabat numbering system (see, e.g., Kabat E. A. & Wu T. T. (1971) Ann NY Acad Sci 190: 382-391 and Kabat E. A. et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

The term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The term “monoclonal antibody” or “mAb” or “Mab” herein means a population of substantially homogeneous antibodies, i.e., the antibody molecules comprised in the population are identical in amino acid sequence except for possible naturally occurring mutations that can be present in minor amounts. In contrast, conventional (polyclonal) antibody preparations typically include a multitude of different antibodies comprising different amino acid sequences in their variable domains, particularly their complementarity determining regions (CDRs), which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art (see, e.g., Kohler et al., Nature 1975 256:495-497; U.S. Pat. No. 4,376,110; Ausubel et al., Current Protocols in Molecular Biology 1992; Harlow et al., Antibodies: A Laboratory Manual, Cold spring Harbor Laboratory 1988; and Colligan et al., Current Protocols in Immunology 1993).

“PD-L1 expression positive” refers to a Tumor Proportion Score, Mononuclear Inflammatory Density Score or Combined Positive Score of at least 1%; or elevated level of PD-L1 expression (protein and/or mRNA) by malignant cells and/or by infiltrating immune cells within a tumor compared to an appropriate control. In some embodiments, the elevated level of PD-L1 expression is at least 1%, 2%, 5%, 10%, 20%, or 50%.

The term “pharmaceutical composition” refers to preparations with pharmaceutically acceptable excipients which are in such form as to permit the active ingredients to be effective, and which contains no additional components which are toxic to the subjects to which the composition would be administered. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some aspects, the present disclosure provides compositions, e.g., pharmaceutically acceptable compositions, which include an anti-PD-1 antibody described herein, formulated together with at least one pharmaceutically acceptable excipient. In some aspects, the present disclosure provides compositions, e.g., pharmaceutically acceptable compositions, which include a chemotherapeutic drug described herein, formulated together with at least one pharmaceutically acceptable excipient.

The term “pharmaceutically acceptable” refers to a molecule or composition that, when administered to a recipient, is not deleterious to the recipient thereof, or that any deleterious effect is outweighed by a benefit to the recipient thereof.

Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

A “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection. In some embodiments, the antibody is administered by way of a syringe infusion system.

As used herein, “purifying” an antibody or antigen-binding fragment of interest or “purified composition” refers to increasing the degree of purity of the antibody or antigen binding fragment in the composition by removing (completely or partially) at least one impurity from the composition. The impurity can be host cell components such as serum, proteins or nucleic acids, cellular debris, growth medium or antibody aggregates. The term is not intended to refer to a complete absence of such biological molecules or to an absence of water, buffers, or salts or to components of a pharmaceutical composition that includes the antibody or antigen binding fragment.

“RECIST 1.1 Response Criteria” as used herein means the definitions set forth in Eisenhauer et al., Eur. J Cancer 45:228-247 (2009) for target lesions or nontarget lesions, as appropriate based on the context in which response is being measured.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

“Responder patient” when referring to a specific anti-tumor response to treatment with a therapy as described herein, means the patient exhibited the anti-tumor response.

The term “stage of cancer” refers to a qualitative or quantitative assessment of the level of advancement of a cancer. In some embodiments, criteria used to determine the stage of a cancer may comprise, without limitation, one or more of where the cancer is located in a body, tumor size, whether the cancer has spread to lymph nodes, whether the cancer has spread to one or more different parts of the body, etc. In some embodiments, cancer may be staged using the so-called TNM System, according to which T refers to the size and extent of the main tumor, usually called the primary tumor; N refers to the number of nearby lymph nodes that have cancer; and M refers to whether the cancer has metastasized. In some embodiments, a cancer may be referred to as Stage 0 (abnormal cells are present without having spread to nearby tissue, also called carcinoma in situ, or CIS; CIS is not cancer, though could become cancer), Stage I-III (cancer is present; the higher the number, the larger the tumor and the more it has spread into nearby tissues), or Stage IV (the cancer has spread to visceral organs and/or distant parts of the body). In some embodiments, a cancer may be assigned to a stage selected from the group consisting of: in situ; localized (cancer is limited to the place where it started, with no sign that it has spread); regional (cancer has spread to nearby lymph nodes, tissues, or organs): distant (cancer has spread to distant parts of the body); and unknown (there is not enough information to determine the stage).

The term “subject” in the context of the present disclosure is a mammal, e.g., a primate, preferably a higher primate, e.g., a human (e.g., a patient having, or at risk of having, a disorder described herein).

In general, two sequences are generally considered to be “substantially similar” if they contain a conservative amino acid substitution in corresponding positions. For example, certain amino acids are generally classified as “hydrophobic” or “hydrophilic” amino acids, and/or as having “polar” or “non-polar” side chains. Substitution of one amino acid for another of the same type may be considered a conservative substitution.

In general, an “effective amount” refers to an amount effective to treat a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated. More specifically, a “therapeutically effective amount,” “effective dose,” “effective amount,” or “therapeutically effective dosage” of a therapeutic agent, e.g., an antibody, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays. The “therapeutically effective amount” can vary with the antibody, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In the case of combination therapy, the “therapeutically effective amount” refers to the total amount of the combination objects for the effective treatment of a disease, a disorder or a condition.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and 30 lymphomas.

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC” refers to the set of criteria described in Nishino et al., Developing a Common Language for Tumor Response to Immunotherapy: Immune-related Response Criteria using Unidimensional measurements. Clin Cancer Res. 2013; 19(14):3936-3943. These criteria utilize the longest diameter (cm) of each lesion. The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Tumor Proportion Score (TPS)” refers to the percentage of tumor cells expressing PD-L1 or another tumor antigen, on the cell membrane at any intensity (weak, moderate or strong). Linear partial or complete cell membrane staining is interpreted as positive for PD-L1 or another antigen of interest.

The term “variable region” or “variable domain” as used herein means the segment of IgG chains which is variable in sequence between different antibodies. Typically, it extends to Kabat residue 109 in the light chain and 113 in the heavy chain. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VH”, “V”, LCVR” and “Vl domain” are used interchangeably to refer to the light chain variable region of an antibody.

The terms “VH”, “Vh”, “HCVR” and “Vh domain” are used interchangeably to refer to the heavy chain variable region of an antibody.

The variable regions of each light/heavy chain (Vl/Vh) pair form the antibody binding site. Thus, in general, an intact antibody has two binding sites. Except in bifunctional or bispecific antibodies, the two binding sites are, in general, the same.

In certain embodiments, a HCVR sequence having at least 82%, 83%, 84%, 85%, 86%, 87%, 88%, 89%, 90%, 91%, 92%, 93%, 94%, 95%, 96%, 97%, 98% or 99% amino acid sequence identity to the amino acid sequence of SEQ ID NO:24 contains substitutions (e.g., conservative substitutions), insertions, or deletions relative to the reference sequence, but an anti-PD-1 antibody or antigen-binding fragment thereof comprising that sequence retains the ability to specifically bind to PD-1. In certain embodiments, a total of 1 to 10 amino acids have been substituted, inserted and/or deleted in SEQ ID NO:24. In certain embodiments, a total of 1 to 5 amino acids have been substituted, inserted and/or deleted in SEQ ID NO:24. In certain embodiments, the substitutions, insertions, or deletions occur in regions outside the CDRs (i.e., in the FRs).

In certain embodiments, a LCVR sequence having at least 83%, 84%, 85%, 86%, 87%, 88%, 89%, 90%, 91%, 92%, 93%, 94%, 95%, 96%, 97%, 98% or 99% amino acid sequence identity to the amino acid sequence of SEQ ID NO:26 contains substitutions (e.g., conservative substitutions), insertions, or deletions relative to the reference sequence, but an anti-PD-1 antibody or antigen-binding fragment thereof comprising that sequence retains the ability to specifically bind to PD-1. In certain embodiments, a total of 1 to 10 amino acids have been substituted, inserted and/or deleted in SEQ ID NO:26. In certain embodiments, a total of 1 to 5 amino acids have been substituted, inserted and/or deleted in SEQ ID NO:26. In certain embodiments, the substitutions, insertions, or deletions occur in regions outside the CDRs (i.e., in the FRs).

### PD-1

Programmed Death-1 (PD-1, also known as Programmed Death-1, PD1, Pdcd-1 and CD279) is a 55 KD receptor protein related to CD28/CTLA4 co-stimulatory/inhibitory receptor family (Blank et al., 2005 Cancer Immunol Immunother 54:307-314). The genes and cDNAs coding for PD-1 were cloned and characterized in mouse and human (Ishida et al., 1992 EMBO J 11:3887-3395; Shinohara et al., 1994 Genomics 23:704-706). The full length PD-1 contains 288 amino acid residues (NCBI accession number: NP_005009). Its extracellular domain consists of amino acid residues 1-167, and the cytoplasmic C-terminal tail comprises residues 191-288, which has two hypothetical immune-regulatory motifs, an immunoreceptor tyrosine-based inhibitory motif (ITIM; Vivier et al., 1997 Immunol Today 18:286-291) and an immunoreceptor tyrosine switch motif (ITSM; Chemnitz et al., 2004 J Immunol 173:945-954).

To date, two sequence-related ligands, PD-L1 (B7-H1) and PD-L2 (B7-DC), have been identified to specifically interact with PD-1, inducing intracellular signal transduction that inhibits CD3 and CD28 mediated T-cell activation (Riley, 2009 Immunol Rev 229:114-125), which, in turn, attenuates T-cell activities, for example, reduction of cell proliferation, IL-2 and IFN-7 secretion, as well as other growth factor and cytokine secretion.

Expression of PD-1 was frequently found in immune cells such as T-cells, B-cells, monocytes and natural killer (NK) cells. It was rarely expressed in other human tissues, such as muscle, epithelium, neuronal tissues, etc. Furthermore, high level of PD-1 expression is often associated with activation of immune cells. For example, when human T-cell line, Jurkat, was activated by phytohaemagglutinin (PHA) or phorbol ester (12-O-tetradecanoylphorbol-13-acetate, or TPA), the expression of PD-1 was up-regulated visible in Western Blot (Vibharka et al., 1997 Exp Cell Res 232:25-28). The same phenomenon was observed in stimulated murine T- and B-lymphocytes and in primary human CD4⁺ T-cells upon stimulation by anti-CD3 antibody (Agata et al., 1996 Int Immunol 8:765-772; Bennett et al., 2003 J Immunol 170:711-118). The increase of PD-1 expression following stimulation of T effector cells redirects the activated T-effector cells towards exhaustion and reduced immune activities. Therefore, PD-1 mediated inhibitory signal plays an important role in immune tolerance (Bour-Jordan et al., 2011 Immunol Rev 241:180-205).

Increase of PD-1 expression in tumor-infiltrating lymphocytes (TILs) and PD-1 ligand expression in tumor cells were reported in varieties of cancers involved in different types of tissues and organs such as lung (Konishi et al., 2004 Clin Cancer Res 10:5094-5100), liver (Shi et al., 2008 Int J Cancer 128:887-896; Gao et al., 2009 Clin Cancer Res 15:971-979), stomach (Wu et al., 2006 Acta Histochem 108:19-24), kidney (Thompson et al., 2004 Proc Natl Acad Sci 101:17174-17179; Thompson et al., 2007 Clin Cancer Res 13:1757-1761), breast (Ghebeh et al., 2006 Neoplasia 8:190-198), ovary (Hamanishi et al. 2007 Proc Natl Acad Sci 104:3360-3365), pancreas (Nomi et al., 2007 Clin Cancer Res 13:2151-2157), melanocytes (Hino et al., 2010 Cancer 116:1757-1766) and esophagus (Ohigashi et al., 2005 Clin Cancer Res 11:2947-2953). More frequently, the increased expression of PD-1 and PD-L1 in those cancers is associated with poor prognosis of patient survival outcome. Transgenic mice with PD-1 gene knockout inhibiting xenograft cancer cell growth further elucidated the significance of PD-1 signaling in the modulation of immune system for cancer eradication or tolerance (Zhang et al., 2009 Blood 114:1545-1552).

PD-1 blockade can be accomplished by a variety of mechanisms including antibodies that bind PD-1 or its ligands. Examples of PD-1 and PD-L1 blockers, also named PD-1 and PD-L1 inhibitors, are described in U.S. Pat. Nos. 7,488,802; 7,943,743; 8,008,449; 8,168,757; 8,217,149, and WO03042402, WO2008156712, WO2010089411, WO2010036959, WO2011066342, WO2011159877, WO2011082400, WO2011161699, and WO2015035606, the entire contents of each of which are incorporated herein by reference.

### Anti-PD-1 Antibodies

In some embodiments, the methods described herein comprise administering an anti-PD-1 antibody or an antigen-binding fragment thereof. Any antibodies or antigen-binding fragments thereof that specifically bind to PD-1, particularly human PD-1, such as those described herein, can be used in compositions, combinations, uses and methods described herein.

In some embodiments, the anti-PD-1 antibody is any antibody known in the art or described herein. In some embodiments, the anti-PD-1 antibody is any antibody that specifically binds to PD-1. In some embodiments, the anti-PD-1 antibody inhibits PD-1-mediated cellular signaling and activity in immune cells. In some embodiments, the anti-PD-1 antibody binds to amino acid residues requires for its ligand binding and/or inhibits ligand binding.

In some embodiments, the antibodies and fragments described herein are isolated and purified.

In some embodiments, the present disclosure is directed to an antibody or antigen-binding fragment thereof, wherein the antibody is a monoclonal antibody, a chimeric antibody, a humanized antibody, a human engineered antibody, a single chain antibody (scFv), a Fab fragment, a Fab′ fragment, or a F(ab′)2 fragment.

In some embodiments, the anti-PD-1 antibody is a monoclonal antibody. In some embodiments, the anti-PD-1 antibody is a humanized antibody. In some embodiments, the anti-PD-1 antibody is a human antibody. In some embodiments, the anti-PD-1 antibody is a chimeric antibody.

In some embodiments, described herein are anti-PD-1 antibody, wherein the antibody is an immunoglobulin comprising any VH and VL regions described herein. The immunoglobulin molecules that can be used are of any type (e.g., IgG, IgE, IgM, IgD, IgY, IgA). The immunoglobulin molecules that can be used are of any class (e.g., IgG1, IgG2, IgG3, IgG4, IgA1, IgA2). The immunoglobulin molecules that can be used are of any subclass.

In some embodiments, the anti-PD-1 antibody is an IgG4 antibody. In some embodiments, the present disclosure is directed to an antibody or antigen-binding fragment thereof, wherein the Fc domain is of an IgG4.

In some embodiments, the anti-PD-1 antibody is an IgG1 antibody. In some embodiments, the present disclosure is directed to an antibody or antigen-binding fragment thereof, wherein the Fc domain is of an IgG1.

In some embodiments, the anti-PD-1 antibody is a monoclonal humanized IgG1 antibody.

In some embodiments, the anti-PD-1 antibody is a monoclonal humanized IgG4 antibody.

In some embodiments, the contemplated anti-PD-1 antibodies and fragments comprise any CDRs described herein. In some embodiments, the contemplated anti-PD-1 antibodies and fragments comprise CDRs of any of the antibodies described herein. Complementarity-determining regions (CDRs) are defined in various ways in the art, including the Kabat, Chothia, AbM, Contact, and IMGT. In some embodiments, the CDRs of an antibody are defined according to the Kabat system.

In some embodiments, the contemplated anti-PD-1 antibodies and fragments comprise any light chain variable region described herein and/or any heavy chain variable region described herein. In some embodiments, the described anti-PD-1 antibodies and fragments comprise a light chain variable region having a sequence with at least 85%, 90% or 95% identity to any light chain variable region described herein and/or a heavy chain variable region having a sequence with at least 85%, 90% or 95% identity to any heavy chain variable region described herein. In some embodiments, the described anti-PD-1 antibodies and fragments comprise a light chain variable region having a sequence with at least 85%, 90% or 95% identity to any light chain variable region described herein (with at least 90%, 95%, 97%, 99% or 100% identity in the CDR regions, e.g., the six CDRs) and/or a heavy chain variable region having a sequence with at least 85%, 90% or 95% identity to any heavy chain variable region described herein (with at least 90%, 95%, 97%, 99% or 100% identity in the CDR regions, e.g., the six CDRs). In some embodiments, the contemplated anti-PD-1 antibodies and fragments comprise light chain variable region and/or any heavy chain variable region of any antibody described herein.

In some embodiments, the anti-PD-1 antibody is any antibody described in WO2015/035606 A1 or US2015-0315274, the entire disclosures of which are incorporated by reference herein in their entireties, and in particular in respect to description therein of anti-PD-1 antibodies. In some embodiments, the anti-PD-1 antibody is any antibody described in any of: U.S. Pat. Nos. 8,735,553, 9,217,034, 9,834,606, 9,988,450, 10,519,235 and 11,186,637. U.S. Pat. Nos. 8,735,553, 9,217,034, 9,834,606, 9,988,450, 10,519,235 and 11,186,637 are incorporated by reference herein in their entireties, and in particular in respect to description therein of anti-PD-1 antibodies.

In some embodiments, the anti-PD-1 antibody is selected from one of the following: nivolumab (MDX 1106, BMS 936558, ONO-4538, Opdivo®) described in U.S. Pat. No. 8,008,449B2, a fully human IgG4 antibody that binds to and blocks the activation of PD-1 by its ligands PD-L1 and PD-L2; pembrolizumab (lambrolizumab, MK-3475 or SCH 900475, Keytruda®) disclosed in U.S. Pat. No. 8,168,757B2, a humanized monoclonal IgG4 antibody against PD-1; pidilizumab (CT-011), a humanized antibody that binds PD-1; AMP-224, a fusion protein of B7-DC; an antibody Fc portion; BMS-936559 (MDX-1105-01) for PD-L1 (B37-H1) blockade for PD-1 blockade. In some embodiments, the anti-PD-1 antibody is pembrolizumab, nivolumab or pidilizumab.

In some embodiments, the anti-PD-1 antibody is an antibody which comprises a heavy chain variable region (Vh) and a light chain variable region (V) comprising any set of the complementarity determining regions (CDRs) listed below in Table 3 (with three Vh CDRs and three Vl CDRs):

TABLE 3

| | |
|---|---|
| a) mu317 | CDR-H1, CDR-H2 and CDR-H3 (SEQ ID NOs: 11, 12, 13, respectively); and |
| | CDR-L1, CDR-L2 and CDR-L3 (SEQ ID NOs: 14, 15, 16, respectively); |
| b) mu326 | CDR-H1, CDR-H2 and CDR-H3 (SEQ ID NOs: 17, 18, 19, respectively); and |
| | CDR-L1, CDR-L2 and CDR-L3 (SEQ ID NOs: 20, 21, 22, respectively); |
| c) 317-4B6 | CDR-H1, CDR-H2 and CDR-H3 (SEQ ID NOs: 31, 32, 33, respectively); and |
| | CDR-L1, CDR-L2 and CDR-L3 (SEQ ID NOs: 34, 35, 36, respectively); |
| d) 326-4A3 | CDR-H1, CDR-H2 and CDR-H3 (SEQ ID NOs: 37, 38, 39, respectively); and |
| | CDR-L1, CDR--L2 and CDR-L3 (SEQ ID NOs: 40, 41, 42, respectively); |
| e) 317-1H | CDR-H1, CDR-H2 and CDR-H3 (SEQ ID NOs: 11, 59, 13, respectively); and |
| | CDR-L1, CDR-L2 and CDR-L3 (SEQ ID NOs: 14, 15, 16, respectively); |
| f) 317-4B2 | CDR-HL CDR-H2 and CDR-H3 (SEQ ID NOs: 11, 60, 13, respectively); and |
| | CDR-L1, CDR-L2 and CDR-L3 (SEQ ID NOs: 61, 15, 16, respectively); |
| g) 317-4B5 | CDR-HI, CDR-H2 and CDR-H3 (SEQ ID NOs: 11, 60, 13, respectively); and |
| | CDR-L1, CDR-L2 and CDR-L3 (SEQ ID NOs: 61, 15, 16, respectively); |
| h) 317-4B6 | CDR-H1, CDR-H2 and CDR-H3 (SEQ ID NOs: 11, 32, 13, respectively); and |
| | CDR-L1, CDR-L2 and CDR-L3 (SEQ ID NOs: 61, 15, 16, respectively); |
| i) 326-1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| j) 326-3B1 | CDR-H1, CDR-H2 and CDR-H3 (SEQ ID NOs: 17, 62, 19, respectively); and |
| | CDR-L1, CDR-L2 and CDR-L3 (SEQ ID NOs: 20, 21, 22, respectively); |
| or k) 326-3G1 | CDR-H1, CDR-H2 and CDR-H3 (SEQ ID NOs: 17, 62, 19, respectively); and |
| | CDR-L1, CDR-I 2 and CDR-L3 (SEQ ID NOs: 20, 21, 22, respectively). |

In some embodiments, the anti-PD-1 antibody comprises a heavy chain variable region (Vh) comprising complementarity determining regions (CDRs) CDR-H1, CDR-H2 and CDR-H3 of SEQ ID NOs: 31, 32, 33, respectively, and a light chain variable region (Vl) comprising CDRs CDR-L1, CDR-L2 and CDR-L3 of SEQ ID NOs: 34, 35, 36, respectively. In some embodiments, the ani-PD-1 antibody comprises Vh and V CDRs of the 317-436 antibody described herein.

In some embodiments, the anti-PD-1 antibody is an antibody which comprises a heavy chain variable region (Vh) and a light chain variable region (Vl) that contain any combinations of the CDRs listed below (with three Vh CDRs and three Vl CDRs) as shown in Table 4:

TABLE 4

| | |
|---|---|
| (a) | CDR-H1 (SEQ ID NO 31), CDR-H2 (SEQ ID NO 12, 32, 59 or |
| | 60) and CDR-H3 (SEQ ID NO 33), |
| | CDR-L1 ( SEQ ID NO 14, 34 or 61), CDR-L2 (SEQ ID NO 35) |
| | and CDR-L3 (SEQ ID NO 36); or |
| (b) | CDR-H1 (SEQ ID NO 37), CDR-H2 (SEQ ID NO 18, 38 or 62) |
| | and CDR-H3 (SEQ ID NO 39), |
| | CDR-L1 (SEQ ID NO 40), CDR-L2 (SEQ ID NO 41) and |
| | CDR-L3 (SEQ ID NO 42). |

In some embodiments, the anti-PD-1 monoclonal antibody is an antibody which comprises a heavy chain variable region (Vh) and a light chain variable region (Vl) comprising:

TABLE 5

a) mu317 (SEQ ID NOs: 4 and 6,
respectively);
b) mu326 (SEQ ID NOs: 8 and 10,
respectively);
c) 317-4B6 (SEQ ID NOs: 24 and 26,
respectively);
d) 326-4A3 (SEQ ID NOs: 28 and 30,
respectively);
e) 317-4B2 (SEQ ID NOs: 43 and 44,
respectively);
f) 317-4B5 (SEQ ID NOs: 45 and 46,
respectively);
g) 317-1 (SEQ ID NOs: 48 and 50,
respectively);
h) 326-3B1 (SEQ ID NOs: 51 and 52,
respectively);
i) 326-3GI (SEQ ID NOs: 53 and 54,
respectively);
j) 326-1 (SEQ ID NOs: 56 and 58,
respectively);
k) 317-3A1 (SEQ ID NOs: 64 and 26,
respectively);
1) 317-3C1 (SEQ ID NOs: 65 and 26,
respectively);
m) 317-3E1 (SEQ ID NOs: 66 and 26,
respectively);
n) 317-3F1 (SEQ ID NOs: 67 and 26,
respectively);
o) 317-3G1 (SEQ ID NOs: 68 and 26,
respectively);
p) 317-3H1 (SEQ ID NOs: 69 and 26,
respectively);
q) 317-311 (SEQ ID NOs: 70 and 26,
respectively);
r) 317-4B 1 (SEQ ID NOs: 71 and 26,
respectively);
s) 317-4B3 (SEQ ID NOs: 72 and 26,
respectively);
t) 317-4B4 (SEQ ID NOs: 73 and 26,
respectively);
u) 317-4A2 (SEQ ID NOs: 74 and 26,
respectively);
v) 326-3 A 1 (SEQ ID NOs: 75 and 30,
respectively);
w) 326-3C1 (SEQ ID NOs: 76 and 30,
respectively);
x) 326-3D1 (SEQ ID NOs: 77 and 30,
respectively);
y) 326-3E1 (SEQ ID NOs: 78 and 30,
respectively);
z) 326-3F1 (SEQ ID NOs: 79 and 30,
respectively);
aa) 326-3B N55D (SEQ ID NOs: 80 and 30,
respectively);
ab) 326-4A1 (SEQ ID NOs: 28 and 81,
respectively); or
ac) 326-4A2 (SEQ ID NOs: 28 and 82,
respectively).

In some embodiments, the anti-PD-1 antibody comprises a heavy chain variable region (Vh) comprising the amino acid sequence of SEQ ID NO:24 and a light chain variable region (Vl) comprising the amino acid sequence of SEQ ID NO:26. In some embodiments, the anti-PD-1 antibody comprises the Vh and Vl of the 317-4B6 antibody described herein.

In some embodiments, the anti-PD-1 antibody comprises a heavy chain variable region (Vh) comprising an amino acid sequence at least 90, 91, 92, 93, 94, 95, 96, 97, 98 or 99% identical to the amino acid sequence of SEQ ID NO:24 and a light chain variable region (Vl) comprising an amino acid sequence at least 90, 91, 92, 93, 94, 95, 96, 97, 98 or 99% identical to the amino acid sequence of SEQ ID NO:26.

In some embodiments, the anti-PD-1 antibody comprises a heavy chain variable region (Vh) or light chain variable region (Vl) encoded by the cDNA of one of more of SEQ ID NO: 23, 25, 27, 29, 47, 49, 55, 57.

In some embodiments, the antibody comprises an IgG4 Fc region having a serine to proline mutation at position 228 (EU numbering system). In some embodiments, this mutation is referred to as the S228P mutation. In some embodiments, the antibody comprises an IgG4 Fc region having a mutation at one or more of positions 233, 234, 235, 265, 309, and 409 (EU numbering system). For example, in some embodiments, the antibody comprises an IgG4 region having a mutation at 228 and at least one other position, wherein the at least one other mutation results in reduced binding to one or more FcγR. In further embodiments, the antibody comprises an IgG4 region having a mutation at position 228 and at least two, at least 3, at least 4, at least 5, or at least 6 additional positions, wherein one or more of the additional mutations results in reduced binding to one or more FcγR. In some embodiments, the antibody comprises an IgG4 region having mutations at positions 234 and 235. In some embodiments, the antibody comprises an IgG4 region having mutations at positions 233, 235, and 235. In some embodiments, the antibody comprises an IgG4 region having mutations at positions 234, 235, and 265. In some embodiments, the antibody comprises an IgG4 region having mutations at positions 233, 234, 235, and 265. In some embodiments, the antibody comprises an IgG4 region having mutations at positions 234, 235, 265, and 409. In some embodiments, the antibody comprises an IgG4 region having mutations at positions 233, 234, 235, 265, and 409. In some embodiments, the antibody comprises an IgG4 region having mutations at positions 234, 235, 265, 309, and 409. In some embodiments, the antibody comprises an IgG4 region having mutations at positions 233, 234, 235, 265, 309, and 409. The mutation at position 234 may be a phenylalanine to valine substitution or a phenylalanine to alanine substitution. The mutation at position 235 may be a leucine to alanine substitution. The mutation at position 233 may be a glutamic acid to proline substitution. The mutation at position 265 may be a aspartic acid to valine substitution or an aspartic acid to threonine substitution. The mutation at position 309 may be a leucine to valine substitution. The mutation at position 409 may be an arginine to a lysine, threonine, or methionine substitution.

In some embodiments, the anti-PD-1 antibody is an antibody which comprises a IgG4 heavy chain effector or constant domain comprising any of SEQ ID NOs: 83-88 or 91-106. In some embodiments, the anti-PD-1 antibody comprises an IgG constant region comprising any one of the amino acid sequences: SEQ ID NO:83, SEQ ID NO:84, SEQ ID NO:85, SEQ ID NO:86, SEQ ID NO:87 and SEQ ID NO:88. In some embodiments, the anti-PD-1 antibody comprises an IgG constant region comprising any one of the amino acid sequences: SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105 and SEQ ID NO:106. In some embodiments, the anti-PD-1 antibody comprises an IgG constant region comprising SEQ ID NO:83. In some embodiments, the anti-PD-1 antibody comprises an IgG constant region comprising SEQ ID NO:84. In some embodiments, the anti-PD-1 antibody comprises an IgG constant region comprising SEQ ID NO:85. In some embodiments, the anti-PD-1 antibody comprises an IgG constant region comprising SEQ ID NO:86. In some embodiments, the anti-PD-1 antibody comprises an IgG constant region comprising SEQ ID NO:87. In some embodiments, the anti-PD-1 antibody comprises an IgG constant region comprising SEQ ID NO:88. In some embodiments, the anti-PD-1 antibody comprises an IgG constant region comprising SEQ ID NO:91. In some embodiments, the anti-PD-1 antibody comprises an IgG constant region comprising SEQ ID NO:92. In some embodiments, the anti-PD-1 antibody comprises an IgG constant region comprising SEQ ID NO:93. In some embodiments, the anti-PD-1 antibody comprises an IgG constant region comprising SEQ ID NO:94. In some embodiments, the anti-PD-1 antibody comprises an IgG constant region comprising SEQ ID NO:95. In some embodiments, the anti-PD-1 antibody comprises an IgG constant region comprising SEQ ID NO:96. In some embodiments, the anti-PD-1 antibody comprises an IgG constant region comprising SEQ ID NO:97. In some embodiments, the anti-PD-1 antibody comprises an IgG constant region comprising SEQ ID NO:98. In some embodiments, the anti-PD-1 antibody comprises an IgG constant region comprising SEQ ID NO:99. In some embodiments, the anti-PD-1 antibody comprises an IgG constant region comprising SEQ ID NO:100. In some embodiments, the anti-PD-1 antibody comprises an IgG constant region comprising SEQ ID NO:101. In some embodiments, the anti-PD-1 antibody comprises an IgG constant region comprising SEQ ID NO:102. In some embodiments, the anti-PD-1 antibody comprises an IgG constant region comprising SEQ ID NO:103. In some embodiments, the anti-PD-1 antibody comprises an IgG constant region comprising SEQ ID NO:104. In some embodiments, the anti-PD-1 antibody comprises an IgG constant region comprising SEQ ID NO:105. In some embodiments, the anti-PD-1 antibody comprises an IgG constant region comprising SEQ ID NO:106.

In some embodiments, the anti-PD-1 antibody is an antibody which contains a F(ab) or F(ab)2 comprising a heavy chain variable region (Vh), a light chain variable region (Vl) and a IgG4 heavy chain effector or constant domain.

In some embodiments, the anti-PD-1 antibody is an antibody which comprises a heavy chain variable region (Vh) and a light chain variable region (Vl), and a IgG4 heavy chain effector or constant domain comprising SEQ ID NOs: 87 or 88, wherein the heavy chain variable region (Vh) and the light chain variable region (Vl) comprise:

TABLE 6

a) mu317 (SEQ ID NOs: 4 and 6,
respectively);
b) mu326 (SEQ ID NOs: 8 and 10,
respectively);
c) 317-4B6 (SEQ ID NOs: 24 and 26,
respectively);
d) 326-4A3 (SEQ ID NOs: 28 and 30,
respectively);
e) 317-4B2 (SEQ ID NOs: 43 and 44,
respectively);
f) 317-4B5 (SEQ ID NOs: 45 and 46,
respectively);
g) 317-1 (SEQ ID NOs: 48 and 50,
respectively);
h) 326-3B1 (SEQ ID NOs: 51 and 52,
respectively);
i) 326-3GI (SEQ ID NOs: 53 and 54,
respectively);
j) 326-1 (SEQ ID NOs: 56 and 58,
respectively);
k) 317-3A1 (SEQ ID NOs: 64 and 26,
respectively);
1) 317-3C1 (SEQ ID NOs: 65 and 26,
respectively);
m) 317-3E1 (SEQ ID NOs: 66 and 26,
respectively);
n) 317-3F1 (SEQ ID NOs: 67 and 26,
respectively);
o) 317-3G1 (SEQ ID NOs: 68 and 26,
respectively);
p) 317-3H1 (SEQ ID NOs: 69 and 26,
respectively);
q) 317-311 (SEQ ID NOs: 70 and 26,
respectively);
r) 317-4B 1 (SEQ ID NOs: 71 and 26,
respectively);
S) 317-4B3 (SEQ ID NOs: 72 and 26,
respectively);
t) 317-4B4 (SEQ ID NOs: 73 and 26,
respectively);
u) 317-4A2 (SEQ ID NOs: 74 and 26,
respectively);
v) 326-3 A 1 (SEQ ID NOs: 75 and 30,
respectively);
w) 326-3C1 (SEQ ID NOs: 76 and 30,
respectively);
x) 326-3D1 (SEQ ID NOs: 77 and 30,
respectively);
y) 326-3E1 (SEQ ID NOs: 78 and 30,
respectively);
z) 326-3F1 (SEQ ID NOs: 79 and 30,
respectively);
aa) 326-3B N55D (SEQ ID NOs: 80 and 30,
respectively);
ab) 326-4A1 (SEQ ID NOs: 28 and 81,
respectively); or
ac) 326-4A2 (SEQ ID NOs: 28 and 82,
respectively).

In some embodiments, the anti-PD-1 antibody is an antibody which comprises a heavy chain variable region (Vh) and a light chain variable region (Vl), and a IgG4 heavy chain effector or constant domain comprising SEQ ID NOs: 88.

In some embodiments, the anti-PD-1 antibody is an antibody which comprises a heavy chain CDR-H1, CDR-H2, and CDR-H3 according to SEQ ID NOs: 11, 32, and 13, respectively; and a light chain CDR-L1, CDR-L2, and CDR-L3 according to SEQ ID NOs: 61, 15, and 16, respectively.

In some embodiments, the anti-PD-1 antibody is an antibody which comprises a heavy chain CDR-H1, CDR-H2 and CDR-H3 according to SEQ ID NOs: 31, 32, 33, respectively; and a light chain CDR-L1, CDR-L2 and CDR-L3 according to SEQ ID NOs: 34, 35, 36, respectively. In some embodiments, the anti-PD-1 monoclonal antibody is an antibody which comprises a heavy chain variable region (Vh) and a light chain variable region (Vl) comprising SEQ ID NO: 24 and SEQ ID NO: 26, respectively.

In some embodiments, the anti-PD-1 antibody is an antibody which comprises a heavy chain variable region (Vh) and a light chain variable region (Vl) (comprising SEQ ID NO: 24 and SEQ ID NO: 26, respectively) and a IgG4 heavy chain effector or constant domain (comprising SEQ ID NO: 88). In some embodiments, the anti-PD-1 antibody specifically binds to PD-1, such as to PD-1 residues including K45 and 193; or, 193, L95 and P97, and inhibits PD-1-medicated cellular signaling and activities in immune cells, with the antibody binding to a set of amino acid residues required for its ligand binding.

In any of the above-described embodiments, the anti-PD-1 antibody can be monoclonal. In some embodiments, the antibody is monoclonal and humanized. In some embodiments, the antibody is monoclonal, humanized, IgG4 antibody. In some embodiments, the anti-PD-1 antibody is a monoclonal antibody or a fragment thereof, comprising a heavy chain variable region (Vh) amino acid sequence of SEQ ID NO: 24, a light chain variable region (Vl) amino acid sequence of SEQ ID NO: 26, and an IgG4 constant domain amino acid sequence of SEQ ID NO: 88. In some embodiments, the anti-PD-1 antibody is Tislelizumab. The sequence of Tislelizumab is known in the art.

In some embodiments, the present disclosure provides for anti-PD1 antibodies and antigen-binding fragments thereof having the sequences provided in the Table below. In some embodiments, the anti-PD1 antibody or antigen-binding fragment thereof specifically binds human PD1 and comprises the six CDRs provided in the Table below. In some embodiments, the ani-PD1 antibody or antigen-binding fragment thereof specifically binds human PD1 and comprises a heavy chain variable region and a light chain variable region comprising the sequences provided in the Table below. In some embodiments, the anti-PD1 antibody comprises an IgG4 constant domain comprising any of the sequences provided below. In some embodiments, the IgG4 constant domain comprises one of the last two sequences provided in Table 7 below.

TABLE 7
Sequences of anti-PD1 antibody

| Domain | SEQ ID NO: | Amino Acid Sequence |
|---|---|---|
| CDR-H1 | SEQ ID NO: 31 | GFSLTSYGVH |
| CDR-H2 | SEQ ID NO: 32 | VIYADGSTNYNPSLKS |
| CDR-H3 | SEQ ID NO: 33 | ARAYGNYWYIDV |
| CDR-L1 | SEQ ID NO: 34 | KSSESVSNDVA |
| CDR-L2 | SEQ ID NO: 35 | YAFHRFT |
| CDR-L3 | SEQ ID NO: 36 | HQAYSSPYT |
| Vh | SEQ ID NO: 24 | QVQLQESGPGLVKPSETLSLTCTVSGFSLTSYGVH |
| | | WIRQPPGKGLEWIGVIYADGSTNYNPSLKSRVTISK |
| | | DTSKNQVSLKLSSVTAADTAVYYCARAYGNYWYI |
| | | DVWGQGTTVTVSS |
| Vl | SEQ ID NO: 26 | DIVMTQSPDSLAVSLGERATINCKSSESVSNDVAW |
| | | YQQKPGQPPKLLINYAFHRFTGVPDRFSGSGYGTD |
| | | FTLTISSLQAEDVAVYYCHQAYSSPYTFGQGTKLEI |
| | | K |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPEFEGGPSVFLFPPKPKDTLMISRTPEVTCV |
| | | VVDVSQEDPEVQFNWYVDGVEVHNAKTKPREEQ |
| | | FNSTYRVVSVLTVLHQDWLNGKEYK |
| | | CKVSNKGLPSSIEKTISKAKGQPREPQVYTLPPSQE |
| | | EMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPPVAGGPSVFLFPPKPKDTLMISRTPEVTCV |
| | | VVDVSQEDPEVQFNWYVDGVEVHNAKTKPREEQ |
| | | FNSTYRVVSVLTVLHQDWLNGKEYK |
| | | CKVSNKGLPSSIEKTISKAKGQPREPQVYTLPPSQE |
| | | EMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPPVAGGPSVFLFPPKPKDTLMISRTPEVTCV |
| | | VVAVSQEDPEVQFNWYVDGVEVHNAKTKPREEQ |
| | | FNSTYRVVSVLTVLHQDWLNGKEYK |
| | | CKVSNKGLPSSIEKTISKAKGQPREPQVYTLPPSQE |
| | | EMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPPVAGGPSVFLFPPKPKDTLMISRTPEVTCV |
| | | VVTVSQEDPEVQFNWYVDGVEVHNAKTKPREEQ |
| | | FNSTYRVVSVLTVLHQDWLNGKEYK |
| | | CKVSNKGLPSSIEKTISKAKGQPREPQVYTLPPSQE |
| | | EMTKNQVSLTCLVKGFYPSDIAVEWESNGQPENN |
| | | YKTTPPVLDSDGSFFLYSRLTVDKSRWQEGNVFSC |
| | | SVMHEALHNHYTQKSLSLSLGK |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPPVAGGPSVFLFPPKPKDTLMISRTPEVTCV |
| | | VVAVSQEDPEVQFNWYVDGVEVHNAKTKPREEQ |
| | | FNSTYRVVSVLTVLHQDWLNGKEYK |
| | | CKVSNKGLPSSIEKTISKAKGQPREPQVYTLPPSQE |
| | | EMTKNQVSLTCLVKGFYPSDIAVEWESNGQPENN |
| | | YKTTPPVLDSDGSFFLYSKLTVDKSRWQEGNVFSC |
| | | SVMHEALHNHYTQKSLSLSLGK |
| IgG4 | | ASTKGPSVFPLAPCSRSTSESTAALGCLVKDYFPEP |
| constant | | VTVSWNSGALTSGVHTFPAVLQSSGLYSLSSVVTV |
| domain | | PSSSLGTKTYTCNVDHKPSNTKVDKRVESKYGPPC |
| | | PPCPAPPVAGGPSVFLFPPKPKDTLMISRTPEVTCV |
| | | VVAVSQEDPEVQFNWYVDGVEVHNAKTKPREEQ |
| | | FNSTYRVVSVLTVVHQDWLNGKEYKCKVSNKGL |
| | | PSSIEKTISKAKGQPREPQVYTLPPSQEEMTKNQVS |
| | | LTCLVKGFYPSDIAVEWESNGQPENNYKTTPPVLD |
| | | SDGSFFLYSKLTVDKSRWQEGNVFSCSVMHEALH |
| | | NHYTQKSLSLSLGK |

In some embodiments, the anti-PD1 antibodies or antigen-binding fragments thereof include amino acids that have been mutated, yet have at least 80%, 85%, 90%, 95%, 96%, 97%, 98% or 99% percent identity in the CDR regions with the CDR regions depicted in the sequences described herein, such as in the Table above. In some aspects, it includes mutant amino acid sequences wherein no more than 1, 2, 3, 4 or 5 amino acids have been mutated in the CDR regions when compared with the CDRs disclosed herein.

In some embodiments, the anti-PD1 antibodies include those where the amino acids or nucleic acids encoding the amino acids have been mutated; yet have at least 80%, 85%, 90%, 95%, 96%, 97%, 98% or 99% percent identity to the sequences described herein (e.g., in the heavy and light chain variable regions and/or IgG constant regions), such as in the Table above. In some aspects, it includes mutant amino acid sequences wherein no more than 1, 2, 3, 4 or 5 amino acids have been mutated in the heavy and light chain variable regions when compared with the heavy and light chain variable regions in the sequence disclosed herein, while retaining substantially the same therapeutic activity.

The anti-PD-1 antibodies and antigen-binding fragments thereof described herein can be made by any method known in the art and/or described herein. Methods of making monoclonal antibodies are well known in the art. See e.g., Harlow E and Lane D, Antibodies: A Laboratory Manual (Cold Spring Harbor Press, 2^{nd} ed. 1988); Hammerling G J et al., in: Monoclonal Antibodies and T-Cell Hybridomas 563 (Elsevier, NY, 1981); Kohler G and Milstein C, 1975, Nature 256:495; Goding J W (Ed), Monoclonal Antibodies: Principles and Practice, pp. 59-103 (Academic Press, 1986). Methods of making humanized antibodies are also well known in the art. See, e.g., Padlan E A (1991) Mol Immunol 28(4/5): 489-498; Studnicka G M et al, (1994) Prot Engineering 7(6): 805-814; and Roguska M A et al, (1994) PNAS 91: 969-973; Tan P et al, (2002) J Immunol 169: 1119-25; Caldas C et al, (2000) Protein Eng. 13(5): 353-60; Morea V et al, (2000), Methods 20(3): 267-79; Baca M et al, (1997) J Biol Chem 272(16): 10678-84; Roguska M A et al, (1996) Protein Eng 9(10): 895 904; Couto J R et al, (1995) Cancer Res. 55 (23 Supp): 5973s-5977s; Couto J R et al, (1995) Cancer Res 55(8): 1717-22; Sandhu J S (1994) Gene 150(2): 409-10; Pedersen J T et al, (1994) J Mol Biol 235(3): 959-73). Methods of making human antibodies are also known in the art. See, e.g., International Publication Nos. WO 98/46645, WO 98/50433, WO 98/24893, WO 98/16654, WO 96/34096, WO 96/33735, and WO 91/10741.

The anti-PD1 monoclonal antibodies and antibody fragments thereof may be prepared in accordance with the disclosure of WO2015/035606A1 or US 2015-0315274, the entire disclosures of which are incorporated by reference herein.

### Anti-PD-1 Antibody Dosing and Administration

In some embodiments, the anti-PD-1 antibody (e.g., any of the antibodies described herein) is administered by any suitable means. In some embodiments, the anti-PD-1 antibody is administered parenterally. In some embodiments, the anti-PD-1 antibody is administered intravenously (IV). In some embodiments, the anti-PD-1 antibody is administered by IV infusion. In some embodiments, the anti-PD-1 antibody is infused over 60 minutes or more. In some embodiments, the anti-PD-1 antibody is infused over 30 minutes or more. In some embodiments, the anti-PD-1 antibody is administered subcutaneously. In some embodiments, the anti-PD-1 antibody is administered intramuscularly. In some embodiments, the anti-PD-1 antibody is administered peritoneally.

In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered parenterally. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered intravenously (IV). In some embodiments, Tislelizumab is administered by IV infusion. In some embodiments, the IV infusion is over 60 minutes or more. In some embodiments, the IV infusion is over 30 minutes or more.

In some embodiments, the anti-PD-1 antibody (e.g., any of the antibodies described herein) is administered at a fixed dose. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered at a fixed dose.

In some embodiments, the anti-PD-1 antibody is administered at a dose of 200 mg or about 200 mg. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered at a dose of 200 mg or about 200 mg. In some embodiments, Tislelizumab is administered at a dose of 200 mg. In some embodiments, Tislelizumab is administered at dose from about 50 to 800 mg, optionally wherein the anti-PD-1 antibody is administered at 100 mg, 150 mg, 200, mg, 250 mg, 300 mg, 350 mg, 400 mg, 450 mg, 500 mg, 550 mg, 600 mg, 650 mg, 700 mg, 750 mg, or 800 mg. In some embodiments, the PD-1 antibody is administered every week, every 2 weeks, every 3 weeks, every 4 weeks, every 5 weeks, or every 6 weeks, preferably wherein the anti-PD-1 antibody is administered at 150 mg every two weeks, 300 mg every four weeks, or 400 mg every six weeks.

In some embodiments, the anti-PD-1 antibody is administered at a dose from about 2 mg/kg to about 200 mg/kg (and any value in between). In some embodiments, the anti-PD-1 antibody is administered at a dose from about 2 mg/kg to about 5 mg/kg (e.g., 2 mg/kg to 5 mg/kg). In some embodiments, the anti-PD-1 antibody is administered at a dose of 2 mg/kg, 2.5 mg/kg, 3 mg/kg, 3.5 mg/kg, 4 mg/kg, 4.5 mg/kg, or 5 mg/kg (or any value in between these values). In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered at a dose from about 2 mg/kg to about 5 mg/kg (e.g., 2 mg/kg to 5 mg/kg). In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered at a dose at a dose of 2 mg/kg, 2.5 mg/kg, 3 mg/kg, 3.5 mg/kg, 4 mg/kg, 4.5 mg/kg, or 5 mg/kg (or any value in between these values). In some embodiments, Tislelizumab is administered at a dose of 2 mg/kg to 5 mg/kg.

In some embodiments, the anti-PD-1 antibody is administered at a dosage of about 2 mg/kg Q3W to about 200 mg/kg once every three weeks (Q3W). In some embodiments, the anti-PD-1 antibody is administered at a dosage of about 2 mg/kg Q3W, 5 mg/kg Q3W or 200 mg flat Q3W.

In some embodiments, the anti-PD-1 antibody is administered once every three weeks. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered once every three weeks.

In some embodiments, the anti-PD-1 antibody is administered once every two weeks. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered once every two weeks.

In some embodiments, the anti-PD-1 antibody is administered once every three weeks for 4 cycles or at least 4 cycles. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered once every three weeks for 4 cycles or at least 4 cycles.

In some embodiments, the anti-PD-1 antibody is administered in one treatment cycle. In some embodiments, the anti-PD-1 antibody is administered in two treatment cycles. In some embodiments, the anti-PD-1 antibody is administered in three treatment cycles. In some embodiments, the anti-PD-1 antibody is administered in four treatment cycles. In some embodiments, the anti-PD-1 antibody is administered in five treatment cycles. In some embodiments, the anti-PD-1 antibody is administered in six treatment cycles. In some embodiments, the anti-PD-1 antibody is administered in seven treatment cycles. In some embodiments, the anti-PD-1 antibody is administered in eight treatment cycles. In some embodiments, the anti-PD-1 antibody is administered in nine treatment cycles. In some embodiments, the anti-PD-1 antibody is administered in ten treatment cycles. In some embodiments, the anti-PD-1 antibody is administered in eleven treatment cycles. In some embodiments, the anti-PD-1 antibody is administered in twelve treatment cycles. In some embodiments, the anti-PD-1 antibody is administered in thirteen treatment cycles. In some embodiments, the anti-PD-1 antibody is administered in fourteen treatment cycles. In some embodiments, the anti-PD-1 antibody is administered in fifteen treatment cycles. In some embodiments, a treatment cycle is three weeks.

In some embodiments, the anti-PD-1 antibody is administered on Day 1 of the cycle (e.g., Day 1 of 21 day or 3 week cycle).

In some embodiments, the anti-PD-1 antibody is IV infused over 60 minutes or more. In some embodiments, the anti-PD-1 antibody is IV infused over 60 minutes or more in cycle 1, and IV infused over 30 minutes or about 30 minutes in subsequent cycles (e.g., cycles 2 to 4, cycles 2 to 5, or any number of cycles after the first cycle). In some embodiments, the anti-PD-1 antibody is IV infused over 60 minutes or more in cycle 1, and IV infused over less than 60 minutes in subsequent cycles (e.g., cycles 2 to 4, cycles 2 to 5, or any number of cycles after the first cycle). In some embodiments, the infusion time for the anti-PD-1 antibody is reduced from 60 minutes to 30 minutes if, and only if, the 60 minute infusion time is well tolerated by the treated subject.

In some embodiments, the anti-PD-1 antibody is administered once every three weeks (Q3W) for 3 months or at least 3 months. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered once every three weeks (Q3W) for 3 months or at least 3 months. In some embodiments, the anti-PD-1 antibody is administered once every three weeks (Q3W) for at least 4, 5, 6, 7, 8, 9, 10, 11, 12, 18 or 24 months (or more). In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered once every three weeks (Q3W) for at least 4, 5, 6, 7, 8, 9, 10, 11, 12, 18 or 24 months (or more).

In some embodiments, the anti-PD-1 antibody is administered in an induction treatment period. In some embodiments, the anti-PD-1 antibody is administered in a maintenance treatment period. In some embodiments, the induction treatment period is followed by the maintenance treatment period. In some embodiments, the dose of anti-PD-1 antibody is the same in the maintenance period as in the induction period. In some embodiments, the dose of anti-PD-1 antibody in the induction period is 200 mg and the dose in the maintenance period is 200 mg.

In some embodiments, the dosage and frequency of administration is the same in the induction and maintenance periods. In some embodiments, the induction treatment period comprises administering an anti-PD-1 antibody once every 3 weeks. In some embodiments, the induction treatment period comprises four three-week cycles. In some embodiments, the maintenance treatment period comprises administering an anti-PD-1 antibody once every 3 weeks. In some embodiments, the maintenance treatment period comprises one three-week cycle. In some embodiments, the maintenance treatment period comprises two three-week cycles. In some embodiments, the maintenance treatment period comprises three three-week cycles. In some embodiments, the maintenance treatment period comprises four three-week cycles. In some embodiments, the maintenance treatment period comprises five three-week cycles. In some embodiments, the maintenance treatment period comprises six three-week cycles. In some embodiments, the maintenance treatment period comprises seven three-week cycles. In some embodiments, the maintenance treatment period comprises eight three-week cycles. In some embodiments, the maintenance treatment period comprises nine three-week cycles. In some embodiments, the maintenance treatment period comprises ten three-week cycles.

In some embodiments, the anti-PD-1 antibody is administered intravenously at a fixed dose of 200 mg. In some embodiments, the anti-PD-1 antibody is administered intravenously at a fixed dose of 200 mg once every three weeks. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered intravenously at a fixed dose of 200 mg. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered intravenously at a fixed dose of 200 mg once every three weeks. In some embodiments, the IV administration is by IV infusion.

In some embodiments, the anti-PD-1 antibody is administered intravenously at a dose of 2 mg/kg to 5 mg/kg (e.g., 2 mg/kg or 5 mg/kg). In some embodiments, the anti-PD-1 antibody is administered intravenously at a dose of 2 mg/kg to 5 mg/kg (e.g., 2 mg/kg or 5 mg/kg) once every three weeks. In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered intravenously at a dose of 2 mg/kg to 5 mg/kg (e.g., 2 mg/kg or 5 mg/kg). In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered intravenously at a dose of 2 mg/kg to 5 mg/kg (e.g., 2 mg/kg or 5 mg/kg) every three weeks. In some embodiments, the IV administration is by IV infusion.

In some embodiments, an anti-PD-1 antibody (e.g., Tislelizumab) is administered at 200 mg in an induction period, wherein the induction period comprises four cycles, wherein each cycle is 3 weeks. In some embodiments, an anti-PD-1 antibody (e.g., Tiselizumab) is administered at 200 mg in an induction period, wherein the induction period comprises four cycles, wherein each cycle is 3 weeks, and wherein the antibody is administered on Day 1 of each cycle.

In some embodiments, an anti-PD-1 antibody is administered at 200 mg in: (a) an induction period, wherein the induction period comprises four cycles, wherein each cycle is 3 weeks, and wherein the antibody is administered on Day 1 of each cycle, and (b) a maintenance period, wherein the maintenance period comprises at least one cycle, wherein each cycle is 3 weeks, and wherein the antibody is administered on Day 1 of each cycle.

In some embodiments, an anti-PD-1 antibody comprising the CDRs of the 317-4B6 antibody described herein, the Vh of SEQ ID NO:24, and/or the Vl of SEQ ID NO:26 (e.g., Tislelizumab) is administered at 200 mg in: (a) an induction period, wherein the induction period comprises four cycles, wherein each cycle is 3 weeks, and wherein the antibody is administered on Day 1 of each cycle, and (b) a maintenance period, wherein the maintenance period comprises at least one cycle, wherein each cycle is 3 weeks, and wherein the antibody is administered on Day 1 of each cycle.

In some embodiments, the dosage and treatment regimens described herein are for administration to human subjects.

### Anti-Cancer Agents Chemotherapeutic Drugs

In some embodiments, a subject described herein is administered an anti-cancer agent. The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents, including cellular therapies.

In some embodiments, the anti-PD-1 antibodies and antigen-binding fragments of the present disclosure are used in combination with one or more anti-cancer agents. In some embodiments, a subject described herein is administered an anti-PD-1 antibody and further administered an anti-cancer agent described herein.

In some embodiments, the anti-PD-1 antibodies of the present disclosure are used in combination with one or more chemotherapeutic agents or drugs (e.g., one, two or three chemotherapeutic agents or drugs). In some embodiments, the treatment methods described herein comprise administration to a subject (e.g., a human) of an anti-PD-1 antibody of the present disclosure and one or more chemotherapeutic drugs (e.g., one, two or three chemotherapeutic drugs).

Classes of chemotherapeutic agents that can be used include, but are not limited to: microtubule inhibitors, e.g., taxanes (paclitaxel, docetaxel), and vinca alkaloids (vincristine, vinblastine, vinorelbine); antimetabolites (5-fluorouracil, capecitabine, gemcitabine), and antifolates (methotrexate, pemetrexed); kinase inhibitors (crizotinib, erlotinib, sunitinib); topoisomerase inhibitors (deruxtecan, topotecan, irinotecan, anthracyclines, etoposide); cell-cycle independent drugs, e.g., platinum compounds and analogues (oxaliplatin), triazenes, alkylating agents (cyclophosphamide), spindle poison plant alkaloids (doxorubicin), cytotoxic/antitumor antibiotics (bleomycin, mithramycin, mitomycin), photosensitizers, anti-estrogens and selective estrogen receptor modulators (SERMs), anti-progesterones, estrogen receptor down-regulators (ERDs), estrogen receptor antagonists, luteinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Current standard of care (SOC) treatments for certain cancers, in the early-line setting include chemotherapy based on fluoropyrimidine, oxaliplatin, and irinotecan used in combination or sequentially, with option for monoclonal antibodies targeting vascular endothelial growth factor (VEGF) (e.g., bevacizumab, ziv-aflibercept) or its receptors (e.g., ramucirumab), and in patients with Ras wild type tumors, monoclonal antibodies targeting the epidermal growth factor (EGF) receptor (e.g., cetuximab, panitumumab). Treatment options for heavily pre-treated patients beyond the second-line setting are especially limited and associated toxicities can be severe.

In some embodiments, the chemotherapeutic agent is selected from one or more of the following: paclitaxel or a paclitaxel agent; (e.g., Abraxane®), docetaxel; carboplatin; topotecan; deruxtecan; cisplatin; irinotecan, doxorubicin, lenalidomide, 5-azacytidine, ifosfamide, oxaliplatin, pemetrexed disodium, cyclophosphamide, etoposide, decitabine, fludarabine, vincristine, bendamustine, chlorambucil, busulfan, gemcitabine, melphalan, pentostatin, mitoxantrone, and pemetrexed disodium.

In some embodiments, the chemotherapeutic drug is a platinum chemotherapy.

In some embodiments, the chemotherapeutic drug is cisplatin.

In some embodiments, the chemotherapeutic drug is carboplatin.

In some embodiments, the treatment methods described herein comprise administration to a subject (e.g., a human) of an anti-PD-1 antibody of the present disclosure and either cisplatin or carboplatin. In some embodiments, it is in physician's discretion whether to administer cisplatin or carboplatin.

In some embodiments, the chemotherapeutic drug is etoposide. In some embodiments, the treatment methods described herein comprise administration to a subject (e.g., a human) of an anti-PD-1 antibody of the present disclosure and etoposide.

In some embodiments, the treatment methods described herein comprise administration to a subject (e.g., a human) of (i) an anti-PD-1 antibody of the present disclosure, (ii) either cisplatin or carboplatin, and (iii) etoposide.

The dosing of the chemotherapy drugs can be any dosing known in the art (e.g., any dose and administration regimen known in the art to be effective) or any dosing described herein.

### Cisplatin

In some embodiments, a patient described herein is administered Cisplatin in combination with an anti-PD-1 antibody described herein. In some embodiments, a patient is administered Cisplatin in combination with Tislelizumab. In some embodiments, a patient is administered Cisplatin in combination with an anti-PD-1 antibody described herein and Etoposide. In some embodiments, a patient is administered Cisplatin in combination with Tislelizumab and Etoposide.

In some embodiments, Cisplatin is administered parenterally. In some embodiments, Cisplatin is administered at a dose of about 20 mg/m² to about 100 mg/m². In some embodiments, Cisplatin is administered at about 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², or 100 mg/m² (or any value in between of these values). In some embodiments, Cisplatin is administered at about 75 mg/m². In some embodiments, Cisplatin is administered at 75 mg/m².

In some embodiments, Cisplatin is administered intravenously. In some embodiments, Cisplatin is administered as an intravenous infusion over at least 2 hours. In some embodiments, Cisplatin is administered as an intravenous infusion over 2 hours. In some embodiments, Cisplatin is administered once every week. In some embodiments, Cisplatin is administered once every 3 weeks. In some embodiments, Cisplatin is administered on Day 1 once every 3 weeks. In some embodiments, Cisplatin is administered on Day 2 once every 3 weeks. In some embodiments, Cisplatin is administered on Days 1-3 every 3 weeks. In some embodiments, Cisplatin is administered on Days 1-5 every 3 weeks.

In some embodiments, Cisplatin is administered for 1 cycle. In some embodiments, Cisplatin is administered for 2 cycles. In some embodiments, Cisplatin is administered for 3 cycles. In some embodiments, Cisplatin is administered for 4 cycles. In some embodiments, a cycle is 3 weeks.

In some embodiments, Cisplatin is administered for 4 three-week cycles.

In some embodiments, Cisplatin is administered on Day 1 of each cycle. In some embodiments, Cisplatin is administered on Day 2 of each cycle. In some embodiments, Cisplatin is administered on Day 3 of each cycle. In some embodiments, Cisplatin is administered on Day 4 of each cycle. In some embodiments, Cisplatin is administered on Day 5 of each cycle.

In some embodiments, the methods described herein comprise administration of Cisplatin once every cycle, wherein each cycle is three weeks, on Day 1 of each cycle for 4 cycles.

In some embodiments, the methods described herein comprise intravenous administration of 75 mg/m² Cisplatin on Day 1 of each 21 days cycle (and optionally wherein Cisplatin is infused over 2 hours), and optionally the administration is for 4 cycles or more.

### Carboplatin

In some embodiments, a patient described herein is administered Carboplatin in combination with an anti-PD-1 antibody described herein. In some embodiments, a patient is administered Carboplatin in combination with Tislelizumab. In some embodiments, a patient is administered Carboplatin in combination with an anti-PD-1 antibody described herein and Etoposide. In some embodiments, a patient is administered Carboplatin in combination with Tislelizumab and Etoposide.

In some embodiments, Carboplatin is administered parenterally. In some embodiments, Carboplatin is administered as a target of AUC 4 mg/mL/min to 6 mg/mL/min (and any value in between, e.g., 5 to 6 mg/mL/min). In some embodiments, Carboplatin is administered as a target of AUC 4 mg/mL/min. In some embodiments, Carboplatin is administered as a target of AUC 5 mg/mL/min. In some embodiments, Carboplatin is administered as a target of AUC 6 mg/mL/min. As used herein the term “Area under the Curve” or “AUC” defines the dose of carboplatin administered to a subject. The AUC is area under the plasma concentration-time curve. In some embodiments, a subject is administered carboplatin to achieve the above-noted AUC, e.g., AUC of 5 mg/mL/min. In some embodiments, the dosing of carboplatin is calculated according to Calvert formula dosing. A skilled artisan can calculate the amount of carboplatin necessary to achieve a given AUC in a subject. See Calvert et al. Carboplatin dosage: prospective evaluation of a simple formula based on renal function. Journal of Clinical Oncology, 1989, 7(11): 1748-56, the contents of which are incorporated by reference herein in its entirety.

In some embodiments, Carboplatin is administered intravenously. In some embodiments, Carboplatin is administered as an intravenous infusion over 15 to 60 minutes (or any value in between). In some embodiments, Carboplatin is administered as an intravenous infusion 15 minutes. In some embodiments, Carboplatin is administered as an intravenous infusion 30 minutes. In some embodiments, Carboplatin is administered as an intravenous infusion 45 minutes. In some embodiments, Carboplatin is administered as an intravenous infusion 60 minutes.

In some embodiments, Carboplatin is administered once every week. In some embodiments, Carboplatin is administered once every 3 weeks. In some embodiments, Carboplatin is administered on Day 1 once every 3 weeks.

In some embodiments, Carboplatin is administered for 1 cycle. In some embodiments, Carboplatin is administered for 2 cycles. In some embodiments, Carboplatin is administered for 3 cycles. In some embodiments, Carboplatin is administered for 4 cycles. In some embodiments, a cycle is 3 weeks.

In some embodiments, Carboplatin is administered for 4 three-week cycles. In some embodiments, Carboplatin is administered on Day 1 of each cycle.

In some embodiments, the methods described herein comprise administration of Carboplatin once every cycle, wherein each cycle is three weeks, on Day 1 of each cycle for 4 cycles.

In some embodiments, the methods described herein comprise intravenous administration of AUC5 Carboplatin on Day 1 of each 21 days cycle (and optionally wherein Carboplatin is infused over 15 to 60 minutes), and optionally the administration is for 4 cycles or more. In some embodiments, carboplatin is administered to achieve AUC of 5 mg/mL/min in accordance with Calvert formula dosing (e.g., as described herein).

### Etoposide

In some embodiments, a patient described herein is administered Etoposide in combination with an anti-PD-1 antibody described herein. In some embodiments, a patient is administered Etoposide in combination with Tislelizumab. In some embodiments, a patient is administered a combination of the anti-PD-1 antibody described herein, Carboplatin or Cisplatin, and Etoposide. In some embodiments, a patient is administered a combination of Tislelizumab, Carboplatin or Cisplatin, and Etoposide.

In some embodiments, Etoposide is administered parenterally. In some embodiments, Etoposide is administered at a dose of about 80 mg/m² to about 140 mg/m² (or any value in between). In some embodiments, Etoposide is administered at 80 mg/m². In some embodiments, Etoposide is administered at 90 mg/m². In some embodiments, Etoposide is administered at 100 mg/m². In some embodiments, Etoposide is administered at 110 mg/m². In some embodiments, Etoposide is administered at 120 mg/m². In some embodiments, Etoposide is administered at 130 mg/m². In some embodiments, Etoposide is administered at 140 mg/m².

In some embodiments, Etoposide is administered intravenously. In some embodiments, Etoposide is administered as an intravenous infusion over at least 60 minutes. In some embodiments, Etoposide is administered as an intravenous infusion over 60 minutes.

In some embodiments, Etoposide is administered once every week. In some embodiments, Etoposide is administered three times in three weeks. In some embodiments, Etoposide is administered 3 times in the first week of a 3 week (21 day) cycle. In some embodiments, Etoposide is administered on Day 1 of a 3 week (21 day) cycle. In some embodiments, Etoposide is administered on Day 2 of a 3 week (21 day) cycle. In some embodiments, Etoposide is administered on Day 3 of a 3 week (21 day) cycle. In some embodiments, Etoposide is administered on Day 4 of a 3 week (21 day) cycle. In some embodiments, Etoposide is administered on Day 5 of a 3 week (21 day) cycle. In some embodiments, Etoposide is administered on Days 1-3 every 3 weeks. In some embodiments, Etoposide is administered on Days 1-5 every 3 weeks.

In some embodiments, Etoposide is administered for 1 cycle. In some embodiments, Etoposide is administered for 2 cycles. In some embodiments, Etoposide is administered for 3 cycles. In some embodiments, Etoposide is administered for 4 cycles. In some embodiments, a cycle is 3 weeks.

In some embodiments, Etoposide is administered for 4 three-week cycles.

In some embodiments, Etoposide is administered on Day 1, Day 2 and Day 3 of each cycle. In some embodiments, Etoposide is administered on Day 1, Day 2, Day 3, Day 4 and Day 5 of each cycle.

In some embodiments, the methods described herein comprise administration of Etoposide three times every cycle, wherein each cycle is three weeks, for 4 cycles.

In some embodiments, the methods described herein comprise intravenous administration of 100 mg/m² Etoposide on Day 1 to Day 3 of each 21 days cycle (and optionally wherein Etoposide is infused over 60 minutes), and optionally the administration is for 4 cycles or more.

### Combination Therapies

The combination therapy may be administered as a simultaneous, or separate or sequential regimen. When administered sequentially, the combination may be administered in two or more administrations. The combined administration includes co-administration, using separate formulation, and consecutive administration in either order, wherein preferably there is a time period while both (or each of) active agents simultaneously exert their biological activities.

In some embodiments, the combination therapies provided herein provide a synergistic and/or statistically improved effect relative to either therapy when used as a single agent. For example, the combination therapies disclosed herein result in significant improvement in patient outcome in SCLC or extensive stage SCLC. For example, the combination therapies, in some embodiments, prolong patient survival and progression-free survival while having a manageable toxicity profile in patients suffering from SCLC or extensive stage SCLC.

Suitable dosages for any of the above co-administered agents are those presently used and may be lowered due to the combined action (synergy), such as to increase the therapeutic index or mitigate toxicity or other side-effects or consequences.

In some embodiments, the anti-PD-1 antibody and one or more chemotherapy drug (or combination treatment regimens described herein) may be further combined with any other therapy. In some embodiments, the anti-PD-1 antibody and one or more chemotherapy drug (or combination treatment regimens described herein) may be further combined with any therapy except any one or more therapies listed in the exclusion criteria described in the example section of this application. In some embodiments, the anti-PD-1 antibody and one or more chemotherapy drug (or combination treatment regimens described herein) may be further combined with, e.g., radiotherapy. In some embodiments, the anti-PD-1 antibody and one or more chemotherapy drug (or combination treatment regimens described herein) may be further combined with, e.g., a steroid or hormone therapy, optionally wherein the steroid or hormone therapy is administered to alleviate incidence of immune-mediated adverse events.

In some embodiments, the combination therapy is any combination therapy described herein, including without limitation the combination therapies, dosing, duration and regimens described in the example section of this application.

In some embodiments, the combination therapy described herein can be combined with additional therapies consistent with the inclusion criteria described in the example section of this application.

In some embodiments, the combination therapy described herein can be combined with additional therapies consistent with the exclusion criteria described in the example section of this application (i.e., wherein the therapies listed in the exclusion criteria are excluded or not selected for the combination).

In some embodiments, the additional therapy can be one or more of the following: tyrosine kinase inhibitor (e.g., EGFR inhibitor (e.g., erlotinib), multikinase inhibitor (e.g., MGCD265, RGB-286638), CD-20 targeting agent (e.g., rituximab, ofatumumab, R05072759, LFB-R603), CD52 targeting agent (e.g., alemtuzumab), prednisolone, darbepoetin alfa, lenalidomide, Bcl-2 inhibitor (e.g., oblimersen sodium), aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), EGEN-001, Polo-like kinase 1 inhibitor (e.g., BI 672).

In some embodiments, the additional therapy can be an antibody binding to another immune checkpoint molecule.

In some embodiments, the amounts of the anti-PD-1 antibody disclosed herein and the one or chemotherapy drugs, as well as their relative timings of administration, be determined by the individual needs of the patient to be treated, administration route, severity of disease or illness, dosing schedule, as well as evaluation and judgment of the designated doctor.

In some embodiments, the anti-PD-1 antibody is administered before (e.g., 60 minutes or more before) administration of one or more chemotherapy drugs. In some embodiments, the anti-PD-1 antibody is administered before (e.g., 30 minutes or more before) administration of one or more chemotherapy drugs. In some embodiments, the anti-PD-1 antibody is administered 60 minutes or more before administration of one or more chemotherapy drugs in cycle 1 and optionally cycle 2. In some embodiments, the anti-PD-1 antibody is administered 30 minutes or more before administration of one or more chemotherapy drugs in cycle 3, cycle 4, and/or any cycle subsequent to cycle 3.

In some embodiments, the dosing, frequency and/or duration of administration of the anti-PD-1 antibody and one or more chemotherapeutic agents is as described herein.

In some embodiments, the anti-PD-1 antibody and the one or more chemotherapeutic drugs described herein are administered on the same day. In some embodiments, the anti-PD-1 antibody and the one or more chemotherapeutic drugs described herein are administered at least 30 minutes apart from each other. In some embodiments, the anti-PD-1 antibody and the one or more chemotherapeutic drugs described herein are administered in any sequence. In some embodiments, the anti-PD-1 antibody is administered before the administration of the one or more chemotherapeutic drugs as described herein (e.g., at least 30 minutes before).

In some embodiments, anti-PD-1 antibody (such as an anti-PD-1 antibody comprising the CDRs of the 317-4B6 antibody described herein, the Vh of SEQ ID NO:24, and/or the Vl of SEQ ID NO:26 (e.g., Tislelizumab)) is administered intravenously at a dose of 200 mg on Day 1 of each 21 cycle; cisplatin or carboplatin is administered intravenously on Day of each 21 day cycle; and etoposide is administered intravenously on Days 1 to 3 of each 21 day cycle. In some of these embodiments, cisplatin is administered IV at 75 mg/m² (and optionally infused over 2 hours) or carboplatin is administered IV at AUC5 (and optionally infused over 15-60 minutes to achieve an AUC of 5 mg/mL/min). In some of these embodiments, etoposide is administered IV at 100 mg/m² (and optionally infused over 60 minutes). In some of these embodiments, in cycle 1, the anti-PD-1 antibody is administered 60 minutes or more before administration of cisplatin, carboplatin and/or etoposide (and optionally the anti-PD-1 antibody is infused over 60 minutes). In some of these embodiments, in cycle 2, the anti-PD-1 antibody is administered 60 minutes or more before administration of cisplatin, carboplatin and/or etoposide (and optionally the anti-PD-1 antibody is infused over 30 minutes). In some of these embodiments, in cycles 3 and 4, the anti-PD-1 antibody is administered 30 minutes or more (e.g., less than 60 minutes) before administration of cisplatin, carboplatin and/or etoposide (and optionally the anti-PD-1 antibody is infused over 30 minutes). In some of these embodiments, in subsequent cycles (after cycle 4), the anti-PD-1 antibody is administered alone (without chemotherapy), and optionally the anti-PD-1 antibody is infused over 30 minutes or more (e.g., for less than 60 minutes).

### Cancers Treated as Provided Herein

Treatment regimens for lung cancer are disclosed herein. In some embodiments, the cancer is small cell lung cancer (SCLC).

In some embodiments, the cancer is mid-stage lung cancer (e.g., SCLC). In some embodiments, the cancer is late-stage lung cancer (e.g., SCLC). In some embodiments, the cancer is locally advanced lung cancer (e.g., SCLC). In some embodiments, the cancer is advanced stage lung cancer (e.g., SCLC). In some embodiments, the cancer is extensive stage lung cancer (e.g., SCLC). In some embodiments, the lung cancer (e.g., SCLC) is unresectable. In some embodiments, the lung cancer (e.g., SCLC) is metastatic.

In some embodiments, the cancer is stage IIIA lung cancer. In some embodiments, the cancer is stage IIIB lung cancer. In some embodiments, the cancer is stage IV lung cancer.

In some embodiments, the lung cancer (e.g., SCLC) has not undergone prior treatment. In some embodiments, the lung cancer (e.g., SCLC) is extensive, advanced or metastatic and has not received any prior line therapy. In some embodiments, the lung cancer (e.g., SCLC) is extensive, advanced or metastatic and has not received prior chemotherapy (e.g., platinum-based) or immunotherapy.

In some embodiments, the lung cancer is an ES-SCLC that has not undergone prior treatment. In some embodiments, the lung cancer is ES-SCLC that has not received prior chemotherapy (e.g., platinum-based) or prior immunotherapy.

In some embodiments, the human patient has received at least one prior line chemotherapy (e.g., platinum-based systemic anticancer therapy). In some embodiments, the human patient has received at least one prior line chemotherapy (e.g., platinum-based systemic anticancer therapy) for extensive, advanced or metastatic SCLC.

In some embodiments, the lung cancer is chemotherapy-resistant. In some embodiments, the lung cancer has progressed after prior chemotherapy.

In some embodiments, the SCLC is small cell carcinoma. In some embodiments, the SCLC is combined small cell carcinoma. In some embodiments, the SCLC is of neuroendocrine (NE)-high subtype. In some embodiments, the SCLC is of neuroendocrine (NE)-low subtype.

In some embodiments, the SCLC is stage IIIA. In some embodiments, the SCLC is stage IIIB. In some embodiments, the SCLC is stage IV. In some embodiments, the cancer is extensive-stage small cell lung cancer (ES-SCLC). In some embodiments, extensive-stage cancer has spread outside of the lung in which it began or to other parts of the body. In some embodiments, the SCLC is unresectable. In some embodiments, the SCLC is locally advanced. In some embodiments, the SCLC is metastatic.

In some embodiments, the lung cancer is characterized by one or more of liver, lung, lymph node, bone, or adrenal gland metastasis. In some embodiments, the cancer is characterized by liver metastasis. In some embodiments, the cancer is characterized by lung metastasis. In some embodiments, the cancer is characterized by lymph node metastasis. In some embodiments, the cancer is characterized by bone metastasis. In some embodiments, the cancer is characterized by adrenal gland metastasis.

In some embodiments, the lung cancer is assessed for expression of immune-mediated biomarkers that are related to response or clinical benefit of the anti-PD-1 antibody, such as tislelizumab, e.g., using tumor biopsy. The biomarker can be, without limitation, PD-L1, TMB, GEP and MSI. Biomarkers expression can be assessed by multiplex immunohistochemistry assay (IHC). The lung cancer to be treated can be a cancer that has a detectable expression of PD-L1. The lung cancer to be treated can be a cancer that has a detectable expression of TMB. The lung cancer to be treated can be a cancer that has a detectable expression of GEP (such as immune-mediated GEP). The lung cancer to be treated can be a cancer that has a detectable expression of MSI.

In some embodiments, the cells of the lung cancer comprise high level of PD-L1 expression as measured by the percentage of PD-L1 positive tumor or immune cells or as measured by the areas occupied by PD-L1 positive tumor and immune cells, divided by the total tumor area. In some embodiments, the subject has a low level of PD-L1 expression as measured by the percentage of PD-L1 positive tumor or immune cells or as measured by the areas occupied by PD-L1 positive tumor and immune cells, divided by the total tumor area. In some embodiments, the elevated level of PD-L1 expression is at least 1%, 2%, 5%, 10%, 20%, or 50%.

In some embodiments, the cells of the lung cancer have intermediate or high tumor mutational burden, optionally wherein the tumor has more than 5 or between 5 and 20 mutations, more than 20 or between 20 and 50 mutations, or more than 50 mutations.

In some embodiments, the cells of the lung cancer comprise an epidermal growth factor (EGFR) mutation. In some embodiments, the cells of the lung cancer comprise a v-Ki-ras2 Kirsten rat sarcoma viral oncogene homolog (KRAS) mutation. In some embodiments, the cells of the lung cancer comprise an ALK mutation.

In some embodiments, the cells of the lung cancer are EGFR genomic aberration negative. In some embodiments, the cells of the lung cancer are KRAS genomic aberration negative. In some embodiments, the cells of the lung cancer are ALK genomic aberration negative.

In some embodiments, the SCLC is characterized by expression of one or more of INSM1, CD56/NCAM, synptophysin, or chromogranin A. In some embodiments, the SCLC is characterized by expression of INSM1. In some embodiments, the SCLC is characterized by expression of CD56/NCAM. In some embodiments, the SCLC is characterized by expression of synptophysin. In some embodiments, the SCLC is characterized by expression of chromogranin.

In some embodiments, the SCLC is characterized by more than 16.3 ng/ml of neuron-specific enolase (NSE). In some embodiments, the SCLC is characterized by more than 66 μg/ml progastrin-releasing peptide (ProGRP). In some embodiments, the SCLC is characterized by more than 2.5 ng/ml carcinoembryonic antigen (CEA). In some embodiments, the SCLC is characterized by more than 10 ng/ml carcinoembryonic antigen (CEA). In some embodiments, the SCLC is characterized by more than 16.3 ng/ml of neuron-specific enolase (NSE), more than 66 μg/ml progastrin-releasing peptide (ProGRP), and more than 2.5 ng/ml or more than 10 ng/ml carcinoembryonic antigen (CEA).

### Patient Populations Treated as Provided Herein

In some embodiments, the patient is a mammal. In some embodiments, the mammal is human. In some embodiments, the patient is an adult. In some embodiments, the patient is 18 years of age or over 18 years of age. In some embodiments, the patient is over 40 years of age, over 45 years of age, over 50 years of age, over 55 years of age, over 60 years of age, over 65 years of age, over 70 years of age, over 75 years of age, or over 80 years of age. In some embodiments, the patient is 65 years or older.

In some embodiments, the patient is male. In some embodiments, the patient is female.

In some embodiments, the patient is a current smoker. In some embodiments, the patient is a former smoker. In some embodiments the patient has never smoked or was never a smoker.

In some embodiments, the subject has (e.g., has been diagnosed with) lung cancer. In some embodiments, the subject has any of the cancers described herein. In some embodiments, the subject has mid-stage lung cancer. In some embodiments, the subject has late-stage lung cancer. In some embodiments, the subject has (e.g., has been diagnosed with) small cell lung cancer. In some embodiments, the subject has (e.g., has been diagnosed with) extensive stage small cell lung cancer (ES-SCLC). In some embodiments, the subject has (e.g., has been diagnosed) with stage IIIA SCLC. In some embodiments, the subject has (e.g., has been diagnosed) with stage IIIB SCLC. In some embodiments, the subject has (e.g., has been diagnosed) with stage IV SCLC. In some embodiments, the subject has (e.g., has been diagnosed) small cell carcinoma. In some embodiments, the subject has (e.g., has been diagnosed) combined small cell carcinoma.

In some embodiments, the subject has (e.g., has been diagnosed with) metastatic cancer. In some embodiments, the subject has one or more of liver, lung, lymph node, bone, and/or adrenal gland metastasis. In some embodiments, the subject has liver, lung or lymph node metastasis. In some embodiments, the subject has liver metastasis. In some embodiments, the subject has lung metastasis. In some embodiments, the subject has lymph node metastasis. In some embodiments, the subject has bone metastasis. In some embodiments, the subject has adrenal gland metastasis. In some embodiments, the subject has 1, 2, 3, or more metastatic sites. In some embodiments, the subject has 1 or more metastatic sites. In some embodiments, the subject has 2 or more metastatic sites. In some embodiments, the subject has 3 or more metastatic sites. In some embodiments, the subject does not have brain metastasis. In other embodiments, the subject has brain metastasis.

In some embodiments, the subject has been diagnosed using histological markers. In some embodiments, the subject has been diagnosed using cytological markers. In some embodiments, the subject has been diagnosed using histological and cytological markers. In some embodiments, the subject has been diagnosed using a biopsy. In some embodiments, the subject has been diagnosed using a solid biopsy. In some embodiments, the subject has been diagnosed with a liquid biopsy. Solid tissue biopsy involves sampling a piece of tissue from a node or tumor for examination. Liquid biopsy is a minimally invasive approach for sample procurement, mostly body fluids, and it is used to detect molecular alterations in shed, circulating tumor cells.

In some embodiments, the subject has not previously been treated with an immunotherapy. In some embodiments, the subject has not previously been treated with a chemotherapy (e.g., platinum-based chemotherapy). In some embodiments, the subject has not previously been treated with any immune checkpoint inhibitor. In some embodiments, the subject has not previously been treated with an immunotherapy or a chemotherapy. In some embodiments, the subject has not previously been treated with an immunotherapy and/or a chemotherapy for the cancer being treated in accordance with the methods described herein. In some embodiments, the subject has received prior chemotherapy for limited stage SCLC but has not been treated for extensive stage SCLC. In some of the embodiments where the subject has been treated for limited stage SCLC, there is a treatment-free interval of 6 months or more before the diagnosis of ES-SCLC and/or the initiation of the treatment described herein.

In some embodiments, the subject has received prior chemotherapy treatment (e.g., for the cancer being treated). In some embodiments, the subject has received prior immunotherapy treatment (e.g., for the cancer being treated).

In some embodiments, the subject has adequate organ function, e.g., as measured by one or more of the criteria described herein.

In some embodiments, the subject has no renal dysfunction. In some embodiments, the subject does not have sever renal dysfunction. In some embodiments, the subject has a moderate renal dysfunction as described herein. In some embodiments, the subject has glomerular filtration rate of more than 30 mL/min/1.73 m² and less than 60 mL/min/1.73 m² by Chronic Kidney Disease Epidemiology Collaboration equation.

### Uses and Methods of Treatment Provided Herein

In some embodiments, the disclosure relates to a method of treating lung cancer (such as SCLC) in a subject (e.g., a human), the method comprising administering to the subject effective amounts of an anti-PD-1 antibody and one or more chemotherapeutic drug (e.g., a platinum chemotherapy drug). In some embodiments, the one or more chemotherapeutic drugs is cisplatin or carboplatin. In some embodiments, the one or more chemotherapeutic drugs is etoposide.

In some embodiment, the disclosure relates to a method of treating lung cancer (such as SCLC) in a subject (e.g., a human), the method comprising administering to the subject an effective amounts of an anti-PD-1 antibody, cisplatin or carboplatin, and etoposide. In some embodiments, the subject is treated in accordance with a treatment regimen (such as dosing, frequency and duration of the treatment) described herein.

In some embodiments, the treating is clinically effective or the subject is a responder to the treating by at least one measure of response to treatment. In some embodiments, the response to treatment is measured by overall survival, progression-free or event-free survival, overall response or objective response rate, complete response, partial response, and/or duration of response. In some embodiments, the response to treatment is measured by tumor volume. In some embodiments, the treating reduces tumor volume by at least 5%, at least 10%, at least 15%, at least 20%, at least 25%, at least 30%, at least 35%, at least 40%, at least 45%, at least 50%, or at least 75%. In some embodiments, the subject has greater than 30% probability of survival at 24 months. In some embodiments, the treating is effective as demonstrated by patient population overall survival rate at 24 months of greater than 30%. In some embodiments, the subject has greater than 15% or 20% probability of event-free survival at 12 months. In some embodiments, the treating is effective as demonstrated by patient population event-free survival rate at 12 months of greater than 20%.

The anti-PD-1 antibody and one or more chemotherapeutic drugs disclosed herein may be administered parenterally. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. In some embodiments, the anti-PD-1 antibody and chemotherapeutic drug(s) may be administered by different routes. In some embodiments, the anti-PD-1 antibody is administered intravenously, and the one or more chemotherapeutic drugs are also administered intravenously.

In some embodiments, described herein is a method of treating lung cancer (e.g., SCLC or ES-SCLC) in a subject in need thereof, comprising administering to the subject a therapeutically effective amount of an anti-PD-1 antibody, wherein the PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36, and a therapeutically effective amount of one or more chemotherapeutic drugs. In some embodiments, described herein is a method of treating lung cancer (e.g., SCLC or ES-SCLC) in a subject in need thereof, comprising administering to the subject a therapeutically effective amount of an anti-PD-1 antibody, wherein the PD-1 antibody comprises a heavy chain variable region (Vh) and a light chain variable region (Vl) comprising SEQ ID NO: 24 and SEQ ID NO: 26, respectively, and a therapeutically effective amount of one or more chemotherapeutic drugs. In some embodiments, described herein is a method of treating lung cancer (e.g., SCLC or ES-SCLC) in a subject in need thereof, comprising administering to the subject a therapeutically effective amount of an anti-PD-1 antibody, wherein the PD-1 antibody is a monoclonal antibody or a fragment thereof (e.g., a humanized antibody or fragment), comprising a heavy chain variable region (Vh) amino acid sequence of SEQ ID NO: 24, a light chain variable region (Vl) amino acid sequence of SEQ ID NO: 26, and an IgG4 constant domain amino acid sequence of SEQ ID NO: 88. In some embodiments, described herein is a method of treating lung cancer (e.g., SCLC or ES-SCLC) in a subject in need thereof, comprising administering to the subject a therapeutically effective amount of Tislelizumab and a therapeutically effective amount of one or more chemotherapeutic drugs.

In some embodiments, a subject is administered one or more, or all of the study drugs provided in Table 8, at the treatment regimen described therein. In some embodiments, the choice between cisplatin and carboplatin is at treating physician's discretion.

TABLE 8
Selection and Timing of Dose for Patients ab

| | | Frequency of | Route of | |
|---|---|---|---|---|
| Study Drug | Dose | Administration | Administration | Duration of Treatment |
| Tislelizumab | 200 mg | Day 1 of every | IV | 4 cycles of induction |
| | | 3 weeks | | therapy, followed by |
| | | | | maintenance treatment |
| | | | | (cycle 5 and beyond) |
| Cisplatin | 75 mg/m² | Day 1 of every | IV | 4 cycles of induction |
| | | 3 weeks | | therapy only |
| Carboplatin | AUC of 5 | Day 1 of every | IV | |
| | mg/mL/min | 3 weeks | | |
| Etoposide | 100 mg/m² | Days 1, 2 and 3 | IV | 4 cycles of induction |
| | | every 3 weeks | | therapy only |

In some embodiments, a subject is administered Tislelizumab in combination with Cisplatin and Etoposide as described in Table 2. In some embodiments, a subject is administered Tislelizumab in combination with Carboplatin and Etoposide as described in Table 3.

In some embodiments, the disclosure provides a method of treating small cell lung cancer (e.g., ES-SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) administering an anti-PD-1 antibody, (ii) administering Cisplatin or Carboplatin, and (iii) administering Etoposide; and (b) the Maintenance Period comprises (i) administering an anti-PD-1 antibody.

In some embodiments, the disclosure provides a method of treating small cell lung cancer (e.g., SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) at least one administration cycle, wherein each cycle is three weeks, and (ii) administering an anti-PD-1 antibody, (iii) administering Cisplatin or Carboplatin, and (iv) administering Etoposide; and (b) the Maintenance Period comprises: (i) at least one administration cycle, wherein each cycle is three weeks, and (ii) administering an anti-PD-1 antibody.

In some embodiments, the disclosure provides a method of treating small cell lung cancer (e.g., SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) four administration cycles, wherein each cycle is three weeks, and (ii) administering an anti-PD-1 antibody, (iii) administering Cisplatin or Carboplatin, and (iv) administering Etoposide; and (b) the Maintenance Period comprises: (i) at least one administration cycle, wherein each cycle is three weeks, and (ii) administering an anti-PD-1 antibody.

In some embodiments, the disclosure provides a method of treating small cell lung cancer (e.g., SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) four administration cycles, wherein each cycle is three weeks, (ii) administering an anti-PD-1 antibody on Day 1 of each cycle, (iii) administering Cisplatin or Carboplatin, on Day 1 of each cycle, and (iv) administering Etoposide on Day 1, Day 2, and Day 3 of each cycle; and (b) the Maintenance Period comprises: (i) at least one administration cycle, wherein each cycle is three weeks, and (ii) administering an anti-PD-1 antibody on Day 1 of each cycle.

In some embodiments, the disclosure provides a method of treating small cell lung cancer (e.g., SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) four administration cycles, wherein each cycle is three weeks, (ii) administering an anti-PD-1 antibody at 200 mg on Day 1 of each cycle, (iii) administering Cisplatin at 75 mg/m² or Carboplatin at AUC 5, on Day 1 of each cycle, and (iv) administering Etoposide at 100 mg/m² on Day 1, Day 2, and Day 3 of each cycle; and (b) the Maintenance Period comprises (i) at least one administration cycle, wherein each cycle is three weeks, and (ii) administering an anti-PD-1 antibody at 200 mg on Day 1 of each cycle.

In some embodiments, the disclosure provides a method of treating small cell lung cancer (e.g., SCLC) in a human subject in need thereof, comprising administering to the subject a therapeutically effective amount of an anti-PD-1 antibody or an antigen-binding fragment thereof (e.g., tislelizumab or an antibody or fragment having the CDRs thereof), and a therapeutically effective amount of one or more chemotherapeutic drugs, wherein the method comprises an Induction Period and a Maintenance Period, wherein: (a) the Induction Period comprises: (i) four administration cycles, wherein each cycle is three weeks, (ii) administering an anti-PD-1 antibody at 200 mg on Day 1 of each cycle, wherein the anti-PD-1 antibody is infused over 60 minutes during the first cycle and is infused over 30 minutes in the second, third, and fourth cycles, (iii) waiting at least 60 minutes after infusion of the anti-PD-1 antibody prior to administration of the chemotherapeutic drugs in the first and second cycle, and waiting at least 30 minutes after infusion of the anti-PD-1 antibody prior to administration of the chemotherapeutic drugs in the third, and fourth cycles, (iv) administering Cisplatin at 75 mg/m² or Carboplatin at AUC 5, on Day 1 of each cycle, wherein Cisplatin is infused over two hours and Carboplatin is infused over 15-60 minutes, and (v) administering Etoposide at 100 mg/m² on Day 1, Day 2, and Day 3 of each cycle, wherein Etoposide is infused over 60 minutes; and (b) the Maintenance Period comprises: (i) at least one administration cycle, wherein each cycle is three weeks, and (ii) administering an anti-PD-1 antibody at 200 mg on Day 1 of each cycle, wherein the anti-PD-1 antibody is infused over 30 minutes.

In some embodiments, the maintenance period comprises administration of an anti-PD-1 antibody but does not comprise administration of a chemotherapeutic drug (e.g., cisplatin, carboplatin and/or etoposide).

Embodiments of the present disclosure also include the agents and combinations described herein for use in, for use as a medicament or composition for, or for use in the preparation of a medicament for any one or more of:
a. therapy (e.g., of the human body);
b. medicine;
c. induction of or augmenting an anti-tumor immune response;
d. decreasing the number of one or more tumor markers in a patient;
e. halting or delaying the growth of the cancer described herein;
f. halting or delaying the progression of the cancer described herein;
g. halting or delaying the progression of the cancer described herein;
h. stabilization of the cancer;
i. inhibiting the growth or survival of the cancer cells;
j. eliminating or reducing the size of one or more cancerous lesions;
k. reduction of the progression, onset or severity of the cancer described herein;
l. reducing the severity or duration of the clinical symptoms of the cancer described herein;
m. prolonging the survival of a patient relative to the expected survival in a similar untreated patient;
n. inducing complete or partial remission of the cancer described herein; or
o. treatment of the cancer described herein.

### Response to Treatment

In some embodiments, the patient is responsive to treatment by one, two, three or more criteria described herein and/or known in the art. In some embodiments, the responsiveness criteria are any criterial described herein, including but not limited to those described in the example section of this application.

In some embodiments, the treatment described herein reduces tumor volume, size, or diameter. In some embodiments, the treatment described herein reduces tumor volume, size or diameter by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the treatment described herein reduces the diameter of a cancer lesion by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the measurements are made by CT or MRI. In some embodiments, the measurements are made and results are assessed as described herein (see, e.g., the example section). In some embodiments, the reduction is relative to the tumor volume, size or diameter in the subject prior to the start of the treatment.

In some embodiments, the methods described herein improve overall survival. In some embodiments, the improvement is relative to probability of survival without treatment or with a standard of care treatment.

In some embodiments, the response to treatment in a subject is measured by overall survival, progression-free survival, event-free survival, overall response rate, objective response rate, partial response, duration of response, or a combination thereof. In some embodiments, the response to treatment in a subject is measured by overall survival. In some embodiments, the response to treatment in a subject is measured by progression-free survival. In some embodiments, the response to treatment in a subject is measured by event-free survival. In some embodiments, the response to treatment in a subject is measured by overall response rate. In some embodiments, the response to treatment in a subject is measured by objective response rate. In some embodiments, the response to treatment in a subject is measured by partial response. In some embodiments, the response to treatment in a subject is measured by duration of response.

In some embodiments, the patient survives for at least 24 months. In some embodiments, the patient survives for at least 27 months. In some embodiments, the patient survives for at least 30 months. In some embodiments, the patient survives for at least 36 months. In some embodiments, the patient survives for at least 39 months. In some embodiments, the patient survives for at least 42 months. In some embodiments, the patient survives for at least 45 months. In some embodiments, the patient survives for at least 48 months or at least 4 years. In some embodiments, the patient survives for at least 5 years. In some embodiments, the survival determination is starting from the initiation of treatment (e.g., the specified months since the treatment initiation).

In some embodiments, the subject has a greater than 30% probability of survival at 24 months from initiation of a treatment regimen described herein.

In some embodiments, the treating prolongs survival of the human patient which prolonged survival can be free of progression of the lung cancer. In some embodiments, the treating prolongs survival of the human patient which prolonged survival can be free of progression of the small cell lung cancer. In some embodiments, the subject has a greater than 15% probability of event-free survival at 12 months from initiation of a treatment regimen described herein. In some embodiments, the subject has a greater than 20% probability of event-free survival at 12 months from initiation of a treatment regimen described herein.

In some embodiments, the patient survives free of progression of the SCLC for at least 4 months. In some embodiments, the patient survives free of progression of the SCLC for at least 5 months. In some embodiments, the patient survives free of progression of the SCLC for at least 6 months. In some embodiments, the patient survives free of progression of the SCLC for at least 7 months. In some embodiments, the patient survives free of progression of the SCLC for at least 8 months. In some embodiments, the patient survives free of progression of the SCLC for at least 9 months. In some embodiments, the patient survives free of progression of the SCLC for at least 10 months. In some embodiments, the patient survives free of progression of the SCLC for at least 11 months. In some embodiments, the patient survives free of progression of the SCLC for at least 12 months. In some embodiments, the patient survives free of progression of the SCLC for at least 15 months. In some embodiments, the patient survives free of progression of the SCLC for at least 18 months. In some embodiments, the patient survives free of progression of the SCLC for at least 21 months. In some embodiments, the patient survives free of progression of the SCLC for at least 24 months. In some embodiments, the patient survives free of progression of the SCLC for at least 30 months. In some embodiments, the patient survives free of progression of the SCLC for at least 34 months. In some embodiments, the patient survives free of progression of the SCLC for at least 35 months. In some embodiments, the patient survives free of progression of the SCLC for at least 36 months or 3 years. In some embodiments, the patient survives free of progression of the SCLC for at least 39 months. In some embodiments, the patient survives free of progression of the SCLC for at least 42 months. In some embodiments, the patient survives free of progression of the SCLC for at least 45 months. In some embodiments, the patient survives free of progression of the SCLC for at least 48 months or 4 years. In some embodiments, the patient survives free of progression of the SCLC for at least 5 years. In some embodiments, the survival free of progression determination is starting from the initiation of treatment (e.g., the specified months since the treatment initiation).

In some embodiments, the patient exhibits the duration of response of at least 6 months. In some embodiments, the patient exhibits the duration of response of at least 12 months. In some embodiments, the patient exhibits the duration of response of at least 18 months. In some embodiments, the patient exhibits the duration of response of at least 24 months. In some embodiments, the patient exhibits the duration of response of at least 30 months. In some embodiments, the patient exhibits the duration of response of at least 33 months. In some embodiments, the patient exhibits the duration of response of at least 36 months. In some embodiments, the patient exhibits the duration of response of at least 39 months. In some embodiments, the patient exhibits the duration of response of at least 42 months. In some embodiments, the patient exhibits the duration of response of at least 45 months. In some embodiments, the patient exhibits the duration of response of at least 48 months or 4 years. In some embodiments, the patient exhibits the duration of response of at least 5 years. In some embodiments, the responsiveness determination is starting from the initiation of treatment (e.g., the specified months since the treatment initiation).

In some embodiments, the response to treatment in a subject is measured by overall response rate, complete response and/or partial response. In some embodiments, the determination of these responsiveness endpoints are determined using RECIST v1.1.

In some embodiments, a human responder to treatment exhibits stable disease. In some embodiments, a human responder to treatment exhibits stable disease for, or at least for, 3 months, 6 months, 9 months, 12 months, 18 months, 21 months, 24 months, 30 months, 36 months, 42 months, 48 months or 5 years. In some embodiments, the determination is starting from the initiation of treatment (e.g., the specified months since the treatment initiation).

In some embodiments, a human responder to treatment does not exhibit progressive disease, and/or exhibits delay in the cancer progression. In some embodiments, a human responder to treatment does not exhibit progressive disease, and/or exhibits delay in the cancer progression for, or at least for, 3 months, 6 months, 9 months, 12 months, 18 months, 21 months, 24 months, 30 months, 36 months, 42 months, 48 months or 5 years. In some embodiments, the determination is starting from the initiation of treatment (e.g., the specified months since the treatment initiation).

In some embodiments, a human responder to treatment exhibits a response (such as an improvement or delay of progression response) in one or more patient-reported outcome (PRO) assessments, such as any one of the PRO assessments described herein (including but not limited to Quality of Life assessments using any of the Questionnaires described herein). In some embodiments, a positive response on a PRO assessment is observed for, or at least for, 3 months, 6 months, 9 months, 12 months, 18 months, 21 months, 24 months, 30 months, 36 months, 42 months, 48 months or 5 years starting from the initiation of treatment.

In some embodiments, a human responder to treatment as described herein has been diagnosed and treated for Stage IIIA SCLC. In some embodiments, a human responder to treatment as described herein has been diagnosed and treated for Stage IIIB SCLC. In some embodiments, a human responder to treatment as described herein has been diagnosed and treated for Stage IV SCLC. In some embodiments, a human responder to treatment as described herein has been diagnosed and treated for ES-SCLC. In some embodiments, a human responder to treatment as described herein has been diagnosed and treated for metastatic SCLC (e.g., having more than 3 metastatic sites). In some embodiments, a human responder to treatment as described herein has been diagnosed and treated for extensive, unresectable and metastatic SCLC.

In some embodiments, a human responder to treatment is free of progression of Stage IIIA SCLC. In some embodiments, a human responder to treatment is free of progression of Stage III SCLC. In some embodiments, a human responder to treatment is free of progression of Stage IV SCLC. In some embodiments, a human responder to treatment is free of progression of ES-SCLC. In some embodiments, a human responder to treatment is free of progression of metastatic SCLC (e.g., having more than 3 metastatic sites). In some embodiments, a human responder to treatment is free of progression of extensive, unresectable and metastatic SCLC.

### Pharmaceutical Compositions

Provided herein are compositions, including pharmaceutical formulations, comprising an anti-PD-1 antibody or an antigen-binding fragment thereof described herein. Also provided herein are compositions, including pharmaceutical formulations, comprising one or more chemotherapeutic drugs described herein. In some embodiments, the composition, including a pharmaceutical formulation, comprises an anti-PD-1 antibody or an antigen-binding fragment thereof and one or more chemotherapeutic drugs.

Pharmaceutical formulations of an anti-PD-1 antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In some embodiments, the surfactant is a polysorbate. Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer. In some embodiments, the formulations include a histidine-citric acid buffer. Sustained-release preparations can be prepared. Suitable examples of sustained-release preparations include semipermeable matrices of solid hydrophobic polymers containing the antibody, which matrices are in the form of shaped articles, e.g., films, or microcapsules. The formulations to be used for in vivo administration are generally sterile. Sterility can be readily accomplished, e.g., by filtration through sterile filtration membranes.

In some embodiments, the pharmaceutical composition or formulation comprises a formulation buffer comprising histidine, acetate, citrate, succinate, phosphate, mixture of histidine and acetic acid, mixture of histidine and citric acid, salt or hydrate thereof, or any combination of them. In some embodiments, the formulation buffer comprises a histidine buffer that comprises histidine, histidine hydrochloride, L-histidine, L-histidine hydrochloride, L-histidine hydrochloride hydrate, L-histidine hydrochloride monohydrate, or a combination thereof. In some embodiments, the histidine buffer comprises L-histidine/L-histidine hydrochloride monohydrate buffer (His/His HCl). In some embodiments, the histidine buffer comprises histidine/L-histidine hydrochloride monohydrate buffer and citric acid monohydrate. In some embodiments, the concentration of the histidine buffer (e.g., L-histidine/L-histidine hydrochloride monohydrate) is between about 5 mM to about 30 mM. In some embodiments, the concentration of the histidine buffer (e.g., L-histidine/L-histidine hydrochloride monohydrate) is between about 5 mM to about 10 mM, between about 5 mM to about 15 mM, between about 5 mM to about 20 mM, between about 10 mM to about 15 mM, between about 10 mM to about 20 mM, between about 10 mM to about 25 mM, between about 15 mM to about 20 mM, between about 15 mM to about 25 mM, between about 15 mM to about 30 mM, between about 20 mM to about 25 mM, or between about 25 mM to about 30 mM. In some embodiments, the concentration of the histidine buffer (e.g., L-histidine/L-histidine hydrochloride monohydrate) is about 5 mM or more, about 10 mM or more, about 15 mM or more, about 20 mM or more, about 25 mM or more, about 30 mM or more. In some embodiments, the concentration of the histidine buffer (e.g., L-histidine/L-histidine hydrochloride monohydrate) is 10 mM.

In some embodiments, the concentration of the histidine is about 5-15 mM, in a preferred embodiment the concentration of histidine is about 11 mM. In some embodiments, the concentration of the L-histidine hydrochloride monohydrate is about 1-5 mM, in a preferred embodiment the concentration of L-histidine hydrochloride monohydrate is about 4 mM. In some embodiments, the formulation comprises histidine at a concentration of about 5-15 mM, in a preferred embodiment the concentration of histidine is about 11 mM, and L-histidine hydrochloride monohydrate at a concentration of about 1-5 mM, in a preferred embodiment the concentration of L-histidine hydrochloride monohydrate is about 4 mM. In some embodiments, the formulation buffer is a mixture of histidine, L-histidine hydrochloride monohydrate, citric acid monohydrate, and sodium citrate; wherein the formulation comprises histidine at a concentration of about 5-15 mM, in a preferred embodiment the concentration of histidine is about 11 mM, and L-histidine hydrochloride monohydrate at a concentration of about 1-5 mM, in a preferred embodiment the concentration of L-histidine hydrochloride monohydrate is about 4 mM, wherein the citric acid monohydrate is at a concentration of about 1-5 mM, preferably 2 mM, and sodium citrate is present at a concentration of 15-30 mM, preferably about 23 mM.

In some embodiments, the pharmaceutical composition or formulation comprises a stabilizer. In some embodiments, the stabilizer comprises trehalose, sucrose, sorbitol, mannitol, L-arginine hydrochloride, maltose, dextran, (2-hydroxypropyl)-b-cyclodextrin, sodium chloride, magnesium chloride, calcium chloride, sodium sulfate, sodium dihydrogen phosphate, disodium hydrogen phosphate, or a combination thereof. In some embodiments, the stabilizer comprises trehalose, sucrose, L-arginine hydrochloride, sodium chloride, or a combination thereof. In some embodiments, the stabilizer is trehalose. In some embodiments the trehalose is a-trehalose dihydrate. In some embodiments, the concentration of the stabilizer is between about 50 mM to about 300 mM. In some embodiments, the concentration of the stabilizer is between about 50 mM to about 100 mM, between about 50 mM to about 150 mM, between about 50 mM to about 200 mM, between about 100 mM to about 150 mM, between about 100 mM to about 200 mM, between about 100 mM to about 250 mM, between about 150 mM to about 200 mM, between about 150 mM to about 250 mM, or between about 150 mM to about 300 mM. In some embodiments, the concentration of the stabilizer is about 50 mM or more, about 60 mM or more, about 70 mM or more, about 80 mM or more, about 90 mM or more, about 100 mM or more, about 110 mM or more, about 120 mM or more, about 130 mM or more, about 140 mM or more, about 150 mM or more, about 160 mM or more, about 170 mM or more, about 180 mM or more, about 190 mM or more, about 200 mM or more, about 210 mM or more, about 220 mM or more, about 230 mM or more, about 240 mM or more, about 250 mM or more, about 260 mM or more, about 270 mM or more, about 280 mM or more, about 290 mM or more, or about 300 mM or more. In some embodiments, the concentration of the stabilizer is about 190 mM.

In some embodiments, the pharmaceutical composition or formulation comprises a non-ionic surfactant. In some embodiments, the non-ionic surfactant comprises polysorbate 80 (PS80), polysorbate 20 (PS20), poloxamer188 (P188), or a combination thereof. In some embodiments, the non-ionic surfactant is PS20. In some embodiment, the concentration of the non-ionic surfactant is between about 0.01% to about 1%. In some embodiments, the concentration of the non-ionic surfactant is between about 0.01% to about 0.03%, between about 0.01% to about 0.06%, between about 0.01% to about 0.09%, between about 0.03% to about 0.06%, between about 0.03% to about 0.09%, between about 0.03% to about 0.2%, between about 0.06% to about 0.09%, between about 0.06% to about 0.2%, between about 0.06% to about 0.5%, between about 0.09% to about 0.2%, between about 0.09% to about 0.5%, between about 0.09% to about 0.8%, between about 0.2% to about 0.5%, between about 0.2% to about 0.8%, between about 0.2% to about 1%, between about 0.5% to about 0.8%, between about 0.5% to about 1%, or between about 0.8% to about 1%. In some embodiments, the concentration of the non-ionic surfactant is about 0.01% or more, is about 0.02% or more, is about 0.03% or more, is about 0.04% or more, is about 0.05% or more, is about 0.06% or more, is about 0.07% or more, is about 0.08% or more, is about 0.09% or more, is about 0.1% or more, is about 0.2% or more, is about 0.3% or more, is about 0.4% or more, is about 0.5% or more, is about 0.6% or more, is about 0.7% or more, is about 0.8% or more, is about 0.9% or more, or is about 1% or more. In some embodiments, the concentration of the non-ionic surfactant is 0.2%.

In some embodiments, the pharmaceutical composition or formulation comprises between about 1 mg/ml to about 10 mg/ml of the antibody such as tislelizumab as disclosed herein. In some embodiments, the pharmaceutical composition or formulation comprises between about 1 mg/ml to about 2 mg/ml, between about 1 mg/ml to about 3 mg/ml, between about 1 mg/ml to about 4 mg/ml, between about 2 mg/ml to about 3 mg/ml, between about 2 mg/ml to about 4 mg/ml, between about 2 mg/ml to about 5 mg/ml, between about 3 mg/ml to about 4 mg/ml, between about 3 mg/ml to about 5 mg/ml, between about 3 mg/ml to about 6 mg/ml, between about 4 mg/ml to about 5 mg/ml, between about 4 mg/ml to about 6 mg/ml, between about 4 mg/ml to about 7 mg/ml, between about 5 mg/ml to about 6 mg/ml, between about 5 mg/ml to about 7 mg/ml, between about 5 mg/ml to about 8 mg/ml, between about 6 mg/ml to about 7 mg/ml, between about 6 mg/ml to about 8 mg/ml, between about 6 mg/ml to about 9 mg/ml, between about 7 mg/ml to about 8 mg/ml, between about 7 mg/ml to about 9 mg/ml, between about 7 mg/ml to about 10 mg/ml, between about 8 mg/ml to about 9 mg/ml, between about 8 mg/ml to about 10 mg/ml, or between about 9 mg/ml to about 10 mg/ml of the antibody disclosed herein. In some embodiments, the pharmaceutical composition or formulation comprises about 1 mg/ml or more, 2 mg/ml or more, 3 mg/ml or more, 4 mg/ml or more, 5 mg/ml or more, 6 mg/ml or more, 7 mg/ml or more, 8 mg/ml or more, 9 mg/ml or more, or 10 mg/ml or more of the antibody disclosed herein. In some embodiments, the pharmaceutical composition or formulation comprises 10 mg/ml of the antibody (e.g. PD-1 antibody or tislelizumab) disclosed herein.

In some embodiments, the pharmaceutical composition or formulation has a pH of between about 4.5 to about 7.5. In some embodiments, the pharmaceutical composition or formulation has a pH of between about 4.5 to about 5.0, between about 4.5 to about 5.5, between about 4.5 to about 6.0, between about 5.0 to about 5.5, between about 5.0 to about 6.0, between about 5.0 to about 6.5, between about 5.5 to about 6.0, between about 5.5 to about 6.5, between about 5.5 to about 7.0, between about 6.0 to about 6.5, between about 6.0 to about 7.0, between about 6.0 to about 7.5, between about 6.5 to about 7.0, between about 6.5 to about 7.5, or between about 7.0 to about 7.5. In some embodiments, the pharmaceutical composition or formulation has a pH of about 4.5 or more, about 5.0 or more, about 5.5 or more, about 6.0 or more, about 6.5 or more, about 7.0 or more, or about 7.5 or more. In some embodiments, the pharmaceutical composition or formulation has a pH of 6.5.

In some embodiments, the antibody (e.g. PD-1 antibody or tislelizumab) is formulated in a pharmaceutical composition comprising a histidine buffer, a surfactant and a stabilizer. In some embodiments, the surfactant is a polysorbate. In some embodiments, the stabilizer is trehalose. In a particular embodiment, the PD-1 antibody (e.g. tislelizumab) is formulated in a pharmaceutical composition comprising citric acid monohydrate, histidine, L-histidine hydrochloride monohydrate, polysorbate 20, sodium citrate, and trehalose.

The term “pharmaceutical composition” refers to preparations with pharmaceutically acceptable excipients which are in such form as to permit the active ingredients to be effective, and which contains no additional components which are toxic to the subjects to which the composition would be administered. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some aspects, the present disclosure provides compositions, e.g., pharmaceutically acceptable compositions, which include an anti-PD-1 antibody described herein, formulated together with at least one pharmaceutically acceptable excipient.

The term “pharmaceutically acceptable” refers to a molecule or composition that, when administered to a recipient, is not deleterious to the recipient thereof, or that any deleterious effect is outweighed by a benefit to the recipient thereof.

Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

A “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g., by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. Typical suitable compositions are in the form of injectable or infusion solutions. One suitable mode of administration is parenteral (e.g., intravenous, subcutaneous, intraperitoneal, intramuscular). In some embodiments, the antibody is administered by intravenous infusion or injection. In some embodiments, the antibody is administered by intramuscular or subcutaneous injection. In some embodiments, the antibody is administered by way of a syringe infusion system.

### Kits

The present disclosure, among other things, provides kits comprising anti-PD-1 antibodies or antigen-binding fragments thereof, and instructions for use and/or administration. In some embodiments, a kit comprises at least one anti-PD-1 antibody or antigen-binding fragment thereof and a pharmaceutically acceptable carrier, and instructions for use and/or administration. In some embodiments, a kit comprises at least one anti-PD-1 antibody or antigen-binding fragment thereof, at least one chemotherapeutic agent, and a pharmaceutically acceptable carrier, and instructions for use and/or administration.

Also provided are kits for use in various methods disclosed herein. Instructions can comprise a description of administering of one or more pharmaceutical compositions described herein to a subject to achieve an intended activity in a subject. A kit may further comprise a description of selecting a human suitable for treatment based on identifying whether the human is in need of treatment. In some embodiments, instructions comprise a description of administering at least one anti-PD-1 antibody or antigen-binding fragment thereof to a subject who is in need of the treatment.

Instructions relating to administering one or more doses of at least one anti-PD-1 antibody or antigen-binding fragment thereof, and/or one or more chemotherapeutic drug, generally include information as to dosage, dosing schedule, and route of administration for an intended treatment. Containers may be unit doses, bulk packages (e.g., multi-dose packages) or sub-unit doses. Instructions supplied in kits of the disclosure are typically written instructions on a label or package insert. A label or package insert may indicate that one or more pharmaceutical compositions described herein are used for treating, delaying the onset, and/or alleviating a disease, disorder or condition in a subject.

In some embodiments, kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, a container may be an intravenous solution bag or a vial having a stopper pierce able by a hypodermic injection needle). A container may also have a sterile access port. Kits can include additional components such as buffers and interpretive information. A kit can comprise a container and a label or one or more package inserts on or associated with a container. In some embodiment, the disclosure provides articles of manufacture comprising contents of kits described above.

Embodiments described herein are further illustrated by, though in no way limited to, the following examples.

### EXAMPLES

### Example 1. Clinical Trial in Human Patients with Extensive-Stage SCLC

This Example describes a randomized, double-blind, placebo-controlled, multicenter, Phase 3 study that was conducted, comparing the efficacy of tislelizumab (i.e., an anti-PD-1 antibody)+cisplatin or carboplatin+etoposide (Arm A) and placebo+cisplatin or carboplatin+etoposide (Arm B) as first-line treatment in 457 patients with previously untreated ES-SCLC (extensive stage-small cell lung carcinoma).

### Study Objectives

Primary Objective: To evaluate the efficacy of tislelizumab+cisplatin or carboplatin+etoposide compared with placebo+cisplatin or carboplatin+etoposide in the Intent-to-Treat (ITT) Analysis Set as measured by overall survival (OS). The choice of platinum (cisplatin or carboplatin) was at the physician's discretion.

Secondary Objectives: (1) To evaluate the efficacy of tislelizumab+cisplatin or carboplatin+etoposide compared with placebo+cisplatin or carboplatin+etoposide in the ITT Analysis Set as measured by progression-free survival (PFS) according to RECIST v1.1. (2) To evaluate the efficacy of tislelizumab+cisplatin or carboplatin+etoposide compared with placebo+cisplatin or carboplatin+etoposide in the ITT Analysis Set as measured by overall response rate (ORR), duration of response (DOR), and disease control rate (DCR) according to RECIST v1.1. (3) To evaluate the effect of tislelizumab+cisplatin or carboplatin+etoposide compared with placebo+cisplatin or carboplatin+etoposide on patients' health-related quality of life (HRQoL) according to the EORTC Quality of Life Questionnaire-Core 30 (QLQ-C30) and the supplemental lung cancer module (QLQ-LC13). (4) To evaluate the safety and tolerability of tislelizumab in combination with cisplatin or carboplatin and etoposide compared with cisplatin or carboplatin and etoposide.

Other Objectives: (1) To assess PFS after next line of treatment (PFS2). (2) To explore potential predictive biomarkers in archival and/or fresh tumor tissue and/or blood (or blood derivatives), including but not limited to PD-L1 expression by immunohistochemistry (IHC), multiplex IHC (mIHC), gene expression profiling (GEP), tumor mutation burden (TMB), microsatellite instability (MSI), and blood tumor mutation burden (bTMB), and to evaluate the association between these biomarkers and response to study treatment or mechanism of resistance. (3) To characterize PK of tislelizumab in patients with small cell lung cancer (SCLC). (4) To evaluate host immunogenicity to tislelizumab by assessing antidrug antibodies (ADAs) against tislelizumab in patients with SCLC. (5) To examine the patients' quality of life (QoL) as measured by EQ5D-5L.

### BRIEF DESCRIPTION OF METHODS

Eligible patients with previously untreated ES-SCLC and with no untreated brain metastases were randomized 1:1 to receive tislelizumab 200 mg or placebo intravenously every 3 weeks until disease progression, loss of clinical benefit, unacceptable toxicity, or withdrawal of consent. All randomized patients received concomitant induction chemotherapy (four cycles). The primary endpoint was overall survival (OS) in the intent-to-treat analysis set. Key secondary endpoints included progression-free survival (PFS), objective response rate (ORR), and duration of response (DoR) per Response Evaluation Criteria in Solid Tumors version 1.1, and safety outcomes.

### Summary of Study Design

The study consisted of a Screening phase, a Treatment phase that included an Induction treatment period and a Maintenance treatment period, a Safety Follow-up phase, and a Survival Follow-up phase.

Eligible patients (Table 11 and Table 12) were stratified by ECOG (Eastern Cooperative Oncology Group) Performance Status (0 versus 1, wherein 0 is fully active and able to carry on all pre-disease performance without restriction; 1 is restricted in physically strenuous activity but ambulatory and able to carry out work of a light or sedentary nature), investigator-chosen chemotherapy (carboplatin versus cisplatin), and brain metastasis (yes versus no), and randomized in a 1:1 ratio to receive 1 of the following treatment regimens:
Induction period; Table 9 and Table 10 (administered on a 3-week cycle for 4 cycles):
   Arm A: tislelizumab+cisplatin or carboplatin+etoposide
   Arm B: placebo+cisplatin or carboplatin+etoposide
Maintenance period (administered once every 3 weeks):
   Arm A: tislelizumab
   Arm B: placebo

### Product, Dose, and Mode of Administration:

Each of the therapeutics were administered as follows (Table 9).

Tislelizumab and placebo should be administered at a dose of 200 mg intravenously once every 3 weeks. Placebo is a sterile, preservative-free solution for infusion formulated in the same buffer as tislelizumab. All excipients used for the manufacture of placebo were of pharmacopeial grade.

Cisplatin 75 mg/m² should be administered as an intravenous infusion over 2 hours once every 3 week on Day 1 of each cycle for 4 cycles (any cisplatin-related decrease in creatinine clearance to <60 mL/min (using the Cockcroft Gault formula) requires discontinuation of cisplatin or change to carboplatin), or

Carboplatin area under the plasma or serum concentration-time curve 5 (AUC 5) should be administrated as an intravenous infusion over 15 to 60 minutes once every 3 weeks on Day 1 of each cycle for 4 cycles

Etoposide (100 mg/m²) should be administered as an intravenous infusion over 60 minutes on Days 1, 2, and 3 of each 3-week cycle for 4 cycles

The initial infusion (Cycle 1 Day 1) was delivered over 60 minutes; if this is well tolerated, then the subsequent infusions may be administered over 30 minutes, which is the shortest time period permissible for infusion. Tislelizumab or placebo must not be concurrently administered with any other drug.

A summary of the final patient populations and treatment groups is provided in Table 13. Induction treatment was administered on 21-day cycle for 4 cycles. The selection of platinum (cisplatin or carboplatin) was at the investigator's discretion and documented prior to randomization. Cycles in which no chemotherapy is given did not count toward the total number of Induction chemotherapy cycles. Following Induction treatment, patients entered a Maintenance treatment period to receive either tislelizumab or placebo alone in accordance to the treatment arm to which they were randomized. During the Maintenance treatment period prophylactic cranial irradiation (PCI) was permitted as per local standard of care. Treatment was permitted to continue until the completion of the scheduled 4 cycles (only for Induction treatment period), disease progression per RECIST v1.1, loss of clinical benefit, unacceptable toxicity, or withdrawal of informed consent, whichever occurred first.

Regular safety monitoring (at least every 6 months) and efficacy monitoring (e.g., interim analysis for overall survival) were performed by an Independent Data Monitoring Committee (IDMC).

Patients underwent tumor assessments at baseline and every 6 weeks (±7 days) for the first 48 weeks following Cycle 1 Day 1, and every 9 weeks (±7 days) thereafter, regardless of treatment dose delay. Tumor assessment at baseline must was performed within 28 days prior to randomization.

Patients were evaluated for adverse events (AEs) and immune-mediated adverse events (imAEs) (all grades according to NCI-CTCAE v5.0). Serious AEs (SAEs) and any AE were followed and documented until the event resolved, was assessed as stable, or the patient was lost to follow-up, whichever occurred first.

Primary Endpoints: Overall survival (OS), defined as the time from randomization to death from any cause. The choice of platinum (cisplatin or carboplatin) was at the physician's discretion. The primary endpoint of the study was measured by overall survival in the intention-to-treat (ITT) Analysis Set.

Secondary Endpoints: PFS, defined as the time from randomization to the first occurrence of disease progression as determined using RECIST v1.1 or death from any cause, whichever occurs first:
1. ORR, defined as the proportion of patients with PR or CR as determined using RECIST v1.1.
2. DOR, defined as the time from the first occurrence of a documented objective response to the time of relapse, as determined per RECIST v1.1, or death from any cause, whichever comes first.
3. DCR, defined as the proportion of patients whose best overall response (BOR) is CR, PR or stable disease per RECIST v1.1.
4. Incidence and severity of treatment-emergent adverse events (TEAEs) graded according to National Cancer Institute-Common Terminology Criteria for Adverse Events. (NCI-CTCAE) v5.0.
5. Percentage of patients with clinically meaningful changes post baseline, defined as a 10-point improvement or worsening (to be defined in the Statistical Analysis Plan [SAP] if otherwise) in: (1) Global health status (GHS) and physical function (PF) per the QLQ-C30, (2) Dyspnoea, coughing, haemoptysis, dysphagia, chest pain, pain in arms and shoulders, and peripheral neuropathy symptoms of QLQ-LC13
6. Time to deterioration, defined as the time from randomization to the first occurrence of worsening scores (10-point change; to be defined in the SAP if otherwise) confirmed at the following visit or death from any cause, in: (1) GHS and PF per the QLQ-C30, (2) Dyspnoea, coughing, haemoptysis, dysphagia, chest pain, pain in arms and shoulders, and peripheral neuropathy symptoms of QLQ-LC13.

Other Endpoints: (1) PFS2, defined as the time from randomization to the objective disease progression after next line of treatment or death from any cause, whichever occurs first. (2) Status of predictive and prognostic biomarkers including but not limited to PD-L1 expression, multiplex immunohistochemistry (mIHC), GEP, TMB, and MSI in archival and/or fresh tumor tissue and bTMB in blood before study treatment and/or at disease progression, and the association between the biomarkers and disease status or response to study treatment.

Summary of serum concentrations of tislelizumab. Assessments of immunogenicity of tislelizumab by determining the incidence of ADAs. QoL is defined as changes in patients' general well-being measured by the scores of the EQ5D-5L descriptive 5-dimension scores and the visual analog scale (VAS).

The following inclusion criteria were considered for patient eligibility: (1) Age ≥18 years on the day of signing the informed consent form (or the legal age of consent in the jurisdiction in which the study is taking place). (2) ECOG Performance Status of 0 or 1 (less or equal 1). (3) Histologically or cytologically confirmed ES-SCLC (defined by the American Joint Committee on Cancer, Seventh Edition, as Stage IV [T any, N any, M 1a/b] or T3-4 due to multiple lung nodules that are too extensive or have tumor/nodal volume that is too large to be encompassed in a tolerable radiation plan.) (4) Life expectancy ≥12 weeks. (5) No prior systemic treatment for ES-SCLC. Patients who have received prior chemoradiotherapy for limited-stage SCLC must have been treated with curative intent and experienced a treatment-free interval of ≥6 months between the completion of chemotherapy, radiotherapy, or chemoradiotherapy and diagnosis of ES-SCLC. (6) Adequate hematologic and end organ function as indicated by laboratory values obtained ≤7 days before randomization. (7) Absolute neutrophil count (ANC) ≥1.5×109/L, platelets ≥100×109/L, hemoglobin ≥90 g/L. Note: Patients must not have undergone a blood transfusion or growth factor support ≤14 days before sample collection at screening. (9) International normalized ratio (INR) or prothrombin time (PT)≤1.5× upper limit of normal (ULN). (10) Activated partial thromboplastin time (aPTT) ≤1.5×ULN. (11) Serum total bilirubin ≤1.5×ULN (total bilirubin has been ≤3×ULN for patients with Gilberts syndrome). (12) AST and ALT ≤2.5×ULN or AST and ALT ≤5×ULN for patients with liver metastases.

The following exclusion criteria prevented patient enrollment: (1) Active leptomeningeal disease or uncontrolled, untreated brain metastasis: (i) Patients with a history of treated and, at the time of screening, asymptomatic central nervous system (CNS) metastases were eligible if they meet all the following: (a) Brain imaging at screening shows no evidence of interim progression between the completion of CNS-directed therapy and randomization. (b) Only supratentorial metastases allowed. (c) No ongoing requirement for corticosteroids as therapy for CNS disease; anticonvulsants at a stable dose allowed. (d) No stereotactic radiation or whole-brain radiation within 14 days prior to randomization. (e) Patients with new asymptomatic CNS metastases detected at the screening scan must receive radiation therapy and/or surgery for CNS metastases. (f) Following treatment, these patients may then be eligible, provided all other criteria, including those for patients with a history of brain metastases, were met. (2) Prior therapy with an antibody or drug against immune checkpoint pathways, including but not limited to, anti-programmed cell death protein-1 (anti-PD-1), anti-PD-L1, or anti-cytotoxic T-lymphocyte-associated antigen 4 (anti-CTLA-4) antibody. (3) Was administered a live vaccine ≤4 weeks before randomization. Note: Seasonal vaccines for influenza are generally inactivated vaccines and were allowed. Intranasal vaccines are live vaccines, and were not allowed. (4) Any condition that required systemic treatment with either corticosteroids (>10 mg daily of prednisone or equivalent) or other immunosuppressive medication ≤14 days before randomization. (5) Active autoimmune diseases or history of autoimmune diseases that may relapse. Note: Patients with the following diseases were not excluded: Controlled type 1 diabetes, Hypothyroidism (provided it is managed with hormone replacement therapy only), Controlled celiac disease, Skin diseases not requiring systemic treatment (e.g., vitiligo, psoriasis, alopecia), and Any other disease that is not expected to recur in the absence of external triggering factors. (6) With a history of interstitial lung disease, non-infectious pneumonitis, or uncontrolled systemic diseases, including diabetes, hypertension, pulmonary fibrosis, acute lung diseases, etc. (7) Severe chronic or active infections requiring systemic antibacterial, antifungal or antiviral therapy within 2 weeks prior to randomization, including but not limited to tuberculosis infection. (8) Received therapeutic oral or intravenous antibiotics within 2 weeks prior to randomization. (9) Received prior therapy with an antibody or drug against immune checkpoint pathways including but not limited to anti-PD-1, anti-PD-L1, or anti-CTLA-4 antibody. (10) Treatment with systemic immune-stimulating agents (including but not limited to interferons, interleukin-2, and tumor necrosis factor) within 4 weeks or 5 half-lives of the drug, whichever is shorter, prior to randomization (prior treatment with cancer vaccines is allowed). (11) Has received any herbal medicine used to control cancer within 14 days of the first study drug administration. (12) Any condition that requires systemic treatment with either corticosteroids (>10 mg daily of prednisone or equivalent) or other immunosuppressive medication ≤14 days before randomization. Note: Patients who were currently or have previously on any of the following steroid regimens were not excluded: (i) Adrenal replacement steroid (dose ≤10 mg daily of prednisone or equivalent), (ii) Topical, ocular, intra-articular, intranasal, or inhaled corticosteroid with minimal systemic absorption, (iii) Short course (≤7 days) of corticosteroid prescribed prophylactically (e.g., for contrast dye allergy) or for the treatment of a non-autoimmune condition (e.g., delayed-type hypersensitivity reaction caused by contact allergen). (13) Any major surgical procedure requiring general anesthesia ≤28 days before randomization. Note: Diagnostic surgical procedures were not considered major surgical procedures, including but not limited to, tumor biopsy or endoscopic examinations regardless of the method of anesthesia. (14) Prior allogeneic stem cell transplantation or organ transplantation. (15) Clinically significant pericardial effusion. (16) Clinically uncontrolled pleural effusion or ascites that requires pleurocentesis or abdominal tapping for drainage within 2 weeks before randomization. (17) Any active malignancy ≤2 years before randomization except for the specific cancer under investigation in this study and any locally recurring cancer that has been treated curatively (e.g., resected basal or squamous cell skin cancer, superficial bladder cancer, carcinoma in situ of the cervix or breast). (18) Patients with untreated chronic hepatitis B virus (HBV), or chronic HBV carriers with HBV DNA ≥500 IU/mL (or >2500 copies/mL) at screening, or patients with active hepatitis C virus (HCV): (i) Patients with inactive hepatitis B surface antigen (HBsAg) or treated and stable hepatitis B (HBV DNA <500 IU/mL or <2500 copies/mL) were enrolled. The HBV DNA test was performed for patients who have a positive antibody to hepatitis B core antigen test. (ii) Patients with a negative HCV antibody test at screening or positive HCV antibody followed by a negative HCV RNA test at screening were eligible. The HCV RNA test was performed only for patients who test positive for HCV antibody test. Note: Patients with detectable HBsAg or detectable HBV DNA should be managed per treatment guidelines. Patients receiving antivirals at Screening should have been treated for >2 weeks prior to enrollment and should continue treatment for 6 months after drug treatment discontinues. (19) A known history of HIV infection. (20) Any of the following cardiovascular risk factors: a. Cardiac chest pain (defined as moderate pain that limits instrumental activities of daily living)≤28 days before randomization. b. Symptomatic pulmonary embolism ≤28 days before randomization. c. Any history of acute myocardial infarction ≤6 months before randomization. d. Any history of heart failure meeting New York Heart Association (NYHA) Classification III or IV ≤6 months before randomization. e. Any event of ventricular arrhythmia ≥Grade 2 in severity ≤6 months before randomization. f. Any history of cerebrovascular accident ≤6 months before randomization. g. Uncontrolled hypertension: systolic pressure ≥160 mmHg or diastolic pressure ≥100 mmHg despite anti-hypertension medications ≤28 days before randomization. h. Any episode of syncope or seizure ≤28 days before randomization. (21) Creatinine clearance <60 mL/min for cisplatin or <45 mL/min for carboplatin. (22) Patients with toxicities (as a result of prior anticancer therapy) which have not recovered to baseline or stabilized at the time of randomization, except for AEs not considered a likely safety risk (eg, alopecia, neuropathy and specific laboratory abnormalities). (23) A history of severe hypersensitivity reactions to other monoclonal antibodies. (24) A history of allergic reactions to cisplatin, carboplatin, or etoposide. (25) ≥Grade 2 peripheral neuropathy, as defined by NCI-CTCAE v5.0 criteria (cisplatin).

Renal Function Abnormalities: Patients with moderate renal dysfunction (estimated glomerular filtration rate >30 mL/min/1.73 m² and <60 mL/min/1.73 m² by Chronic Kidney Disease Epidemiology Collaboration equation) were permitted to enroll in the study. For patients with baseline renal insufficiency, the following algorithm was used for use of steroid treatment in the management of imAEs: (1) In the setting of a Grade 2 serum creatinine increase only, study treatment continue unless the serum creatinine increases by ≥50% from the baseline value OR the eGFR falls below 20 mL/min/1.73 m². (2) In the setting of a Grade 3 serum creatinine increase only, study treatment held until serum creatinine improves to baseline and treatment resume only after discussion with the medical monitor.

Concomitant Medications Procedures Prior medications were be defined as medications that were stopped before the day of first dose of study drug. Concomitant medications were defined as medications that 1) started before the first dose of study drug and were continuing at the time of the first dose of study drug, or 2) started on or after the day of the first dose of study drug up to 30 days after the patient's last dose (as of Safety Follow-up Visit).

### Permitted Concomitant Medications Procedures

Most concomitant medications and therapies that are in accordance with local standards of medical care and that are deemed necessary for supportive care (eg, anti-emetics, antidiarrheals) and in a patient's interest are allowed.

Systemic corticosteroids given for the control of imAEs was tapered gradually and at non-immunosuppressive doses (≤10 mg/day of prednisone or equivalent) before the next IMP administration. Short-term use of steroids as prophylactic treatments (eg, patients with contrast allergies to diagnostic imaging contrast dyes) is permitted.

Patients with active hepatitis B, defined as HBV DNA ≥500 IU/mL at screening, must initiate treatment 2 weeks before treatment and continue until 6 months after the last dose of study drug(s). Patients should continue effective antiviral treatment to decrease potential viral re-activation risk. Tenofovir and entecavir are recommended in the American Association for the Study of Liver Disease (AASLD) guideline because they lack resistance with long-term use (Terrault et al 2016; AASLD/IDSA HCV Guidance Panel, 2015). Other antiviral agents may be used as appropriate.

### Prohibited or Restricted Concomitant Medications/Procedures

The following medications were prohibited or restricted at the time of screening and during the administration of tislelizumab or placebo: (1) Immunosuppressive agents (except to treat a drug-related A). (2) Systemic corticosteroids >10 mg daily (prednisone or equivalent), except to treat or control a drug-related AL (per protocol), or as pretreatment for chemotherapy, or for short-term use as prophylactic treatment. (3) Live vaccines within 28 days before randomization and 60 days following the last dose of tislelizumab or placebo. (4) Herbal remedies or other agents with immune-stimulating properties (e.g., mistletoe extract) or that are known to potentially interfere with liver or other major organ functions (e.g., hypericin). (5) RANK-L inhibitors (eg, denosumab). Patients who were receiving denosumab prior to enrollment were required to be willing and eligible to receive a bisphosphonate instead.

TABLE 9
Selection and Timing of Dose Administration of Tislelizumab or Placebo in
Combination with Cisplatin and Etoposide

| Admini- | | | | Maintenance |
|---|---|---|---|---|
| stration | | Dose | | Period Cycle 5 |
| on Day 1 | Study Drug^{a} | (Route) | Induction Period Initial 4 Cycles^{b} | and Beyond |
| First | Tislelizumab | 200 | Day 1 of each 21-day cycle: | Day 1 of each |
| | or placebo | mg | Cycle 1: Infuse over 60 minutes (wait | 21-day cycle; |
| | | (IV) | ≥ 60 minutes before chemotherapy) | Infuse over 30 |
| | | | Cycles 2 to 4: Infuse over | minutes |
| | | | 30 minutes (wait ≥ 60 minutes | |
| | | | before chemotherapy at Cycle | |
| | | | 2 and thereafter wait ≥ 30 minutes | |
| | | | before chemotherapy) | |
| Second | Cisplatin | 75 | Day 1 of each 21-day cycle: Infuse | Discontinue |
| | | mg/m² | over 2 hours | Treatment |
| | | (IV) | | |
| Third | Etoposide | 100 | Day 1 to Day 3 of each 21-day cycle: | Discontinue |
| | | mg/m² | Infuse over 60 minutes | Treatment |
| | | (IV) | | |

Abbreviations:
IV, intravenous(ly);
SmPCs, summaries of product characteristics.
^{a}These products were prepared/stored/administered in accordance with the package inserts or SmPCs.
^{b}Reduction of tislelizumab or placebo infusion time from 60 minutes to 30 minutes was based on the 60-minute infusion time being well tolerated.

TABLE 10
Selection and Timing of Dose Administration of Tislelizumab or Placebo in
with Carboplatin and Etoposide

| Admini- | | | | Maintenance |
|---|---|---|---|---|
| stration | | Dose | | Period Cycle 5 |
| on Day 1 | Study Drug^{a} | (Route) | Induction Period Initial 4 Cycles^{b} | and Beyond |
| First | Tislelizumab | 200 mg | Day 1 of each 21-day cycle: | Day 1 of each |
| | or placebo | (IV) | Cycle 1: Infuse over 60 minutes (wait | 21-day cycle; |
| | | | ≥ 60 minutes before chemotherapy) | Infuse over 30 |
| | | | Cycles 2 to 4: Infuse over | minutes |
| | | | 30 minutes (wait ≥ 60 minutes | |
| | | | before chemotherapy at Cycle | |
| | | | 2 and thereafter wait ≥ 30 minutes | |
| | | | before chemotherapy) | |
| Second | Carboplatin^{c} | AUC 5 | Day 1 of each 21-day cycle: Infuse | Discontinue |
| | | (IV) | over 15-60 minutes to achieve an | Treatment |
| | | | AUC of 5 mg/mL/min (Calvert | |
| | | | formula dosing) | |
| Third | Etoposide | 100 | Day 1 to Day 3 of each 21-day cycle: | Discontinue |
| | | mg/m² | Infuse over 60 minutes | Treatment |
| | | (IV) | | |

Abbreviations:
IV, intravenous(ly);
SmPCs, summaries of product characteristics.
^{a}These products were prepared/stored/administered in accordance with the package inserts or SmPCs.
^{b}Reduction of tislelizumab or placebo infusion time from 60 minutes to 30 minutes was based on the 60-minute infusion time being well tolerated.
^{c}Calvert formula: Total dose (mg) = (target AUC) × (glomerular filtration rate [GFR] + 25).

GFR estimated by calculated creatinine clearance using Cockcroft-Gault Equation: Men: [(140−age (y))×weight (kg)]/[72×serum creatinine (mg/dL)]; Women: [(140−age (y))×weight (kg)]×0.85/[72×serum creatinine (mg/dL)].

TABLE 11
Demographics and Baseline Characteristics

| | Tislelizumab + | Placebo + | |
|---|---|---|---|
| | Chemotherapy | Chemotherapy | Total |
| | (N = 227) | (N = 230) | (N = 457) |
| | n (%) | n (%) | n (%) |
| Age (Years) | | | |
| n | 227 | 230 | 457 |
| Mean (SD) | 61.5 (7.75) | 60.7 (8.25) | 61.1(8.01) |
| Median | 63.0 | 62.0 | 62.0 |
| Q1, Q3 | 56.0, 66.0 | 55.0, 67.0 | 56.0, 67.0 |
| Min, Max | 31, 78 | 34, 78 | 31, 78 |
| Age Group, n (%) | | | |
| <65 Years | 138 (60.8) | 149 (64.8) | 287 (62.8) |
| >=65 Years | 89 (39.2) | 81 (35.2) | 170 (37.2) |
| Sex, n (%) | | | |
| Male | 186 (81.9) | 186 (80.9) | 372 (81.4) |
| Female | 41 (18.1) | 44 (19.1) | 85 (18.6) |
| BMI (kg/m\hat{ }2) | | | |
| n | 227 | 230 | 457 |
| Mean (SD) | 23.56 (3.139) | 23.34 (3.219) | 23.45 (3.178) |
| Median | 23.63 | 23.19 | 23.44 |
| Q1, Q3 | 21.11, 25.39 | 20.96, 25.10 | 21.08, 25.32 |
| Min, Max | 16.7, 36.6 | 16.1, 33.6 | 16.1, 36.6 |
| Baseline | | | |
| Weight (kg) | | | |
| n | 227 | 230 | 457 |
| Mean (SD) | 64.58 (10.043) | 63.98 (10.702) | 64.28 (10.373) |
| Median | 64.00 | 63.00 | 63.00 |
| Q1, Q3 | 57.50, 70.00 | 56.00, 70.00 | 57.00, 70.00 |
| Min, Max | 43.0, 96.0 | 43.0, 109.0 | 43.0, 109.0 |
| Smoking | | | |
| status, n (%) | | | |
| Never | 53 (23.3) | 59 (25.7) | 112 (24.5) |
| Current | 151 (66.5) | 135 (58.7) | 286 (62.6) |
| Former | 23 (10.1) | 36 (15.7) | 59 (12.9) |
| Baseline | | | |
| LDH, n (%) | | | |
| <=ULN | 114 (50.2) | 109 (47.4) | 223 (48.8) |
| >ULN | 113 (49.8) | 121 (52.6) | 234 (51.2) |
| ECOG | | | |
| Performance, n (%) | | | |
| 0 | 35 (15.4) | 34 (14.8) | 69 (15.1) |
| 1 | 192 (84.6) | 196 (85.2) | 388 (84.9) |
| Brain | | | |
| Metastases, n (%) | | | |
| Yes | 1 (0.4) | 4 (1.7) | 5 (1.1) |
| No | 226 (99.6) | 226 (98.3) | 452 (98.9) |
| Chemotherapy, | | | |
| n (%) | | | |
| Cisplatin | 47 (20.7) | 49 (21.3) | 96 (21.0) |
| Carboplatin | 180 (79.3) | 181 (78.7) | 361 (79.0) |

TABLE 12
Disease History

| | Tislelizumab + | Placebo + | Total |
|---|---|---|---|
| | Chemotherapy | Chemotherapy | (N = |
| | (N = 227) | (N = 230) | 457) |
| | n (%) | n (%) | n (%) |
| Time from | | | |
| Initial Diagnosis to | | | |
| Study Entry (Months) ^{a} | | | |
| N | 227 | 230 | 457 |
| Mean (SD) | 1.08 (3.064) | 2.02 (6.284) | 1.55 (4.970) |
| Median | 0.59 | 0.59 | 0.59 |
| Q1, Q3 | 0.43, 0.85 | 0.39, 0.82 | 0.39, 0.82 |
| Min, Max | 0.1, 34.9 | 0.1, 56.7 | 0.1, 56.7 |
| Stage at Initial | | | |
| Diagnosis, n (%) | | | |
| Limited-Stage Disease | 5 (2.2) | 12 (5.2) | 17 (3.7) |
| Extensive-Stage Disease | 222 (97.8) | 218 (94.8) | 440 (96.3) |
| AJCC Staging at Study | | | |
| Entry, n (%) ^{a} | | | |
| IIIA | 4 (1.8) | 2 (0.9) | 6 (1.3) |
| IIIB | 16 (7.0) | 27 (11.7) | 43 (9.4) |
| IV | 207 (91.2) | 201 (87.4) | 408 (89.3) |
| Distant Metastatic | | | |
| Site(s), n (%) ^{b} | | | |
| Liver | 64 (28.2) | 59 (25.7) | 123 (26.9) |
| Lung | 32 (14.1) | 59 (25.7) | 91 (19.9) |
| Brain | 1 (0.4) | 4 (1.7) | 5 (1.1) |
| Lymphnodes | 48 (21.1) | 45 (19.6) | 93 (20.4) |
| Number of Metastatic | | | |
| Sites, n (%) | | | |
| 1 | 2 (0.9) | 2 (0.9) | 4 (0.9) |
| 2 | 42 (18.5) | 64 (27.8) | 106 (23.2) |
| >=3 | 183 (80.6) | 164 (71.3) | 347 (75.9) |
| Target Lesion Sum | | | |
| of Diameter (mm) | | | |
| n | 227 | 228 | 455 |
| Mean (SD) | 110.27 (49.417) | 101.70 (47.121) | 105.98 (48.417) |
| Median | 104.30 | 94.00 | 99.40 |
| Q1, Q3 | 78.70, 137.00 | 66.00, 133.90 | 69.00, 135.20 |
| Min, Max | 16.9, 293.0 | 12.0, 243.3 | 12.0, 293.0 |

^{a} Study entry date is randomization date. AJCC staging was based on 7th version.
^{b} A patient could have multiple metastatic sites.

TABLE 13
Patient Disposition

| | Tislelizumab + | Placebo + |
|---|---|---|
| | Chemotherapy | Chemotherapy |
| | (n = 227) | (n = 230) |
| Randomized, n (%) | 227 (100.0) | 230 (100.0) |
| Chemotherapy allocation, n (%) | | |
| Cisplatin + etoposide | 47 (20.7) | 49 (21.3) |
| Carboplatin + etoposide | 180 (79.3) | 181 (78.7) |
| Treated, n (%) | 227 (100.0) | 229 (99.6) |
| Discontinued from study, n (%) | 167 (73.6) | 198 (86.1) |
| Reason for discontinuation | | |
| Death | 164 (72.2) | 192 (83.5) |
| Withdrawal by subject | 1 (0.4) | 4 (1.7) |
| Lost to follow-up | 2 (0.9) | 2 (0.9) |
| Median study follow-up, | 15.4 (0.2-44.9) | 13.2 (0.1-40.8) |
| months (range)^{a} | | |
| Median duration of | 19.4 (0.7-195.4) | 19.1 (3.0-148.9) |
| tislelizumab/placebo exposure, | | |
| weeks (range)^{b} | | |

^{a}Study follow-up time is defined as the time from the randomization date to the death date or end of study date (whichever occurs first) for patients discontinued from the study, or the database cutoff date for ongoing patients.
^{b}Duration of exposure is calculated as last dose date to first dose date + 1. For patients with treatment ongoing, last dose date = cutoff date; for patients discontinue the treatment, last dose date = min (cutoff date, death date, last dose date + 20).

### Methods

Analysis Sets: The ITT Analysis Set includes all randomized patients. Patients were analyzed according to their randomized treatment arms. This was the primary Analysis Set for all efficacy analyses. The Safety Analysis Set includes all randomized patients who received=1 dose of any component of study treatment; it is the Analysis Set for the safety analyses. Patients were analyzed according to the actual treatment regimen received. The PK Analysis Set includes all patients who received=1 dose of tislelizumab per the protocol, for whom any postdose PK data were available. The ADA Analysis Set includes all patients who were randomized to the tislelizumab arm and had a baseline and =1 postbaseline ADA result.

Disease Measurement: Response and progression were evaluated in this trial using the international criteria proposed by the Response Evaluation Criteria in Solid Tumors (RECIST) Committee (v1.1) (Eisenhauer EA, Therasse P, Bogaerts J, et al. New response evaluation criteria in solid tumors: revised RECIST guideline (version 1.1). Eur J Cancer. 2009; 45(2):228-47). Changes in only the largest diameter (uni-dimensional measurement) of the tumor lesions were used in the RECIST criteria. Tumor lesions were measured in at least 1 dimension (longest diameter in the plane of measurement recorded) with a minimum size of: (i) 10 mm by CT and MRI (no less than double the slice thickness and a minimum of 10 mm), (ii) 10 mm caliper measurement by clinical exam (when superficial), and (iii) 20 mm by chest X-ray (if clearly defined and surrounded by aerated lung).

Statistical Analysis: The p-value from a stratified log-rank test was presented using stratification factors with actual values as recorded in the electronic data capture (EDC) system at randomization. The OS hazard ratio (HR) between the two arms and its 95% confidence interval (CI) was estimated using a stratified Cox regression model with treatment arm as a factor and the model was stratified by the actual value of the stratification factors. The median OS and the cumulative probability of OS at 1 year and 2 years, was calculated for each treatment arm and presented with two-sided 95% CIs using Kaplan-Meier methodology. A Kaplan-Meier curve was constructed to provide a visual description of the difference between arms.

Patient Reported Outcome (PRO): The PRO instruments in this study are the European Organization for Research and Treatment of Cancer (EORTC) Quality of Life Questionnaire-Core 30 (QLQ-C30) that measures cancer patients' health status and general functionality and symptoms, EORTC supplemental lung cancer module (QLQ-LC13) that measures lung cancer specific symptoms, and the EuroQoL 5-Dimension, 5-Level (EQ5D-5L) that measures general health and wellbeing. Patients are asked to complete the EORTC-QLQ-C30, EORTC-QLQ-LC13, and EQ5D-5L questionnaires before any clinical activities are performed during on-study clinic visits.

Health-Related Quality of Life Domain: HRQoL is assessed via the EORTC QLQ-C30 GHS/QoL, functional and symptom scales scores, and the single item scores. Observed values and changes from baseline are summarized using descriptive statistics. Clinically meaningful changes post baseline (percentage of patients with a 10-point improvement or worsening; to be defined in the SAP if otherwise) for GHS and PF per the QLQ-C30 and dyspnoea, coughing, haemoptysis, dysphagia, chest pain, pain in arms and shoulders, and peripheral neuropathy symptoms of QLQ-LC13 is calculated by time point and compared between the treatment arms.

Biomarkers: Archival tumor tissues (formalin-fixed paraffin-embedded [FFPE] block or approximately 15 [5 6] unstained slides) are sent for immunohistochemistry assay (IHC) of PD-L1 status. In addition to PD-L1 expression, other predictive biomarkers, such as TMB and immune-mediated GEP, biomarkers expression by multiplex IHC, and other immune-mediated markers that are related to response or clinical benefit of tislelizumab are evaluated. If no archival samples are available, a fresh tumor biopsy at screening is strongly recommended, if feasible. For fresh biopsy specimens, acceptable samples include core needle biopsies for deep tumor tissue or excisional, incisional, punch, or forceps biopsies for cutaneous, subcutaneous, or mucosal lesions. In some embodiments, the elevated level of PD-L1 expression is at least 1%, 2%, 5%, 10%, 20%, or 50%.

Blood samples are taken at baseline (predose at Day 1 of Cycle 1) and optionally at the time of disease progression and/or at the time of first tumor response (CR/PR) (10 mL each timepoint) for all randomized patients to explore the association of blood-based biomarkers with response, resistance, and prognosis to tislelizumab in combination with chemotherapy or chemotherapy alone.

Distribution of PD-L1 expression is examined in the ITT Analysis Set. Any potential association between PD-L1 expression and tislelizumab treatment effect over control (PFS, OS, ORR, DOR, and DCR) is explored. Other potential predictive markers, including but not limited to GEP, TMB and MSI in archival and/or fresh tumor tissue and bTMB are assessed before study treatment and/or at disease progression. The association with disease status and/or response to tislelizumab in combination with chemotherapy is assessed.

### Results

### Brief Summary of Results

457 patients were randomized (tislelizumab arm, n=227; placebo arm, n=230). Baseline demographics were generally well balanced between treatment arms. Higher tumor burden was observed at baseline in the tislelizumab arm compared with the placebo arm; more patients had advanced disease (AJCC Stage IV: 91.2% vs 87.4%) and >3 metastatic lesions (80.6% vs 71.3%), respectively. Tislelizumab plus chemotherapy demonstrated a statistically significant OS benefit compared with placebo plus chemotherapy (stratified hazard ratio [HR]=0.75 [95% confidence interval (CI): 0.61, 0.92]; P=0.0035; median OS: 15.5 [95% CI: 13.5, 17.1] vs 13.5 months [95% CI: 12.1, 14.9], respectively).

OS rates at 1, 2, and 3 years were 62.7%, 33.2%, and 25.0%, respectively, in the tislelizumab arm and 58.4%, 22.4%, and 9.3%, respectively, in the placebo arm. Treatment with tislelizumab plus chemotherapy significantly improved PFS compared with placebo plus chemotherapy (stratified HR=0.63 [95% CI: 0.51, 0.78]; P<0.0001; median PFS: 4.8 [95% CI: 4.3, 5.5] vs 4.3 months [95% CI: 4.2, 4.4], respectively).

Further, improved confirmed ORR (68.3% vs 61.7%) and more durable responses (median DoR 4.3 vs 3.7 months) were observed in the tislelizumab vs placebo arm, respectively. In the tislelizumab arm, 59.9% of patients received ≥1 subsequent systemic anticancer therapy vs 73.9% in the placebo arm. In the safety analysis set, 85.5% of patients in the tislelizumab arm vs 86.0% in the placebo arm had ≥grade 3 treatment-related treatment-emergent adverse events (TRAEs), the most common (≥10% of patients) being hematologic and gastrointestinal toxicities in both arms. Serious TRAEs occurred in 31.3% of patients in the tislelizumab arm vs 17.9% in the placebo arm. Incidence of immune-mediated adverse events (imAEs) was 38.3% in the tislelizumab arm and 17.9% in the placebo arm. Most imAEs were manageable with systemic steroids or hormone therapies. In some embodiments, the subsequent cancer treatment is anlotinib. In some embodiments, tislelizumab is used in combination with anlotinib.

### Overall Survival

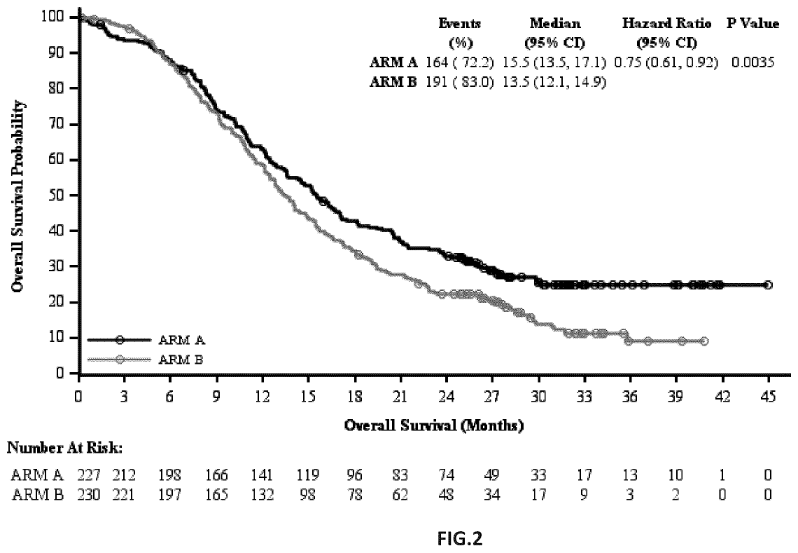

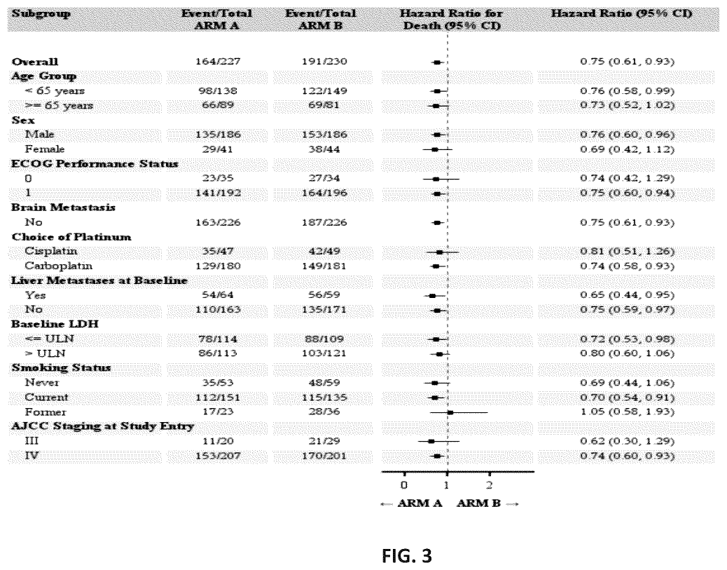

Improvement in overall survival (OS) is generally accepted as the best measure of clinical benefit for patients with advanced/unresectable or metastatic lung cancer. OS is defined as the time from randomization to death from any cause. OS was analyzed in the ITT Analysis Set. Data for patients who are not reported as having died at the time of analysis was censored at the date last known to be alive. Data for patients who did not have postbaseline information were censored at the date of randomization. OS was compared between tislelizumab+cisplatin or carboplatin+etoposide (Arm A) and placebo+cisplatin or carboplatin+etoposide (Arm B) in a stratified log-rank test using a one-sided significance level of 0.025 (Table 14, FIG. 2, FIG. 3): The null hypothesis to be tested is: H0: OS in Arm A≤OS in Arm B against the alternative hypothesis: Ha: OS in Arm A>OS in Arm B.

Overall survival demonstrated significant improvement in the patient population treated with Tislelizumab plus chemotherapy with a divergence in survival showing around 6 months and a clear and maintained separation at 9 months. (FIG. 2). Overall survival was improved consistently across all pre-defined subgroups treated with Tislelizumab plus chemotherapy compared to treatment with placebo plus chemotherapy (FIG. 3).

TABLE 14
Overall Survival

| | Tislelizumab + | Placebo + |
|---|---|---|
| | Chemotherapy | Chemotherapy |
| | (N = 227) | (N = 230) |
| Overall Survival | | |
| Death, n (%) | 164 (72.2) | 191 (83.0) |
| Censored, n (%) | 63 (27.8) | 39 (17.0) |
| Lost to Follow-up | 2 (0.9) | 2 (0.9) |
| Withdrew Consent | 1 (0.4) | 4 (1.7) |
| Ongoing Without Events | 60 (26.4) | 32 (13.9) |
| Other Reason | 0 (0.0) | 1 (0.4) |
| One-Sided Stratified Log-rank Test | 0.0035 | — |
| p-Value ^{a} | | |
| Stratified Hazard Ratio (95% CI) ^{ab} | 0.75 (0.61, 0.92) | — |
| Unstratified Hazard Ratio | 0.75 (0.61, 0.93) | — |
| (95% CI) ^{b} | | |
| Overall Survival (Months) | | |
| Median (95% CI) | 15.5 (13.5, 17.1) | 13.5 (12.1, 14.9) |
| Q1 (95% CI) | 8.9 (8.0, 10.3) | 8.4 (7.3, 9.4) |
| Q3 (95% CI) | 30.0 (25.9, NE) | 22.6 (19.4, 27.6) |
| Overall Survival Rate at, % (95% | | |
| CI) | | |
| 3 Months | 93.8 (89.8, 96.3) | 97.4 (94.2, 98.8) |
| 6 Months | 87.6 (82.6, 91.3) | 87.2 (82.1, 90.9) |
| 9 Months | 73.8 (67.6, 79.1) | 73.0 (66.7, 78.3) |
| 12 Months | 62.7 (56.1, 68.7) | 58.4 (51.7, 64.5) |
| 15 Months | 52.9 (46.2, 59.2) | 43.4 (36.8, 49.7) |
| 18 Months | 43.1 (36.6, 49.5) | 34.5 (28.4, 40.7) |
| 24 Months | 33.2 (27.1, 39.4) | 22.4 (17.2, 28.0) |
| 30 Months | 25.8 (20.0, 31.9) | 14.1 (9.4, 19.7) |
| Follow-up Time (Months) ^{e} | | |
| Median (95% CI) | 31.2 (28.2, 32.3) | 31.9 (27.8, 32.9) |

^{a} Stratified by ECOG performance (1 vs 0) and platinum (Carboplatin vs Cisplatin).
^{b} Hazard ratio and 95% CIs were estimated using a Cox regression model with placebo + chemotherapy as the reference group.
^{e} Median was estimated by the reverse Kaplan-Meier method with 95% CIs estimated using the Brookmeyer and Crowley method. Patient 086189-002 was with unknown death date and censored at last known alive date for overall survival analysis.

### Progression Free Survival

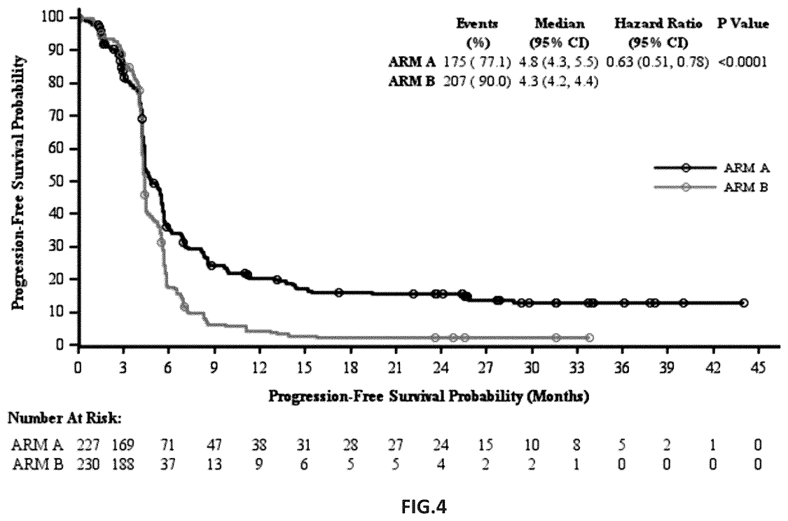

Progression-free survival (PFS) is defined as the time from randomization to the first documented disease progression by RECIST v1.1, or death from any cause, whichever occurred first. PFS was analyzed in the ITT Analysis Set (Table 15). PFS was calculated based on actual tumor assessment visit dates. Data for patients without disease progression or death at the time of analysis was censored at the time of the last valid tumor assessment. Data for patients without postbaseline tumor assessment was censored at the time of randomization. Data for patients who started to receive new anticancer therapy or were lost to follow-up were censored at the last valid tumor assessment date prior to the introduction of new therapy or loss to follow-up. Patients who had a clinical determination of progression underwent a radiographic imaging as required by RECIST v1.1, if possible, to correlate radiographic findings with the clinical findings. If a clinical determination of progression for a patient was confirmed, the date of the radiographic imaging was considered as the progression date for that patient. The hazard ratio (HR) for PFS was estimated using a stratified Cox regression model with treatment arm as a factor and stratified by the actual value of the stratification factors. The 95% confidence interval (CI) for the HR is provided. Kaplan-Meier methodology was used to estimate median PFS, cumulative probabilities of PFS at 6 month and 1-year, and a Kaplan-Meier curve was constructed to provide a visual description of the difference among arms (FIG. 4).

Tislelizumab plus chemotherapy significantly improved progression free survival in ES-SCLC patients compared to those treated with placebo plus chemotherapy.

TABLE 15
Progression Free Survival

| | Tislelizumab + | Placebo + |
|---|---|---|
| | Chemotherapy | Chemotherapy |
| | (N = 227) | (N = 230) |
| Progression-Free Survival | | |
| Events, n (%) | 175 (77.1) | 207 (90.0) |
| Progressive Disease | 162 (71.4) | 201 (87.4) |
| Death | 13 (5.7) | 6 (2.6) |
| Censored, n (%) | 52 (22.9) | 23 (10.0) |
| No Post-baseline | 6 (2.6) | 9 (3.9) |
| New Anticancer Therapy | 8 (3.5) | 4 (1.7) |
| Missed >= 2 Consecutive Assessments | 11 (4.8) | 4 (1.7) |
| Withdrew Consent | 0 (0.0) | 1 (0.4) |
| Ongoing Without Events | 27 (11.9) | 5 (2.2) |
| One-Sided Stratified Log-rank Test p- | <0.0001 | — |
| Value ^{a} | | |
| Stratified Hazard Ratio (95% CI) ^{ab} | 0.63 (0.51, 0.78) | — |
| Unstratified Hazard Ratio (95% CI) ^{b} | 0.63 (0.51, 0.77) | — |
| Progression-Free Survival (Months) | | |
| Median (95% CI) | 4.8 (4.3, 5.5) | 4.3 (4.2, 4.4) |
| Q1 (95% CI) | 4.0 (3.1, 4.1) | 4.0(3.8, 4.1) |
| Q3 (95% CI) | 8.5 (7.0, 13.7) | 5.7 (5.6, 5.7) |
| Event-Free Rate at, % (95% CI) | | |
| 3 Months | 81.8 (76.0, 86.4) | 86.7 (81.5, 90.6) |
| 6 Months | 35.4 (29.0, 42.0) | 17.7 (12.9, 23.1) |
| 9 Months | 24.3 (18.7, 30.4) | 6.5 (3.7, 10.4) |
| 12 Months | 20.7 (15.4, 26.5) | 4.5 (2.2, 8.0) |
| 18 Months | 16.2 (11.4, 21.7) | 2.5 (0.9, 5.4) |

^{a} Stratified by ECOG performance (1 vs 0) and platinum (Carboplatin vs Cisplatin).
^{b} Hazard ratio and 95% CIs were estimated using a Cox regression model with placebo + chemotherapy as the reference group.

### Overall Response Rate

Overall response rate (ORR) is the proportion of patients who had complete response (CR) or partial response (PR) per RECIST v1.1 in all randomized patients with measurable disease at baseline. Patients without any postbaseline assessment were considered non-responders. The difference in ORR between arms in the ITT Analysis Set was evaluated using the Cochran-Mantel-Haenszel (CMH) chi-square test with the actual stratification factors as strata for descriptive purpose only. The two-sided 95% CIs for the odds ratio and the difference in ORR were calculated, as well as Clopper-Pearson 95% CIs for the ORR within each arm.

Target lesions were evaluated using the following criteria: (1) Complete Response (CR): Disappearance of all target lesions. Any pathological lymph nodes (whether target or nontarget) must have reduction in short axis to <10 mm. (2) Partial Response (PR): At least a 30% decrease in the sum of diameters of target lesions, taking as reference the baseline sum diameters. (3) Progressive Disease (PD): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions was also considered progression). (4) Stable Disease: Neither sufficient shrinkage to qualify for PR nor sufficient increase to qualify for PD, taking as reference the smallest sum diameters while on study.

A higher objective response rate was observed with the Tislelizumab plus chemotherapy treatment than placebo plus chemotherapy (Table 16).

TABLE 16
Disease Response

| | Tislelizumab + | Placebo + |
|---|---|---|
| | Chemotherapy | Chemotherapy |
| | (N = 227) | (N = 230) |
| Best Overall Response, n (%) | | |
| Complete Response | 1 (0.4) | 0 (0.0) |
| Partial Response | 154 (67.8) | 142 (61.7) |
| Stable Disease | 46 (20.3) | 60 (26.1) |
| Non-CR/Non-PD | 0 (0.0) | 1 (0.4) |
| Progressive Disease | 11 (4.8) | 15 (6.5) |
| Could Not Be Determined | 15 (6.6) | 12 (5.2) |
| Not Evaluable ^{a} | 1 (0.4) | 0 (0.0) |
| Not Assessable ^{b} | 14 (6.2) | 12 (5.2) |
| Overall Response Rate, n (%) | 155 (68.3) | 142 (61.7) |
| 95% CI (%) ^{c} | 61.8, 74.3 | 55.1, 68.0 |
| Odds Ratio (95% CI) ^{d} | 1.33 (0.90, 1.96) | — |
| Risk Difference, % (95% CI) | 6.3 (−2.4, 15.0) | — |
| Disease Control Rate, n (%) ^{e} | 201 (88.5) | 203 (88.3) |
| 95% CI (%) ^{c} | 83.7, 92.4 | 83.4, 92.1 |
| Clinical Benefit Rate, n (%) ^{f} | 157 (69.2) | 150 (65.2) |
| 95% CI (%) ^{c} | 62.7, 75.1 | 58.7, 71.4 |

^{a} Patients who had >= 1 post-baseline tumor assessment, none of which were evaluable for response determination (e.g., not all target lesions captured). Not evaluable is based on RECIST 1.1.
^{b} Patients with no post-baseline tumor assessment by the data cutoff, including those who discontinued study (any reason) or died without having any post-baseline tumor assessment.
^{c} The 95% CI was estimated using the Clopper-Pearson method.
^{d} Odds ratio was calculated using Cochran-Mantel-Haenszel estimates and stratified by ECOG performance (1 vs 0) and platinum (Carboplatin vs Cisplatin).
^{e} Durable SD (SD with duration >= 12 weeks) was included.
^{f} Durable SD (SD with duration >= 24 weeks) was included.
Tumor response was evaluated based on RECIST1.1.

### Duration of Response

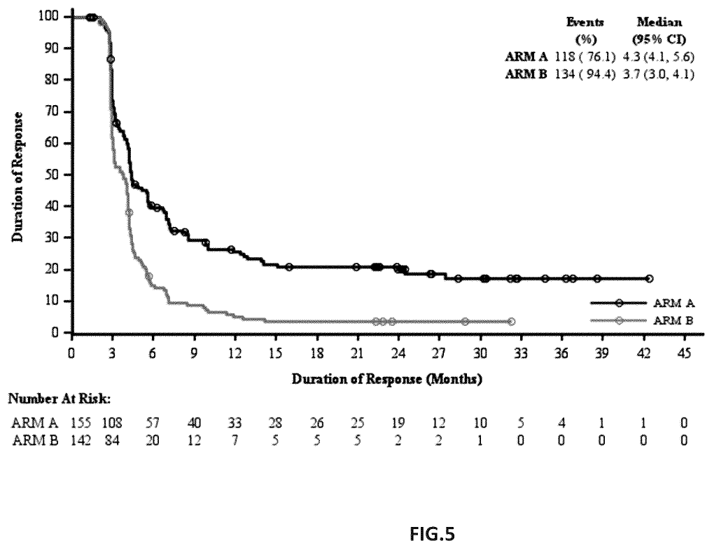

Duration of response (DOR) is defined for patients with an objective response as the time from the first documented objective response to documented disease progression as determined using the RECIST v1.1, or death from any cause, whichever occurs first. Data for patients who are alive and who have not experienced disease progression at the time of analysis was censored at the date of the last tumor assessment. If no tumor assessments was performed after the date of the first occurrence of the objective response (CR or PR), DOR was censored at the date of the first occurrence of the objective response. DOR was estimated using Kaplan-Meier methodology (FIG. 5). Comparisons between treatment arms was made using the stratified log-rank test for descriptive purposes only.

The antitumor response was stronger in the Tislelizumab plus chemotherapy arm than placebo plus chemotherapy (Table 17).

TABLE 17
Duration of Response

| | Tislelizumab + | Placebo + |
|---|---|---|
| | Chemotherapy | Chemotherapy |
| | (N = 227) | (N = 230) |
| Number of Responders | 155 | 142 |
| Duration of Response | | |
| Events, n (%) | 118 (76.1) | 134 (94.4) |
| Progressive Disease | 116 (74.8) | 134 (94.4) |
| Death | 2 (1.3) | 0 (0.0) |
| Censored, n (%) | 37 (23.9) | 8 (5.6) |
| New Anticancer Therapy | 5 (3.2) | 3 (2.1) |
| Missed >= 2 Consecutive Assessments | 5 (3.2) | 0 (0.0) |
| Ongoing Without Events | 27 (17.4) | 5 (3.5) |
| Duration of Response (Months) ^{a} | | |
| Median (95% CI) | 4.3 (4.1, 5.6) | 3.7 (3.0, 4.1) |
| Q1 (95% CI) | 2.9 (2.9, 3.2) | 2.8 (2.8, 2.9) |
| Q3 (95% CI) | 12.6 (8.1, 27.4) | 4.6 (4.3, 5.6) |

^{a} Medians and other quartiles were estimated using the Kaplan-Meier method with 95% CIs estimated using the Brookmeyer and Crowley method with log-log transformation
Tumor response was evaluated based on RECIST1.1.

### Subsequent Anticancer Therapies

Patients who discontinued study treatment early for reasons other than disease progression (e.g., toxicity) continued to undergo tumor assessments following the original plan until the patient began a subsequent anticancer treatment, experienced disease progression, withdrew consent, was lost to follow-up, death, or until the study terminated, whichever occurred first.

Subsequent anticancer treatment was more frequently and heavily administered in the placebo plus chemotherapy arm compared to the Tislelizumab plus chemotherapy arm, and across subsequent lines of therapy (Table 18).

TABLE 18
Subsequent Systemic Anticancer Therapies

| | Tislelizumab + | Placebo 1 | |
|---|---|---|---|
| | Chemotherapy | Chemotherapy | Total |
| | (N = 227) | (N II 230) | (N = 457) |
| | n (%) | n (%) | n (%) |
| Patients With Any Subsequent | 148 (65.2) | 180 (78.3) | 328 (71.8) |
| Systemic Anticancer Therapy | | | |
| Chemotherapy | 124 (54.6) | 155 (67.4) | 279 (61.1) |
| Immunotherapy | 43 (18.9) | 53 (23.0) | 96 (21.0) |
| Sintilimab | 12 (5.3) | 15 (6.5) | 27 (5.9) |
| Tislelizumab | 7 (3.1) | 12 (5.2) | 19 (4.2) |
| Durvalumab | 9 (4.0) | 5 (2.2) | 14 (3.1) |
| Atezolizumab | 8 (3.5) | 5 (2.2) | 13 (2.8) |
| Camrelizumab | 2 (0.9) | 7 (3.0) | 9 (2.0) |
| Monoclonal Antibodies | 2 (0.9) | 6 (2.6) | 8 (1.8) |
| Toripalimab | 2 (0.9) | 5 (2.2) | 7 (1.5) |
| Pembrolizumab | 1 (0.4) | 3 (1.3) | 4 (0.9) |
| Nivolumab | 1 (0.4) | 0 (0.0) | 1 (0.2) |
| Penpulimab | 1 (0.4) | 0 (0.0) | 1 (0.2) |
| Targeted Therapy | 69 (30.4) | 73 (31.7) | 142 (31.1) |
| Catequentinib (Anlotinib) | 65 (28.6) | 70 (30.4) | 135 (29.5) |
| Apatinib Mesylate | 3 (1.3) | 2 (0.9) | 5 (1.1) |
| Olaparib | 2 (0.9) | 1 (0.4) | 3 (0.7) |
| Other Protein Kinase Inhibitors | 0 (0.0) | 3 (1.3) | 3 (0.7) |
| Chiauranib | 2 (0.9) | 0 (0.0) | 2 (0.4) |
| Niraparib | 1 (0.4) | 1 (0.4) | 2 (0.4) |
| Other Antineoplastic Agents | 1 (0.4) | 1 (0.4) | 2 (0.4) |
| Fluzoparib | 0 (0.0) | 1 (0.4) | 1 (0.2) |
| Surufatinib | 1 (0.4) | 0 (0.0) | 1 (0.2) |
| Other Therapies | 45 (19.8) | 45 (19.6) | 90 (19.7) |
| Line of Treatment | | | |
| Second Line | 148 (65.2) | 180 (78.3) | 328 (71.8) |
| Third Line | 66 (29.1) | 83 (36.1) | 149 (32.6) |
| >Third Line | 21 (9.3) | 34 (14.8) | 55 (12.0) |

### Safety Summary

The safety profile of Tislelizumab plus chemotherapy was evaluated throughout the study. The safety profile was consistent with that of other PD-1/PD-L1 inhibitors when added to chemotherapy. The combination therapy of Tislelizumab plus chemotherapy showed an overall manageable safety profile.

TABLE 19
Safety Profile

| | | Tislelizumab + | Placebo + |
|---|---|---|---|
| | | Chemotherapy | Chemotherapy |
| TEAEs, n (%) | | (n = 227) | (n = 229) |
| Any grade, n (%) | | 226 (99.6) | 228 (99.6) |
| Grade ≥ 3 | | 201 (88.5) | 206 (90.0) |
| Serious, n (%) | | 94 (41.1) | 69 (30.1) |
| Leading to discontinuation, n (%) | | 30 (13.2) | 7 (3.1) |
| Leading to death, n (%) | | 14 (6.2) | 4 (1.7) |
| Most frequent | Anemia | 192 (84.6) | 194 (84.7) |
| TEAEs by preferred | Alopecia | 179 (78.9) | 181 (79.0) |
| term, n (%) | Neutropenia | 156 (68.7) | 161 (70.3) |

Immune-mediated AEs, n (%)

| | | |
|---|---|---|
| Any grade | 87 (38.3) | 41 (17.9) |
| Grade ≥ 3 | 25 (11.0) | 1 (0.4) |
| Serious | 22 (9.7) | 3 (1.3) |
| Leading to discontinuation | 13 (5.7) | 2 (0.9) |
| Leading to death | 1 (0.4) | 0 (0.0) |

Abbreviations:
TEAE, treatment-emergent adverse eventClinical Trial Efficacy Conclusions

This study demonstrated Tislelizumab plus chemotherapy significantly improved overall survival and progression free survival versus placebo plus chemotherapy in the treatment of ES-SCLC (P=0.0035 and P<0.0001, respectively). First, Tislelizumab plus chemotherapy has demonstrated statistically significant and clinically meaningful benefit in overall survival at final analysis with median study follow up of 14.1 months (minimal follow up defined by last patient randomized to the data cut off is 23.9 months). Median overall survival was 15.5 months with Tislelizumab plus chemotherapy versus 13.5 months with placebo plus chemotherapy (HR 0.75 [95% CI: 0.61, 0.92]; p=0.0035). The overall survival rate at 24 months was 33.2% versus 22.4% with placebo plus chemotherapy. The overall survival benefit was consistently observed across all the pre-defined subgroups.

Tislelizumab plus chemotherapy also significantly improved progression free survival. The median progression free survival was 4.8 months versus 4.3 months, respectively (HR 0.63 [95% CI: 0.51, 0.78]; p<0.0001). The safety profile in the safety analysis was consistent with the known risks of tislelizumab and chemotherapy and the underlying condition of the disease. The combination of tislelizumab and chemotherapy showed a manageable and acceptable safety and tolerability profile in the first line of treatment of patients with ES-SCLC, with no new safety signals. The results from this Phase 3 study support Tislelizumab plus chemotherapy as a first line treatment for patients with ES-SCLC.

### Example 2: Follow Up Study after Data Cutoff of Trial Described in Example 1

The purpose of this example is to detail a follow up study conducted subsequent to the data cutoff time point of the trial detailed in Example 1.

At the final analysis data cutoff of the Study described in Example 1 above, median study follow-up time in the intent-to-treat (ITT) population (from randomization to death, discontinuation from the study, or the data cutoff, whichever occurred first) was 14.2 months (IQR 8.6-25.3); and minimum study follow-up time (from last patient randomization to data cutoff) was 23.9 months.

Overall, 456 (>99%) randomized patients received any dose of their randomized study treatment. Mean duration of treatment was higher for tislelizumab (39.0 weeks [standard deviation (SD) 43.2]) than placebo (23.6 weeks [SD 19.0]), although median duration of treatment was similar (19.4 weeks [IQR 15.4-41.1] and 19.1 weeks [IQR 17.3-25.7], respectively); more patients in the tislelizumab arm received >16 cycles (≥1 year) of study treatment (Table 13). At the final analysis data cutoff, tislelizumab treatment was ongoing in 24 of 227 patients (11%) and placebo treatment was ongoing in five of 230 patients (2%). Chemotherapy exposure was similar in the two treatment arms (median of four cycles in both arms) (Table 20).

TABLE 20
Study Treatment Exposure (Safety Analysis Set)

| | Tislelizumab plus | Placebo plus | Tislelizumab plus | | Placebo plus | | | |
|---|---|---|---|---|---|---|---|---|
| | chemotherapy | chemotherapy | chemotherapy | | chemotherapy | | | |
| | (n = 227) | (n = 229) | (n = 227) | | (n = 229) | | | |
| | Tislelizumab | Placebo | Cisplatin | Carboplatin | Etoposide | Cisplatin | Carboplatin | Etoposide |
| Patients | 227 | 229 | 47 | 183 | 227 | 49 | 180 | 229 |
| exposed, n | (100%) | (100%) | (21%)* | (81%)* | (100%) | (21%) | (79%) | (100%) |
| (%) | | | | | | | | |
| Duration | | | | | | | | |
| of | | | | | | | | |
| exposure, | | | | | | | | |
| weeks^{†} | | | | | | | | |
| Mean (SD) | 39.0 | 23.6 | 11.3 | 11.9 | 12.0 | 12.4 | 12.1 | 12.2 |
| | (43.2) | (19.0) | (3.5) | (3.3) | (3.1) | (3.5) | (3.0) | (3.1) |
| Median | 19.4 | 19.1 | 12.4 | 12.4 | 12.4 | 12.3 | 12.6 | 12.6 |
| (IQR) | (15.4- | (17.3- | (12.0- | (12.0- | (12.0- | (12.0- | (12.0- | (12.0- |
| | 41.1) | 25.7) | 13.4) | 13.1) | 13.3) | 13.1) | 13.3) | 13.1) |
| Relative | | | | | | | | |
| dose | | | | | | | | |
| intensity, | | | | | | | | |
| %^{‡} | | | | | | | | |
| Mean (SD) | 92.4 | 93.4 | 90.4 | 88.9 | 87.3 | 88.6 | 88.4 | 87.5 |
| | (8.9) | (7.4) | (8.5) | (13.1) | (12.8) | (12.4) | (11.6) | (11.9) |
| Number of | | | | | | | | |
| cycles | | | | | | | | |
| received | | | | | | | | |
| Mean, n | 11.8 | 7.3 | 3.5 | 3.6 | 3.7 | 3.7 | 3.7 | 3.7 |
| (SD) | (13.0) | (5.7) | (1.0) | (0.9) | (0.8) | (0.8) | (0.8) | (0.8) |
| Median, n | 6.0 | 6.0 | 4.0 | 4.0 | 4.0 | 4.0 | 4.0 | 4.0 |
| (IQR) | (5-13) | (5-8) | (4-4) | (4-4) | (4-4) | (4-4) | (4-4) | (4-4) |
| >16 | 44 | 10 | NA | NA | NA | NA | NA | NA |
| cycles, n | (19%) | (4%) | | | | | | |
| (%) | | | | | | | | |

*Three patients in the tislelizumab arm who were unable to tolerate cisplatin treatment were switched to carboplatin; data from these patients are included in both the cisplatin and carboplatin columns in this table.
^{†}Duration of exposure was calculated as last dose date − first dose date + 1 day. For patients with treatment ongoing, last dose date = cutoff date; for patients who discontinued tislelizumab/placebo, last dose date = min (cutoff date, death date, last dose date + 20); for patients who discontinued treatment with cisplatin or carboplatin, last dose date = min (cutoff date, death date, last dose date + 20 days); for patients who discontinued the treatment of etoposide, last dose date = min (cutoff date, death date, last cycle first dose date + 20 days).
^{‡}Relative dose intensity was defined as the ratio of the actual dose intensity and the planned dose intensity.
IQR, interquartile range; NA, not applicable; SD, standard deviation.

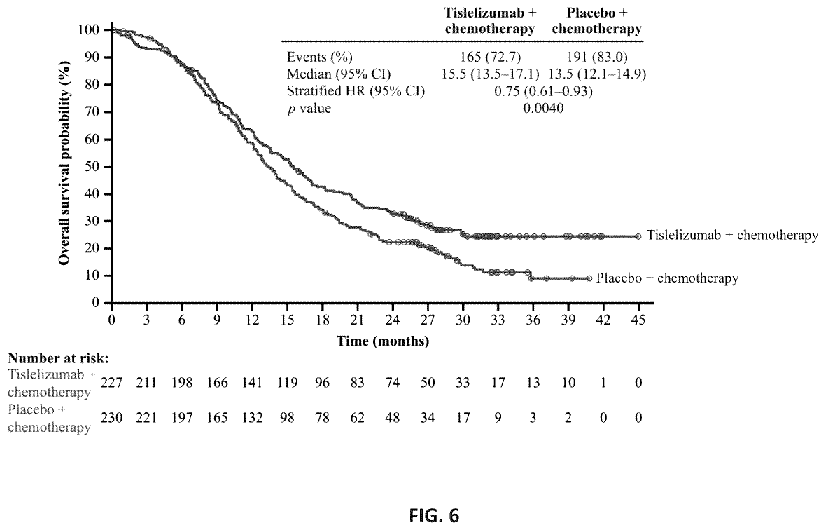

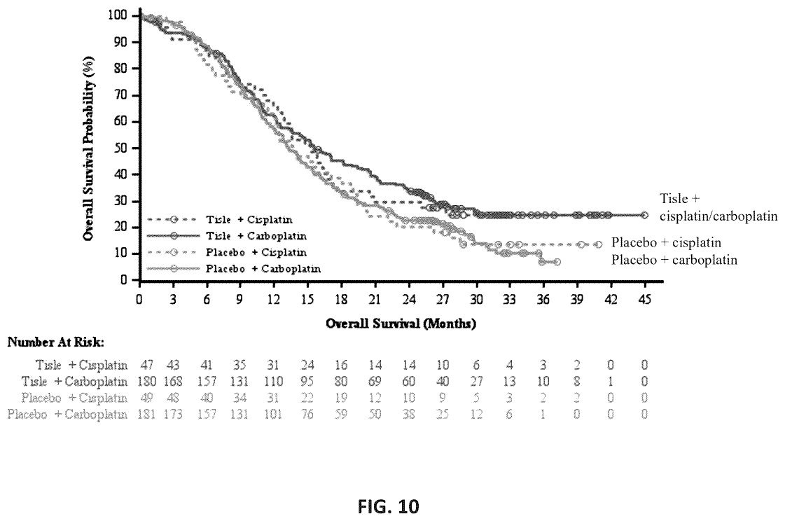

At data cutoff, a total of 356 deaths were observed in the ITT population, including 165 of 227 patients (73%) in the tislelizumab arm and 191 of 230 patients (83%) in the placebo arm. The primary study endpoint was met, with tislelizumab plus chemotherapy demonstrating a statistically significant improvement in OS versus placebo plus chemotherapy in the ITT population (stratified HR=0.75 [95% CI: 0.61-0.93]; p=0.0040) (FIGS. 6 and 10). Median OS was 15.5 months (95% CI: 13.5-17.1) in the tislelizumab arm and 13.5 months (95% CI: 12.1-14.9) in the placebo arm. Estimated 1-, 2-, and 3-year OS rates in the tislelizumab versus placebo arms were 63% versus 58%, 33% versus 22%, and 25% versus 9%, respectively.

A restricted mean survival time (RMST) analysis was carried out to account for the possible non-proportional hazards of OS and was computed using the area under the curve from baseline to the minimum of the largest observed time on each of the two treatment groups with a cutoff at 40.8 months. The RMST analysis for OS, with a cutoff at 40.8 months (the minimum of the longest follow-up time in either treatment arm), demonstrated a 3.2-month difference in OS (95% CI: 0.9-5.5) in favor of tislelizumab plus chemotherapy, indicative of clinical benefit compared with placebo plus chemotherapy. Prespecified multivariate analysis adjusting for liver metastasis, baseline LDH, and smoking status supported the primary analysis, with a stratified HR of 0.72 (95% CI: 0.58-0.89). Additionally, a post-hoc multivariate analysis adjusting for the observed imbalance in number of distant metastatic sites as an additional covariate further supported the positive treatment effect in the tislelizumab contained arm, with a stratified HR of 0.71 (95% CI: 0.57-0.87). An OS benefit of tislelizumab plus chemotherapy versus placebo plus chemotherapy was consistently seen across patient subgroups (FIG. 3). The RSMT analysis of OS favored tislelizumab plus chemotherapy with a larger difference observed in the present studies (3.2 months) compared with prior studies (1.2-month difference with pembrolizumab plus chemotherapy versus placebo plus chemotherapy; and 2.3-month difference with adebrelimab plus chemotherapy versus placebo plus chemotherapy).

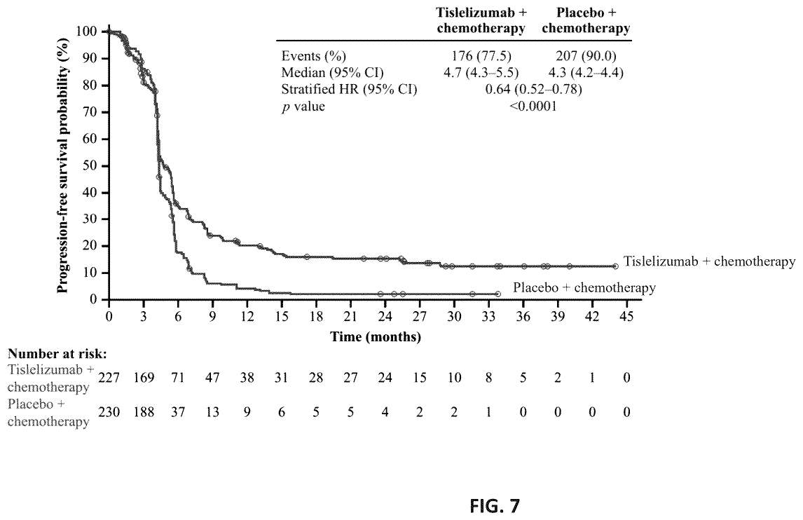

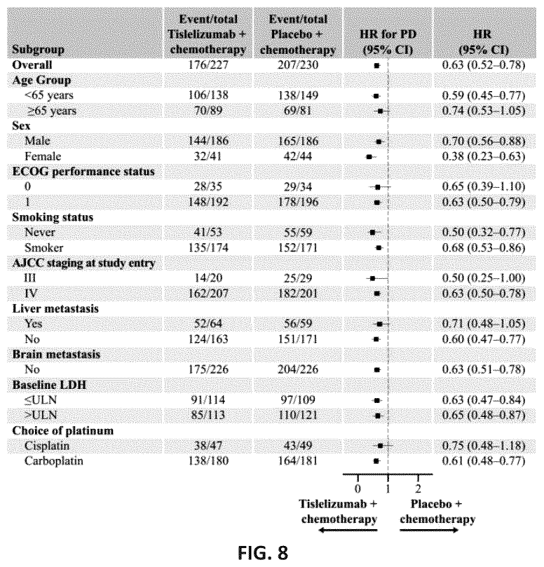

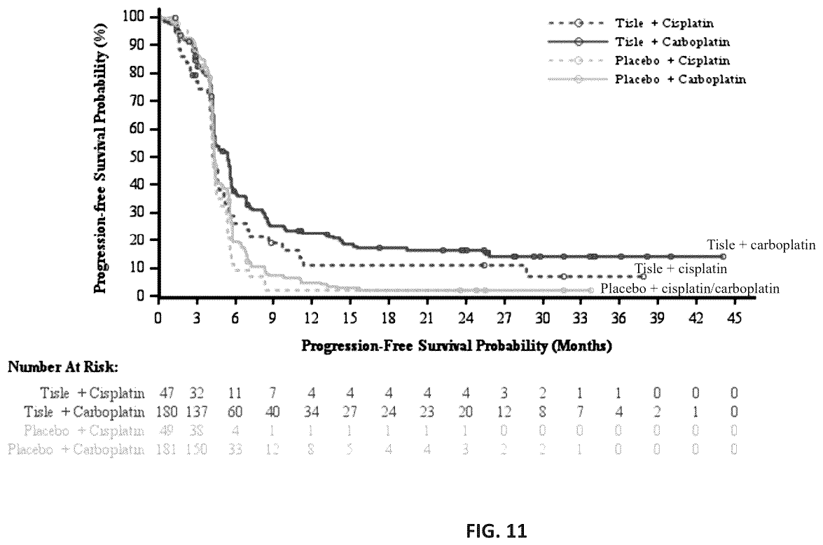

Tislelizumab plus chemotherapy significantly improved PFS versus placebo plus chemotherapy (PFS events; 176 patients [78%] in the tislelizumab arm versus 207 patients [90%] in the placebo arm; stratified HR=0.64 [95% CI: 0.52-0.78]; one-sided p<0.0001) (FIGS. 7 and 11). Median PFS was 4.7 months (95% CI: 4.3-5.5) in the tislelizumab arm and 4.3 months (95% CI: 4.2-4.4) in the placebo arm. Estimated 6- and 12-month PFS rates in the tislelizumab versus placebo arms, were 35% versus 18%, respectively, and 21% versus 5%, respectively. A PFS benefit of tislelizumab plus chemotherapy versus placebo plus chemotherapy was consistently seen across patient subgroups (FIG. 8).

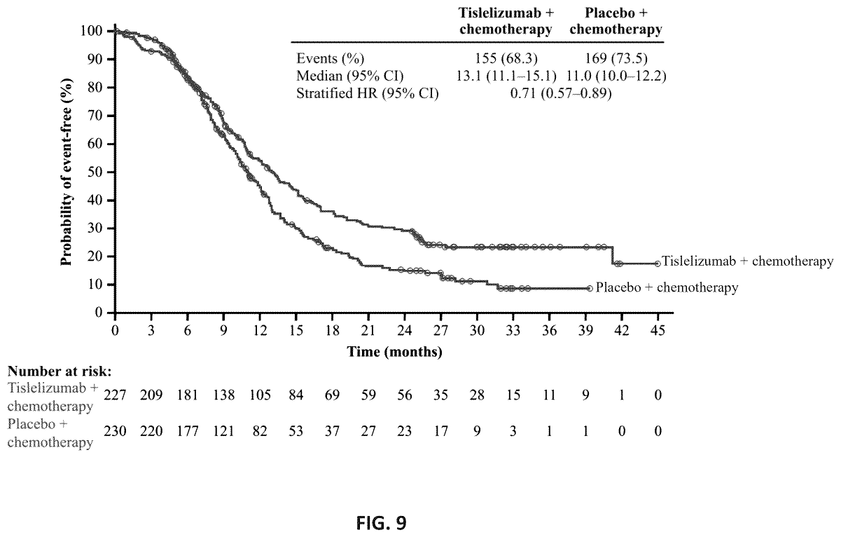

Improved PFS following next line of treatment after discontinuation of study drugs was observed in the tislelizumab arm (PFS2 stratified HR=0.71 [95% CI: 0.57-0.89]; median PFS2: 13.1 months versus 11.0 months in the tislelizumab and placebo arm, respectively), indicating a sustained benefit with tislelizumab beyond disease progression despite the more frequent use of second line subsequent anticancer treatment in the placebo arm (FIG. 9).

The confirmed ORR was numerically greater in the tislelizumab arm (68% [95% CI: 62-74]) versus the placebo arm (62% [95% CI: 55-68]) (Table 9). The median DoR was numerically longer with tislelizumab plus chemotherapy (4.3 months [95% CI: 4.1-5.6]) compared with placebo plus chemotherapy (3.7 months [95% CI: 3.0-4.1]) (Table 9).

A total of 306 patients (67%) in the ITT population received subsequent systemic anticancer therapies after discontinuation of study drugs (136 patients [60%] in the tislelizumab arm; 170 patients [74%] in the placebo arm). The most commonly used subsequent systemic anticancer therapies in both arms were conventional chemotherapy (124 patients [55%] in the tislelizumab arm; 155 patients [67%] in the placebo arm), immunotherapy (43 patients [19%] in the tislelizumab arm; 53 patients [23%] in the placebo arm) and targeted therapy (69 patients [30%] in the tislelizumab arm; 73 patients [32%] in the placebo arm), with more patients in the placebo arm receiving multiple lines of subsequent anticancer therapy (Table 18).

Treatment-related AEs (TRAEs) were reported in 226 of 227 patients (>99%) in the tislelizumab arm and in 228 of 229 patients (>99%) in the placebo arm (Table 21). Grade ≥3 TRAEs occurred in 194 patients (86%) in the tislelizumab arm and in 197 patients (86%) in the placebo arm, and were mostly hematological events, including neutropenia (in 127 patients [56%] and 125 patients [55%], respectively), and decreased white blood cell count (in 54 patients [24%] and 63 patients [28%], respectively) (Table 21).

TABLE 21
Treatment-Related Adverse Events by
Preferred Term (Safety Analysis Set)

| | Tislelizumab plus | | Placebo plus | |
|---|---|---|---|---|
| | chemotherapy | | chemotherapy | |
| | (n = 227) | | (n = 229) | |
| | Any grade | Grade ≥ 3 | Any grade | Grade ≥ 3 |
| Any | 226 (>99%) | 194 (85%) | 228 (>99%) | 197 (86%) |
| Alopecia | 177 (78%) | 0 (0%) | 182 (79%) | 4 (2%) |
| Anemia | 172 (76%) | 37 (16%) | 180 (79%) | 38 (17%) |
| Neutropenia | 156 (69%) | 127 (56%) | 161 (70%) | 125 (55%) |
| WBC count | 126 (56%) | 54 (24%) | 148 (65%) | 63 (27%) |
| decreased | | | | |
| Thrombo- | 112 (49%) | 44 (19%) | 119 (52%) | 58 (25%) |
| cytopenia | | | | |
| Nausea | 92 (41%) | 1(<1%) | 93 (41%) | 1(<1%) |
| Leukopenia | 75 (33%) | 26 (11%) | 56 (24%) | 24 (10%) |
| Decreased | 73 (32%) | 1(<1%) | 76 (33%) | 0 (0%) |
| appetite | | | | |
| ALT increased | 60 (26%) | 3 (1%) | 40 (17%) | 1(<1%) |
| Constipation | 50 (22%) | 0 (0%) | 27 (12%) | 0 (0%) |
| Neutrophil | 50 (22%) | 33 (14%) | 56 (24%) | 49 (21%) |
| count decreased | | | | |
| Vomiting | 48 (21%) | 1(<1%) | 48 (21%) | 0 (0%) |
| AST increased | 43 (19%) | 3 (1%) | 31 (13%) | 0 (0%) |
| Platelet count | 30 (13%) | 8 (3%) | 34 (15%) | 8 (3%) |
| decreased | | | | |
| Hypothyroidism | 29 (13%) | 1(<1%) | 9 (4%) | 0 (0%) |
| Rash | 28 (12%) | 5 (2%) | 18 (8%) | 0 (0%) |
| Malaise | 26 (11%) | 4 (2%) | 24 (10%) | 0 (0%) |
| Hypo- | 24 (11%) | 0 (0%) | 17 (7%) | 0 (0%) |
| albuminemia | | | | |
| Fatigue | 23 (10%) | 3 (1%) | 12 (5%) | 0 (0%) |
| Hyponatremia | 23 (10%) | 4 (2%) | 14 (6%) | 3 (1%) |
| Lymphocyte | 23 (10%) | 7 (3%) | 23 (10%) | 8 (3%) |
| count decreased | | | | |

TRAEs led to discontinuation of therapy in 25 patients (11%) and four patients (2%) in the tislelizumab and placebo arms, respectively (Table 22). Treatment-related serious AEs were reported in 71 patients (31%) and 41 patients (18%) in the tislelizumab arm and placebo arm, respectively (Table 22); all events with an incidence of 2% or more per arm were hematological. Eight patients (4%) in the tislelizumab arm experienced a TRAE leading to death (Table 22), including five events assessed by investigator as related to both tislelizumab and chemotherapy (thrombocytopenia, acute cardiac failure, autoimmune myocarditis, respiratory failure, and death with unknown cause), two assessed as only related to tislelizumab (gastrointestinal hemorrhage and depressed level of consciousness), and one assessed as only related to chemotherapy (febrile neutropenia). Half of the patients with TRAEs leading to death were >60 years old and most had indicators of high tumor burden at baseline (i.e., AJCC stage IV disease with three or more metastatic sites at study entry). No patients in the placebo arm experienced a TRAE leading to death.

TABLE 22
Summary of Treatment-Emergent Adverse Event and Treatment-
Related Adverse Event Incidence (Safety Analysis Set)

| | Tislelizumab plus | Placebo plus |
|---|---|---|
| | chemotherapy | chemotherapy |
| | (n = 227) | (n = 229) |
| Any TEAE | 226 (>99%) | 228 (>99%) |
| Grade ≥3 TEAE | 201 (89%) | 206 (90%) |
| Any serious TEAE | 94 (41%) | 69 (30%) |
| TEAE leading to death | 14 (6%) | 4 (2%) |
| TEAE leading to treatment | 30 (13%) | 7 (3%) |
| discontinuation | | |
| Leading to tislelizumab/placebo | 26 (11%) | 6 (3%) |
| discontinuation | | |
| Leading to chemotherapy | 16 (7%) | 5 (2%) |
| discontinuation | | |
| TEAE leading to dose modification* | 155 (68%) | 153 (67%) |
| TEAE leading to tislelizumab/ | 108 (48%) | 100 (44%) |
| placebo dose modification* | | |
| TEAE leading to chemotherapy | 132 (58%) | 138 (60%) |
| dose modification* | | |
| Any TRAE | 226 (>99%) | 228 (>99%) |
| Grade ≥3 TRAE | 194 (85%) | 197 (86%) |
| Any serious TRAE | 71 (31%) | 41 (18%) |
| TRAE leading to death | 8 (4%) | 0 (0%) |
| TRAE leading to treatment | 25 (11%) | 4 (2%) |
| discontinuation | | |
| Leading to tislelizumab/placebo | 21 (9%) | 3 (1%) |
| discontinuation | | |
| Leading to chemotherapy | 12 (5%) | 2 (<1%) |
| discontinuation | | |
| TRAE leading to dose modification* | 144 (63%) | 145 (63%) |
| TRAE leading to tislelizumab/ | 95 (42%) | 89 (39%) |
| placebo dose modification* | | |
| TRAE leading to chemotherapy dose | 126 (56%) | 134 (59%) |
| modification* | | |
| Immune-mediated AEs | 87 (38%) | 41 (18%) |
| Grade ≥3 imAE | 25 (11.0) | 1 (0.4) |
| imAE leading to death | 1 (0.4) | 0 (0.0) |

Immune-mediated AEs (imAE) of any grade were reported in 87 patients (38%) in the tislelizumab arm and in 41 patients (18%) in the placebo arm, with hypothyroidism, rash, and hyperthyroidism most commonly reported (Table 23). Grade 3 or worse imAEs were reported in 25 patients (11%) and one patient (<1%) in the tislelizumab and placebo arms, respectively (Table 22). One death due to an imAE was reported in the study (the autoimmune myocarditis case mentioned previously, in the tislelizumab arm).

TABLE 23
Most Common (≥1% of Patients in Either Treatment Arm)
Immune-Mediated Adverse Events (Safety Analysis Set)

| | Tislelizumab plus | | Placebo plus | |
|---|---|---|---|---|
| | chemotherapy | | chemotherapy | |
| | (n = 227) | | (n = 229) | |
| | Grade | | Grade | |
| Patients, n (%) | Any | ≥3 | Any | ≥3 |
| Hypothyroidism | 31 (14%) | 1 (<1%) | 9 (4%) | 0 (0%) |
| Rash | 30 (13%) | 5 (2%) | 18 (8%) | 0 (0%) |
| Hyperthyroidism | 13 (6%) | 0 (0%) | 2 (<1%) | 0 (0%) |
| Pneumonitis | 10 (4%) | 4 (2%) | 5 (2%) | 0 (0%) |
| Diabetes mellitus | 4 (2%) | 3 (1%) | 0 (0%) | 0 (0%) |
| Immune-mediated | 4 (2%) | 0 (0%) | 0 (0%) | 0 (0%) |
| myocarditis | | | | |
| Immune-mediated | 3 (1%) | 2 (<1%) | 0 (0%) | 0 (0%) |
| hepatitis | | | | |
| Immune-mediated lung | 3 (1%) | 1 (<1%) | 1 (<1%) | 0 (0%) |
| disease | | | | |
| Myositis | 3 (1%) | 0 (0%) | 1 (<1%) | 0 (0%) |
| Thyroxine-free decrease | 3 (1%) | 0 (0%) | 0 (0%) | 0 (0%) |
| Thyroxine increase | 1 (<1%) | 0 (0%) | 4 (2%) | 0 (0%) |

Patients with Long-Term Progression-Free Survival

Some patients received tislelizumab long-term. The baseline characteristics of 24 patients receiving long-term tislelizumab treatment were generally comparable with that in the overall population. Among them, 20 patients (83%) received tislelizumab for more than 2 years (≥35 cycles), of which six patients were administered with tislelizumab for more than 3 years (≥52 cycles).

Improved PFS following next line of treatment after discontinuation of study drugs was observed in the tislelizumab arm (PFS2 stratified HTR=0.71 [9500 CI: 0.57-0.89]; median PFS2: 13.1 months versus 11.0 months in the tislelizumab and placebo arm, respectively), indicating a sustained benefit with tislelizumab beyond disease progression despite the more frequent use of second line subsequent anticancer treatment in the placebo arm (FIG. 9).

The characteristics of patients with long-term are shown in Table 24 below.

TABLE 24
Baseline characteristics for patients with long-term progressionfree
survival (≥6 months)

| | Tislelizumab plus | Placebo plus | |
|---|---|---|---|
| | chemotherapy | chemotherapy | Total |
| | (n = 71) | (n = 37) | (N = 108) |
| Age, years | | | |
| Median (IQR) | 63.0 | 63.0 | 63.0 |
| <65 years | 44 (62.0) | 23 (62.2) | 67 (62.0) |
| ≥65 years | 27 (38.0) | 14 (37.8) | 41 (38.0) |
| Sex | | | |
| Male | 58 (81.7) | 33 (89.2) | 91 (84.3) |
| Female | 13 (18.3) | 4 (10.8) | 17 (15.7) |
| ECOG | | | |
| performance status | | | |
| 0 | 12 (16.9) | 6 (16.2) | 18 (16.7) |
| 1 | 59 (83.1) | 31 (83.8) | 90 (83.3) |
| Smoking status | | | |
| Never | 15 (21.1) | 7 (18.9) | 22 (20.4) |
| Current | 47 (66.2) | 22 (59.5) | 69 (63.9) |
| Former | 9 (12.7) | 8 (21.6) | 17 (15.7) |
| AJCC stage at | | | |
| study entry*^{,†} | | | |
| IIIA | 2 (2.8) | 1 (2.7) | 3 (2.8) |
| IIIB | 8 (11.3) | 7 (18.9) | 15 (13.9) |
| IV | 61 (85.9) | 29 (78.4) | 90 (83.3) |
| Number of | | | |
| metastatic sites^{‡} | | | |
| 1 | 1 (1.4) | 0 (0.0) | 1 (0.9) |
| 2 | 16 (22.5) | 16 (43.2) | 32 (29.6) |
| ≥3 | 54 (76.1) | 21 (56.8) | 75 (69.4) |
| Liver Metastasis | | | |
| Yes | 12 (16.9) | 3 (8.1) | 15 (13.9) |
| No | 59 (83.1) | 34 (91.9) | 171 (74%) |
| Brain Metastasis | | | |
| Yes | 0 (0.0) | 1 (2.7) | 1 (0.9) |
| No | 71 (100.0) | 36 (97.3) | 107 (99.1) |
| Baseline LDH | | | |
| ≤ULN | 39 (54.9) | 18 (48.6) | 57 (52.8) |
| >ULN | 32 (45.1) | 19 (51.4) | 51 (47.2) |
| Choice of | | | |
| Platinum | | | |
| Carboplatin | 60 (84.5) | 33 (89.2) | 93 (86.1) |
| Cisplatin | 11 (15.5) | 4 (10.8) | 15 (13.9) |

Data are n (%) unless stated otherwise.
*Study entry was date of randomization.
^{†}Based on AJCC Staging Manual Seventh Edition.
^{‡}A patient could have multiple metastatic sites.
AJCC, American Joint Committee on Cancer;
ECOG, Eastern Cooperative
Oncology Group;
IQR, interquartile range;
LDH, lactate dehydrogenase;
ULN, upper limit of normal.

Discussion. The study met its primary endpoint and demonstrated superior OS with tislelizumab versus placebo when added to platinum plus etoposide as first-line therapy for ES-SCLC (HR of 0.75 [95% CI: 0.61-0.93]). The OS benefit with tislelizumab plus chemotherapy was consistently observed across all examined patient subgroups and was accompanied by a statistically significant improvement in PFS, a greater ORR, and more durable responses versus placebo plus chemotherapy.

As of the data cutoff, the OS data represents a robust analysis with greater maturity, demonstrated by a longer-follow up (minimum 23.9 months), and a higher event-to-patient ratio (78%) than in prior phase 3 trials reporting OS benefit with addition of anti-PD-(L)1 treatment to chemotherapy in ES-SCLC, acknowledging the caveat of cross-study comparison. Long-lasting treatment benefit was observed throughout the study follow-up time, as reflected by the higher 2-year survival probability estimated for tislelizumab plus chemotherapy over placebo plus chemotherapy.

The present study demonstrated that tislelizumab plus chemotherapy outperformed comparator combination therapies in similar studies (13.5 months versus 10.3-12.8 months, respectively), with the caveat of cross trial comparison.

The OS benefit in the present study was sustained in the tislelizumab plus chemotherapy arm and was accompanied by improved PFS2 in favor of the tislelizumab arm, demonstrating the advantages of this therapy over chemotherapy in the first-line setting and regardless of subsequent anticancer treatment.

The patient population enrolled in the present study was broadly representative of patients with untreated ES-SCLC in China. SCLC primarily occurs in smokers, and the proportion of cases occurring in never-smokers has been reported to be only 2-5%. In the present study, the proportion of patients who were never-smokers (25%) was higher than global estimates (2.5%), consistent with the observed higher prevalence of never-smoking SCLC patients in Asia, as well as with prior phase 3 trials conducted in China in the same disease setting. The high incidence of never-smokers could be attributed to regional differences, while environmental (passive) tobacco smoking has been found to contribute to the development of SCLC in never-smokers. Although a consistent clinical benefit for OS and PFS was seen with tislelizumab plus chemotherapy versus placebo plus chemotherapy regardless of smoking status, no genomic information is yet available for the never-smoking SCLC patients of this study due to limited sample collection.

Carboplatin or cisplatin are recommended as the platinum component of first-line ES-SCLC regimens, in combination with etoposide. However, most ES-SCLC phase 3 trials demonstrating an OS benefit with the addition of anti-PD-1 or anti-PD-L1 therapies to chemotherapy have been restricted to carboplatin-based regimens, with the exception of the CASPIAN study of the anti-PD-L1 therapy, durvalumab, which allowed investigators to choose carboplatin or cisplatin. The previous study failed to demonstrate a significant OS benefit with pembrolizumab plus chemotherapy as a first-line treatment for ES-SCLC (median OS 10.8 months in the pembrolizumab arm versus 9.7 months in the placebo arm; HR: 0.80; 95% CI 0.64-0.98). Therefore, the present study of tislelizumab plus chemotherapy is the first phase 3 clinical trial demonstrating an OS benefit with an anti-PD-1 therapy combined with either cisplatin or carboplatin plus etoposide in this setting, providing new evidence that could broaden treatment options for patients with ES-SCLC.

Tislelizumab plus chemotherapy is well-tolerated with a manageable safety profile, as approximately one-fifth of the patients received tislelizumab for more than one year, while few patients discontinued study treatment due to TRAEs.

In conclusion, the addition of tislelizumab to standard first-line chemotherapy with platinum and etoposide demonstrated a significant reduction in the risk of death for patients with ES-SCLC, which was further supported by improvements in PFS, ORR and DoR, and a manageable safety profile.

Overall, Tislelizumab in combination with chemotherapy as first-line treatment for patients with untreated ES-SCLC demonstrated significant clinical benefit and a manageable safety profile compared with placebo plus chemotherapy.

Incorporation by Reference: Various references such as patents, patent applications, and publications are cited herein, the disclosures of which are hereby incorporated by reference herein in their entireties.

1. A method of treating extensive stage small cell lung cancer (ES-SCLC) in a human subject in need thereof, comprising administering to the human subject a therapeutically effective amount of an anti-PD-1 antibody or an antigen-binding fragment thereof, and a therapeutically effective amount of one or more chemotherapeutic drugs,
wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36.

2. The method of claim 1, wherein the ES-SCLC is:
(i) unresectable, locally advanced, and/or metastatic; or
(ii) stage IIIA, stage IIIB or stage IV.

3. (canceled)

4. (canceled)

5. (canceled)

6. (canceled)

7. The method of claim 1, wherein the subject has:
(i) liver, lung, lymph node, bone and/or adrenal gland metastasis, and/or (ii) does not have brain metastasis.

8. (canceled)

9. (canceled)

10. The method of claim 1, wherein the subject has:
(i) not been previously treated for the lung cancer or the treatment is a first line treatment, or
(ii) been previously treated with chemotherapy and/or immunotherapy.

11. (canceled)

12. The method of claim 1, wherein the heavy chain variable region of the anti-PD-1 antibody comprises the amino acid sequence of SEQ ID NO:24, and the light chain variable region of the anti-PD-1 antibody comprises the amino acid sequence of SEQ ID NO:26.

13. The method of claim 1, wherein the anti-PD-1 antibody comprises an IgG constant region comprising an amino acid sequence of SEQ ID NO:88.

14. The method of claim 1, wherein the anti-PD-1 antibody is administered:
(i) intravenously in the amount of 200 mg,
(ii) once every three weeks, and/or
(iii) by IV infusion.

15. The method of claim 1, wherein the one or more chemotherapeutic drug is a platinum chemotherapy drug.

16. The method of claim 1, wherein the one or more chemotherapeutic drug is carboplatin or cisplatin.

17. The method of claim 16, wherein the cisplatin is administered intravenously at about 75 mg/m² or the carboplatin is administered:
(i) intravenously at about area under the plasma or serum concentration-time curve 5 (AUC5),
(ii) once every three weeks (Q3W), and/or
(iii) the administration is by IV infusion.

18. The method of claim 1, wherein the one or more chemotherapeutic drugs comprise etoposide.

19. The method of claim 18, wherein the etoposide is administered:
(i) intravenously at about 100 mg/m²,
(ii) three times every three weeks (Q3W), and/or
(iii) by IV infusion.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 1, wherein the ES-SCLC is small cell carcinoma, combined small cell carcinoma, neuroendocrine (NE)-high subtype, or NE-low subtype.

32. (canceled)

33. (canceled)

34. (canceled)

35. (canceled)

36. (canceled)

37. A method of treating an extensive stage small cell lung cancer (ES-SCLC) in a human subject in need thereof, comprising administering to the human subject a therapeutically effective amount of an anti-PD-1 antibody or an antigen-binding fragment thereof, and a therapeutically effective amount of one or more chemotherapeutic drugs;
wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36;
wherein the one or more chemotherapeutic drugs are (i) cisplatin or carboplatin, and (ii) etoposide;
wherein the subject has a metastatic or stage IV ES-SCLC; and
wherein the treating is clinically effective or the subject is a responder to the treating by at least one measure of response to treatment.

38. (canceled)

39. (canceled)

40. (canceled)

41. (canceled)

42. (canceled)

43. The method of claim 37, wherein the anti-PD-1 antibody is:
(i) administered intravenously in the amount of 200 mg, wherein the administering is once every three weeks by IV infusion;
(ii) Tislelizumab;
(iii) formulated for intravenous injection in a pharmaceutical composition comprising a histidine buffer, a surfactant, and a stabilizer,
(iv) administered at a dose from about 2 mg/kg to about 5 mg/kg,
(v) administered at 100 mg, 150 mg, 200, mg, 250 mg, 300 mg, 350 mg, 400 mg, 450 mg, 500 mg, 550 mg, 600 mg, 650 mg, 700 mg, 750 mg, or 800 mg,
(v) administered every week, every 2 weeks, every 3 weeks, every 4 weeks, every 5 weeks, or every 6 weeks, and/or
(vi) administered at 150 mg every two weeks, 200 mg tislelizumab every 3 weeks, 300 mg every four weeks, or 400 mg every six weeks.

44. (canceled)

45. (canceled)

46. (canceled)

47. (canceled)

48. The method of claim 37, wherein the response to treatment is measured by tumor volume, size or diameter, and wherein the treating is determined to be clinically effective if:
(i) the treating reduces tumor volume, size or diameter by at least 5%, at least 10%, at least 15%, at least 20%, at least 25%, at least 30%, at least 35%, at least 40%, at least 45%, at least 50%, or at least 75%,
(ii) the subject has greater than 30% probability of survival at 24 months from the treatment initiation,
(iii) the subject has greater than 15% or 20% probability of event-free survival at 12 months from the treatment initiation,
(iv) overall survival rate of a patient population at 24 months from the treatment initiation is greater than 30%, and/or (v) event-free survival rate of a patient population at 12 months from the treatment initiation is greater than 20%.

49. (canceled)

50. (canceled)

51. (canceled)

52. A method of treating lung cancer in a human subject in need thereof, comprising administering to the human subject:
(a) a therapeutically effective amount of an anti-PD-1 antibody or an antigen-binding fragment thereof, wherein the anti-PD-1 antibody comprises (i) a heavy chain variable region comprising complementarity determining region (CDR)-H1 comprising SEQ ID NO: 31, CDR-H2 comprising SEQ ID NO: 32, and CDR-H3 comprising SEQ ID NO: 33, and (ii) a light chain variable region comprising CDR-L1 comprising SEQ ID NO: 34, CDR-L2 comprising SEQ ID NO: 35, and CDR-L3 comprising SEQ ID NO: 36; and
(b) a therapeutically effective amount of a chemotherapeutic drug comprising a) a platinum compound, and b) paclitaxe or nab-paclitaxel.

53. The method of claim 52, wherein the anti-PD-1 antibody is:
(i) Tislelizumab,
(ii) formulated for intravenous injection in a pharmaceutical composition comprising a histidine buffer, a surfactant, and a stabilizer,
(iii) administered at a dose from about 2 mg/kg to about 5 mg/kg,
(iv) administered at 100 mg, 150 mg, 200, mg, 250 mg, 300 mg, 350 mg, 400 mg, 450 mg, 500 mg, 550 mg, 600 mg, 650 mg, 700 mg, 750 mg, or 800 mg,
(v) administered every week, every 2 weeks, every 3 weeks, every 4 weeks, every 5 weeks, or every 6 weeks, and/or
(vi) administered at 150 mg every two weeks, 200 mg tislelizumab every 3 weeks, 300 mg every four weeks, or 400 mg every six weeks.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. The method of claim 1, wherein the anti-PD-1 antibody is formulated in a pharmaceutical composition comprising a histidine buffer, a surfactant, and a stabilizer.

68. The method of claim 67, wherein the surfactant is a polysorbate, and wherein the stabilizer is trehalose.

69. (canceled)


SEQUENCE LISTING (107 sequences)

SEQ ID NO: 1-3 - skipped (3 entries)

SEQ ID NO: 4 - protein, 118 aa, Mus musculus
QVQLKESGPGLVAPSKNLSITCTVSGFSLTSYGVHWIRQPPGKGLEWLGVIWAGGSTNYNSALMSRLSISKDNSRSQVFLRMNSLQTDDTAMYYCARAYGNYWYIDVWGAGTTVTVSS

SEQ ID NO: 5 - skipped

SEQ ID NO: 6 - protein, 107 aa, Mus musculus
DIVMTQTPKFLLVSAGDRVTITCKASQSVSNDVAWYQQKPGQSPKLLINYAFHRFTGVPDRFTGSGYGTDFIFTISTVQAEDLAVYFCHQAYSSPYTFGGGTKLEMK

SEQ ID NO: 7 - skipped

SEQ ID NO: 8 - protein, 114 aa, Mus musculus
QIQLVQSGPELKKPGETVKISCKASGYTFTNYGMNWVKQAPGKGLKWMGWINNNNGEPTYAEEFKGRFAFSLETSASTAYLQINNLKNEDTATYFCARDVMDYWGQGTSVTVSS

SEQ ID NO: 9 - skipped

SEQ ID NO: 10 - protein, 110 aa, Mus musculus
DIVLTQSPASLAVSLGQRATISCRASESVDNYGYSFMHWYQQKPGQPPQLLIYRASNLESGIPARFSGSGSRTGFTLTINPVEADDVATYYCQQSKEYPTFGGGTKLEVK

SEQ ID NO: 11 - protein, 10 aa, Mus musculus
GFSLTSYGVH

SEQ ID NO: 12 - protein, 16 aa, Mus musculus
VIWAGGSTNYNSALMS

SEQ ID NO: 13 - protein, 12 aa, Mus musculus
ARAYGNYWYIDV

SEQ ID NO: 14 - protein, 11 aa, Mus musculus
KASQSVSNDVA

SEQ ID NO: 15 - protein, 7 aa, Mus musculus
YAFHRFT

SEQ ID NO: 16 - protein, 9 aa, Mus musculus
HQAYSSPYT

SEQ ID NO: 17 - protein, 10 aa, Mus musculus
GYTFTNYGMN

SEQ ID NO: 18 - protein, 17 aa, Mus musculus
WINNNNGEPTYAEEFKG

SEQ ID NO: 19 - protein, 7 aa, Mus musculus
ARDVMDY

SEQ ID NO: 20 - protein, 15 aa, Mus musculus
RASESVDNYGYSFMH

SEQ ID NO: 21 - protein, 7 aa, Mus musculus
RASNLES

SEQ ID NO: 22 - protein, 8 aa, Mus musculus
QQSKEYPT

SEQ ID NO: 23 - DNA, 354 bp, synthetic construct
  misc_feature 1..354: note = 317-4B6 cDNA-Vh
  source 1..354: mol_type = other DNA
caggtgcagctgcaggagtcgggaccaggactggtgaagccttcggagaccctgtccctcacctgcactgtctctgggttttcattaaccagctatggtgtacactggatccggcagcccccagggaagggactggagtggatcggggtcatatacgccgatggaagcacaaattataatccctccctcaagagtcgagtgaccatatcaaaagacacctccaagaaccaggtttccctgaagctgagctctgtgaccgctgcggacacggccgtgtattactgtgcgagagcctatggtaactactggtacatcgatgtctggggccaagggaccacggtcaccgtctcctca

SEQ ID NO: 24 - protein, 118 aa, synthetic construct
  REGION 1..118: note = 317-4B6 pro-Vh
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 25 - DNA, 321 bp, synthetic construct
  misc_feature 1..321: note = 317-4B6 cDNA-Vk
  source 1..321: mol_type = other DNA
gacatcgtgatgacccagtctccagactccctggctgtgtctctgggcgagagggccaccatcaactgcaagtccagcgagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggccaggggaccaagctggagatcaaa

SEQ ID NO: 26 - protein, 107 aa, synthetic construct
  REGION 1..107: note = 317-4B6 pro-Vk
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 27 - DNA, 342 bp, synthetic construct
  misc_feature 1..342: note = 326-4A3 cDNA-Vh
  source 1..342: mol_type = other DNA
caggtgcagctggtgcagagcggcagcgagctgaagaagcccggcgccagcgtgaaggtgagctgcaaggccagcggctacaccttcaccaactacggcatgaactgggtgagacaggcccccggccagggcctgaagtggatgggctggatcaacaacaacaacgccgagcccacctacgcccaggacttcagaggcagattcgtgttcagcctggacaccagcgccagcaccgcctacctgcagatcagcagcctgaagaccgaggacaccgccgtgtactactgcgccagagacgtgatggactactggggccagggcaccctggtgaccgtgagcagc

SEQ ID NO: 28 - protein, 114 aa, synthetic construct
  REGION 1..114: note = 326-4A3 pro-Vh
QVQLVQSGSELKKPGASVKVSCKASGYTFTNYGMNWVRQAPGQGLKWMGWINNNNAEPTYAQDFRGRFVFSLDTSASTAYLQISSLKTEDTAVYYCARDVMDYWGQGTLVTVSS

SEQ ID NO: 29 - DNA, 330 bp, synthetic construct
  misc_feature 1..330: note = 326-4A3 cDNA-Vk
  source 1..330: mol_type = other DNA
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctgggaccgatttcaccctcacaattaatcctgtggaagctgaggatactgcaaattattactgtcagcaaagtaaagaatatccgacgttcggcggagggaccaaggtggagatcaaa

SEQ ID NO: 30 - protein, 110 aa, synthetic construct
  REGION 1..110: note = 326-4A3 pro-Vk
DIVLTQSPASLAVSPGQRATITCRASESVDNYGYSFMHWYQQKPGQPPKLLIYRASNLESGVPARFSGSGSGTDFTLTINPVEAEDTANYYCQQSKEYPTFGGGTKVEIK

SEQ ID NO: 31 - protein, 10 aa, Mus musculus
GFSLTSYGVH

SEQ ID NO: 32 - protein, 16 aa, synthetic construct
  REGION 1..16: note = 317-4B6 H-CDR2 or CDR-H2
VIYADGSTNYNPSLKS

SEQ ID NO: 33 - protein, 12 aa, Mus musculus
ARAYGNYWYIDV

SEQ ID NO: 34 - protein, 11 aa, synthetic construct
  REGION 1..11: note = 317-4B6 L-CDR1 or CDR-L1
KSSESVSNDVA

SEQ ID NO: 35 - protein, 7 aa, Mus musculus
YAFHRFT

SEQ ID NO: 36 - protein, 9 aa, Mus musculus
HQAYSSPYT

SEQ ID NO: 37 - protein, 10 aa, Mus musculus
GYTFTNYGMN

SEQ ID NO: 38 - protein, 17 aa, synthetic construct
  REGION 1..17: note = 326-4A3 H-CDR2 or CDR-H2
WINNNNAEPTYAQDFRG

SEQ ID NO: 39 - protein, 7 aa, Mus musculus
ARDVMDY

SEQ ID NO: 40 - protein, 15 aa, Mus musculus
RASESVDNYGYSFMH

SEQ ID NO: 41 - protein, 7 aa, Mus musculus
RASNLES

SEQ ID NO: 42 - protein, 8 aa, Mus musculus
QQSKEYPT

SEQ ID NO: 43 - protein, 118 aa, synthetic construct
  REGION 1..118: note = 317-4B2 pro-Vh
QVQLQESGPGLVKPSETLSLTCTVSGFSLTSYGVHWIRQPPGKGLEWIGVIYAGGSTNYNPSLKSRVTISKDTSKNQFSLKLSSVTAADTAVYYCARAYGNYWYIDVWGQGTTVTVSS

SEQ ID NO: 44 - protein, 107 aa, synthetic construct; identical to SEQ ID NO: 26
  REGION 1..107: note = 317-4B2 pro-Vk

SEQ ID NO: 45 - protein, 118 aa, synthetic construct
  REGION 1..118: note = 317-4B5 pro-Vh
QVQLQESGPGLVKPSETLSLTCTVSGFSLTSYGVHWIRQPPGKGLEWIGVIYAGGSTNYNPSLKSRVTISKDTSKNQVSLKLSSVTAADTAVYYCARAYGNYWYIDVWGQGTTVTVSS

SEQ ID NO: 46 - protein, 107 aa, synthetic construct; identical to SEQ ID NO: 26
  REGION 1..107: note = 317-4B5 pro-Vk

SEQ ID NO: 47 - DNA, 354 bp, synthetic construct
  misc_feature 1..354: note = 317-1 cDNA-Vh
  source 1..354: mol_type = other DNA
caggtgcagctgcaggagtcgggaccaggactggtgaagccttcggagaccctgtccctcacctgcactgtctctgggttttcattaaccagctatggtgtacactggatccggcagcccccagggaagggactggagtggctgggggtcatatgggccggtggaagcacaaattataatccctccctcaagagtcgactgaccatatcaaaagacaactccaagagccaggtttccctgaagatgagctctgtgaccgctgcggacacggccgtgtattactgtgcgagagcctatggtaactactggtacatcgatgtctggggccaagggaccacggtcaccgtctcctca

SEQ ID NO: 48 - protein, 118 aa, synthetic construct
  REGION 1..118: note = 317-1 pro-Vh
QVQLQESGPGLVKPSETLSLTCTVSGFSLTSYGVHWIRQPPGKGLEWLGVIWAGGSTNYNPSLKSRLTISKDNSKSQVSLKMSSVTAADTAVYYCARAYGNYWYIDVWGQGTTVTVSS

SEQ ID NO: 49 - DNA, 321 bp, synthetic construct
  misc_feature 1..321: note = 317-1 cDNA-Vk
  source 1..321: mol_type = other DNA
gacatcgtgatgacccagtctccagactccctggctgtgtctctgggcgagagggccaccatcaactgcaaggccagccagagtgtgagtaatgatgtagcttggtaccagcagaaaccaggacagcctcctaagctgctcattaactatgcatttcatcgcttcactggggtccctgaccgattcagtggcagcgggtatgggacagatttcactctcaccatcagcagcctgcaggctgaagatgtggcagtttattactgtcaccaggcttatagttctccgtacacgtttggcggggggaccaagctggagatcaaa

SEQ ID NO: 50 - protein, 107 aa, synthetic construct
  REGION 1..107: note = 317-1 pro-Vk
DIVMTQSPDSLAVSLGERATINCKASQSVSNDVAWYQQKPGQPPKLLINYAFHRFTGVPDRFSGSGYGTDFTLTISSLQAEDVAVYYCHQAYSSPYTFGGGTKLEIK

SEQ ID NO: 51 - protein, 114 aa, synthetic construct
  REGION 1..114: note = 326-3B1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 52 - protein, 110 aa, synthetic construct
  REGION 1..110: note = 326-3B1 pro-Vk
DIVLTQSPASLAVSPGQRATITCRASESVDNYGYSFMHWYQQKPGQPPKLLIYRASNLESGVPARFSGSGSRTDFTLTINPVEANDTANYYCQQSKEYPTFGGGTKVEIK

SEQ ID NO: 53 - protein, 114 aa, synthetic construct
  REGION 1..114: note = 326-3G1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTLVTVSS

SEQ ID NO: 54 - protein, 110 aa, synthetic construct; identical to SEQ ID NO: 52
  REGION 1..110: note = 326-3G1 pro-Vk

SEQ ID NO: 55 - DNA, 342 bp, synthetic construct
  misc_feature 1..342: note = 326-1 cDNA-Vh
  source 1..342: mol_type = other DNA
caggtgcagctggtgcagagcggcagcgagctgaagaagcccggcgccagcgtgaaggtgagctgcaaggccagcggctacaccttcaccaactacggcatgaactgggtgagacaggcccccggccagggcctggagtggatgggctggatcaacaacaacaacggcgagcccacctacgcccagggcttcagaggcagattcgtgttcagcctggacaccagcgccagcaccgcctacctgcagatcagcagcctgaagaccgaggacaccgccgtgtacttctgcgccagagacgtgatggactactggggccagggcaccaccgtgaccgtgagcagc

SEQ ID NO: 56 - protein, 114 aa, synthetic construct
  REGION 1..114: note = 326-1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYFCARDVMDYWGQGTTVTVSS

SEQ ID NO: 57 - DNA, 330 bp, synthetic construct
  misc_feature 1..330: note = 326-1 cDNA-Vk
  source 1..330: mol_type = other DNA
gacattgtgctgacccagtctccagcctccttggccgtgtctccaggacagagggccaccatcacctgcagagccagtgaaagtgttgataattatggctatagttttatgcactggtatcagcagaaaccaggacaacctcctaaactcctgatttaccgtgcatccaacctagaatctggggtcccagccaggttcagcggcagtgggtctaggaccgatttcaccctcacaattaatcctgtggaagctaatgatactgcaaattattactgtcagcaaagtaaagaatatccgacgttcggcggagggaccaaggtggagatcaaa

SEQ ID NO: 58 - protein, 110 aa, synthetic construct; identical to SEQ ID NO: 52
  REGION 1..110: note = 326-1 pro-Vk

SEQ ID NO: 59 - protein, 16 aa, synthetic construct
  REGION 1..16: note = 317-1 H-CDR2 or CDR-H2
VIWAGGSTNYNPSLKS

SEQ ID NO: 60 - protein, 16 aa, synthetic construct
  REGION 1..16: note = 317-4B2 H-CDR2 or CDR-H2
VIYAGGSTNYNPSLKS

SEQ ID NO: 61 - protein, 11 aa, synthetic construct
  REGION 1..11: note = 317-4B2 L-CDR1 or CDR-L1
KSSESVSNDVA

SEQ ID NO: 62 - protein, 17 aa, synthetic construct
  REGION 1..17: note = 326-1 H-CDR2 or CDR-H2
WINNNNGEPTYAQGFRG

SEQ ID NO: 63 - protein, 17 aa, synthetic construct
  REGION 1..17: note = 326-3G1 H-CDR2 or CDR-H2
WINNNNGEPTYAQDFRG

SEQ ID NO: 64 - protein, 118 aa, synthetic construct
  REGION 1..118: note = 317-3A1 pro-Vh
QVQLQESGPGLVKPSETLSLTCTVSGFSLTSYGVHWIRQPPGKGLEWIGVIWAGGSTNYNPSLKSRVTISVDTSKNQFSLKLSSVTAADTAVYYCARAYGNYWYIDVWGQGTTVTVSS

SEQ ID NO: 65 - protein, 118 aa, synthetic construct
  REGION 1..118: note = 317-3C1 pro-Vh
QVQLQESGPGLVKPSETLSLTCTVSGFSLTSYGVHWIRQPPGKGLEWLGVIWAGGSTNYNPSLKSRVTISVDTSKNQFSLKLSSVTAADTAVYYCARAYGNYWYIDVWGQGTTVTVSS

SEQ ID NO: 66 - protein, 118 aa, synthetic construct
  REGION 1..118: note = 317-3E1 pro-Vh
QVQLQESGPGLVKPSETLSLTCTVSGFSLTSYGVHWIRQPPGKGLEWIGVIWAGGSTNYNPSLKSRVTISVDTSKSQFSLKLSSVTAADTAVYYCARAYGNYWYIDVWGQGTTVTVSS

SEQ ID NO: 67 - protein, 118 aa, synthetic construct
  REGION 1..118: note = 317-3F1 pro-Vh
QVQLQESGPGLVKPSETLSLTCTVSGFSLTSYGVHWIRQPPGKGLEWIGVIWAGGSTNYNPSLKSRVTISVDTSKNQVSLKLSSVTAADTAVYYCARAYGNYWYIDVWGQGTTVTVSS

SEQ ID NO: 68 - protein, 118 aa, synthetic construct
  REGION 1..118: note = 317-3G1 pro-Vh
QVQLQESGPGLVKPSETLSLTCTVSGFSLTSYGVHWIRQPPGKGLEWIGVIWAGGSTNYNPSLKSRVTISVDTSKSQVSLKLSSVTAADTAVYYCARAYGNYWYIDVWGQGTTVTVSS

SEQ ID NO: 69 - protein, 118 aa, synthetic construct
  REGION 1..118: note = 317-3H1 pro-Vh
QVQLQESGPGLVKPSETLSLTCTVSGFSLTSYGVHWIRQPPGKGLEWIGVIWAGGSTNYNPSLKSRVTISKDTSKSQVSLKLSSVTAADTAVYYCARAYGNYWYIDVWGQGTTVTVSS

SEQ ID NO: 70 - protein, 118 aa, synthetic construct
  REGION 1..118: note = 317-3I1 pro-Vh
QVQLQESGPGLVKPSETLSLTCTVSGFSLTSYGVHWIRQPPGKGLEWIGVIWAGGSTNYNPSLKSRVTISKDTSKNQVSLKLSSVTAADTAVYYCARAYGNYWYIDVWGQGTTVTVSS

SEQ ID NO: 71 - protein, 118 aa, synthetic construct
  REGION 1..118: note = 317-4B1 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYWYIDVWGQGTTVTVSS

SEQ ID NO: 72 - protein, 118 aa, synthetic construct
  REGION 1..118: note = 317-4B3 pro-Vh
QVQLQESGPGLVKPSETLSLTCTVSGFSLTSYGVHWIRQPPGKGLEWIGVINAGGSTNYNPSLKSRVTISKDTSKNQFSLKLSSVTAADTAVYYCARAYGNYWYIDVWGQGTTVTVSS

SEQ ID NO: 73 - protein, 118 aa, synthetic construct
  REGION 1..118: note = 317-4B4 pro-Vh
QVQLQESGPGLVKPSETLSLTCTVSGFSLTSYGVHWIRQPPGKGLEWIGVIWAGGSTNYNPSLKSRVTISKDTSKNQFSLKLSSVTAADTAVYYCARAYGNYPYIDVWGQGTTVTVSS

SEQ ID NO: 74 - protein, 107 aa, synthetic construct
  REGION 1..107: note = 317-4A2 pro-Vk
DIVMTQSPDSLAVSLGERATINCKSSQSVSNDVAWYQQKPGQPPKLLINYAFHRFTGVPDRFSGSGYGTDFTLTISSLQAEDVAVYYCHQAYSSPYTFGQGTKLEIK

SEQ ID NO: 75 - protein, 114 aa, synthetic construct
  REGION 1..114: note = 326-3A1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 76 - protein, 114 aa, synthetic construct
  REGION 1..114: note = 326-3C1 pro-Vh
QVQLVQSGPELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 77 - protein, 114 aa, synthetic construct
  REGION 1..114: note = 326-3D1 pro-Vh
QVQLVQSGSELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTLVTVSS

SEQ ID NO: 78 - protein, 114 aa, synthetic construct
  REGION 1..114: note = 326-3E1 pro-Vh
QVQLVQSGSELKKPGASVKVSCKASGYTFTNYGMNWVRQAPGQGLKWMGWINNNNGEPTYAQDFRGRFVFSLDTSASTAYLQISSLKTEDTAVYYCARDVMDYWGQGTTVTVSS

SEQ ID NO: 79 - protein, 114 aa, synthetic construct
  REGION 1..114: note = 326-3F1 pro-Vh
QVQLVQSGPELKKPGASVKVSCKASGYTFTNYGMNWVRQAPGQGLEWMGWINNNNGEPTYAQGFRGRFVFSLDTSASTAYLQISSLKTEDTAVYYCARDVMDYWGQGTTVTVSS

SEQ ID NO: 80 - protein, 114 aa, synthetic construct
  REGION 1..114: note = 326-3B N55D pro-Vh
QVQLVQSGSELKKPGASVKVSCKASGYTFTNYGMNWVRQAPGQGLKWMGWINNNDGEPTYAQDFRGRFVFSLDTSASTAYLQISSLKTEDTAVYYCARDVMDYWGQGTLVTVSS

SEQ ID NO: 81 - protein, 110 aa, synthetic construct
  REGION 1..110: note = 326-4A1 pro-Vk
DIVLTQSPASLAVSPGQRATITCRASESVDNYGYSFMHWYQQKPGQPPKLLIYRASNLESGVPARFSGSGSRTDFTLTINPVEAEDTANYYCQQSKEYPTFGGGTKVEIK

SEQ ID NO: 82 - protein, 110 aa, synthetic construct
  REGION 1..110: note = 326-4A2 pro-Vk
DIVLTQSPASLAVSPGQRATITCRASESVDNYGYSFMHWYQQKPGQPPKLLIYRASNLESGVPARFSGSGSGTDFTLTINPVEANDTANYYCQQSKEYPTFGGGTKVEIK

SEQ ID NO: 83 - protein, 327 aa, synthetic construct
  REGION 1..327: note = huIgG4mt1 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 327 aa, synthetic construct
  REGION 1..327: note = huIgG4mt2 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 327 aa, synthetic construct
  REGION 1..327: note = huIgG4mt6 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 327 aa, synthetic construct
  REGION 1..327: note = huIgG4mt8 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVTVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 327 aa, synthetic construct
  REGION 1..327: note = huIgG4mt9 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 88 - protein, 327 aa, synthetic construct
  REGION 1..327: note = huIgG4mt10 pro
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 89 - protein, 367 aa, synthetic construct
  REGION 1..367: note = OS8 pro
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINSSVVPVLQKVNSTTTKPVLRTPSPVHPTGTSQPQRPEDCRPRGSVKGTGLDFACDIYIWAPLAGICVALLLSLIITLICYHRSRKRVCKCPRPLVRQEGKPRPSEKIV

SEQ ID NO: 90 - skipped

SEQ ID NO: 91 - protein, 327 aa, synthetic construct
  REGION 1..327: note = IgG4 Fc region 228P-233P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPEFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 327 aa, synthetic construct
  REGION 1..327: note = IgG4 Fc region 228P-234V
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 327 aa, synthetic construct
  REGION 1..327: note = IgG4 Fc region 228P-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 327 aa, synthetic construct
  REGION 1..327: note = IgG4 Fc region 228P-234V-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 327 aa, synthetic construct
  REGION 1..327: note = IgG4 Fc region 228P-234A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAFGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 327 aa, synthetic construct
  REGION 1..327: note = IgG4 Fc region 228P-234A-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 327 aa, synthetic construct
  REGION 1..327: note = IgG4 Fc region 228P-233P-234A-235A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 327 aa, synthetic construct
  REGION 1..327: note = IgG4 Fc region 228P-234V-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEVAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 327 aa, synthetic construct
  REGION 1..327: note = IgG4 Fc region 228P-234A-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 327 aa, synthetic construct
  REGION 1..327: note = IgG4 Fc region 228P-233P-234A-235A-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 327 aa, synthetic construct
  REGION 1..327: note = IgG4 Fc region 228P-265A
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 327 aa, synthetic construct
  REGION 1..327: note = IgG4 Fc region 228P-309V
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 327 aa, synthetic construct
  REGION 1..327: note = IgG4 Fc region 228P-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 104 - protein, 327 aa, synthetic construct
  REGION 1..327: note = IgG4 Fc region 228P-309V-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 105 - protein, 327 aa, synthetic construct
  REGION 1..327: note = IgG4 Fc region 228P-265A-309V-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 106 - protein, 327 aa, synthetic construct
  REGION 1..327: note = IgG4 Fc region 228P-233P-234A-235A-265A-309V-409K
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPAAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 107 - skipped